SEQUENCE LISTING (120 sequences)

SEQ ID NO: 1 - protein, 122 aa, Artificial Sequence
  source: /note=“Description of Artificial Sequence Synthetic polypeptide”
QVQLQQSGPELVKPGASVKISCKASGYAFSSSWMNWVKQRPGKGLEWIGRTYPGDGDTNYNGKFKGKATLTADKSSSTAYMQLSSLTSEDSAVYFCARGWILADGGYSFAYWGQGTLVTVSA

SEQ ID NO: 2 - protein, 112 aa, Artificial Sequence
  source: /note=“Description of Artificial Sequence Synthetic polypeptide”
DIVMTQAAPSIPVTPGESVSISCRSSKSLLHSNGNTYLYWFLQRPGQSPQLLIYRMSNLASGVPDRFSGSGSGTAFTLRISRVEAEDVGIYYCMQHLEYPFTFGTGTKLEIK

SEQ ID NO: 3 - protein, 118 aa, Artificial Sequence
  source: /note=“Description of Artificial Sequence Synthetic polypeptide”
QVQLQQSGPELVKPGASVKISCKASGYAFSSSWMNWVKQRPGKGLEWIGRIYPGDGDTNYNGKFKGKATLTADKSSSTAYIQLSSLTSEDSAVYFCARGYADYSFAYWGQGTLVTVSA

SEQ ID NO: 4 - protein, 112 aa, Artificial Sequence
  source: /note=“Description of Artificial Sequence Synthetic polypeptide”
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGSGTKLEIK

SEQ ID NO: 5 - protein, 118 aa, Artificial Sequence
  source: /note=“Description of Artificial Sequence Synthetic polypeptide”
QVQLQQSGPELVKPGASVKISCKASGYAFSSSWMNWVKQRPGKGLEWIGRIYPGDGETNYNGKFKGKATLTADKSSNTAYMQLSSLTSEDSAVYFCARGFGDYSFAYWGQGTLVTVSA

SEQ ID NO: 6 - protein, 112 aa, Artificial Sequence
  source: /note=“Description of Artificial Sequence Synthetic polypeptide”
DIVMTQAAPSVPVTPGESVSISCRSSKSLLHSNGNTYLYWFLQRPGQSPQLLIYRMSNLASGAPDRFSGSGSGTAFTLRISRVETEDVGVYYCMQHLEYPYTFGSGTKLEIK

SEQ ID NO: 7 - protein, 118 aa, Artificial Sequence
  source: /note=“Description of Artificial Sequence Synthetic polypeptide”
QVQLQQSGPELVKPGASVKISCKASGYTFSSSWMNWVKQRPGKGLEWIGRIYPGDGDTNYNGKFKGKATLTADKSSSTAYMQLSSLTSEDSAVYFCASGYADYSFAYWGQGTLVTVSA

SEQ ID NO: 8 - protein, 112 aa, Artificial Sequence; identical to SEQ ID NO: 4
  source: /note=“Description of Artificial Sequence Synthetic polypeptide”

SEQ ID NO: 9 - protein, 118 aa, Artificial Sequence
  source: /note=“Description of Artificial Sequence Synthetic polypeptide”
QVQLQQSGPELVKPGASVKISCKASGYAFSRSWMNWVKQRPGKGLEWIGRIYPGDGDTNYNGKFKGKATLTADKSSSTAYMQLSSLTSEDSAVYFCASGYDDYSFAYWGQGTLVTVSA

SEQ ID NO: 10 - protein, 112 aa, Artificial Sequence; identical to SEQ ID NO: 4
  source: /note=“Description of Artificial Sequence Synthetic polypeptide”

SEQ ID NO: 11 - protein, 118 aa, Artificial Sequence
  source: /note=“Description of Artificial Sequence Synthetic polypeptide”
QVQLQQSGPELVKPGASVKISCRAFGYAFSNSWMNWVKQRPGKGLEWIGRIYPGDGETNNNGKFKGKATLTADKSSSTAYMQLSSLTSEDSAVYFCARGYGDYSFAYWGQGTLVTVSA

SEQ ID NO: 12 - protein, 112 aa, Artificial Sequence
  source: /note=“Description of Artificial Sequence Synthetic polypeptide”
DIVMTQAAPSVPVTPGESVSISCRSSKSLLHSNGNTYLYWFLQRPGQSPQLLIYRMSNLASGVPDRFSGSGSGAAFTLRISRVEAEDVGVYYCMQHLEYPYTFGSGTKLEIK

SEQ ID NO: 13 - protein, 115 aa, Artificial Sequence
  source: /note=“Description of Artificial Sequence Synthetic polypeptide”
QVQLQQPGAELVKPGASVKLSCKASGYTFTNYWVNWVKQRPGRGLEWIGRIHPSDSETHCNQKFKRKATLTVNKSSSTAYIQLHSLTSEDSAVYYCTSGGWFAYWGQGTLVTVSA

SEQ ID NO: 14 - protein, 112 aa, Artificial Sequence
  source: /note=“Description of Artificial Sequence Synthetic polypeptide”
DIVMTQAAPSVPVTPGESVSISCRSSKSLLYSNGNIYLYWFLQRPGQSPQLLIYRMSNLASGVPDRFSGSGSGTAFTLRISRVEAEDVGVYYCMQHLEYPYTFGSGTKLEIK

SEQ ID NO: 15 - protein, 118 aa, Artificial Sequence
  source: /note=“Description of Artificial Sequence Synthetic polypeptide”
QVQLQQSGPELVKPGASVKISCKASGYAFSSSWMNWVKQRPGKGLEWIGRIYPGDGETNNNGKFKGKATLTADKSSTTAYMQLSSLTSEDSAVYFCARGYGDYSFAYWGQGTLVTVSA

SEQ ID NO: 16 - protein, 112 aa, Artificial Sequence; identical to SEQ ID NO: 12
  source: /note=“Description of Artificial Sequence Synthetic polypeptide”

SEQ ID NO: 17 - protein, 118 aa, Artificial Sequence
  source: /note=“Description of Artificial Sequence Synthetic polypeptide”
QVQLQQSGPELVKPGASVKISCKASGYAFSTSWMNWVKQRPGKGLEWIGRIYPGDGEANYNGKFKGKATLTADKSSSSAYMQLSSLTSEDSAVYFCARGYGDYSFAYWGQGTLVTVSA

SEQ ID NO: 18 - protein, 112 aa, Artificial Sequence
  source: /note=“Description of Artificial Sequence Synthetic polypeptide”
DIVMTQAAPSVPVTPGESVSISCRSSKSLLHSNGNTYLYWFMQRPGQSPQLLIYRMSNLASGVPDRFSGSGSGTAFTLRISRVEAEDVGVYYCMQHVEYPYTFGSGTKLEIK

SEQ ID NO: 19 - protein, 118 aa, Artificial Sequence
  source: /note=“Description of Artificial Sequence Synthetic polypeptide”
QVQLQQSGPELVKPGASVKISCKASGYAFSSSWMNWVKQRPGKGPEWIGRIYPGDGETNYNGKFKGKATLTADKSSSTVYMQLSSLTSEDSAVYFCARGYGDYSFAYWGQGTLVTVSA

SEQ ID NO: 20 - protein, 112 aa, Artificial Sequence; identical to SEQ ID NO: 4
  source: /note=“Description of Artificial Sequence Synthetic polypeptide”

SEQ ID NO: 21 - protein, 118 aa, Artificial Sequence
  source: /note=“Description of Artificial Sequence Synthetic polypeptide”
QVQLQQSGPELLNPGASVKISCKASGYAFSRSWMNWVKQRPGKGLEWIGRIYPGDGETNYNGKFKGKATLTADKSSTTAYMQFSSLTSEDSAVYFCARGDGDYSFAYWGQGTLVTVSA

SEQ ID NO: 22 - protein, 112 aa, Artificial Sequence; identical to SEQ ID NO: 4
  source: /note=“Description of Artificial Sequence Synthetic polypeptide”

SEQ ID NO: 23 - protein, 118 aa, Artificial Sequence
  source: /note=“Description of Artificial Sequence Synthetic polypeptide”
QVQLQQSGPELVKPGASVKISCKASGYAFTNSWMNWVKQRPGKGLEWIGRIYPGDGETIYNGKFRVKATLTADKSSSTAYMDISSLTSEDSAVYFCARGYDDYSFAYWGQGTLVTVSA

SEQ ID NO: 24 - protein, 112 aa, Artificial Sequence
  source: /note=“Description of Artificial Sequence Synthetic polypeptide”
DIVMTQAAPSIPVTPGESVSISCRSSKSLLHSNGNTYLYWFLQRPGQSPQLLIYRMSNLASGVPDRFSGSGSGTAFTLRISRVEAEDVGVYYCMQHIEYPFTFGSGTKLEIK

SEQ ID NO: 25 - protein, 115 aa, Artificial Sequence
  source: /note=“Description of Artificial Sequence Synthetic polypeptide”
QVQLQQPGTELVRPGASVKLSCKASGYTFTDYWVNWVKQRPGRGLEWIGRIHPYDSETHYNQKFKNKATLTVDKSSSTAYIQLSSLTSEDSAVYYCASGGWFASWGQGTLVTVSA

SEQ ID NO: 26 - protein, 112 aa, Artificial Sequence
  source: /note=“Description of Artificial Sequence Synthetic polypeptide”
DIVMTQAAPSVPVTPGESVSISCRSSKSLLYSNGNTYLYWFLQRPGQSPQLLIYRMSNLASGVPDRFSGSGSGTAFTLTISSVEAEDVGVYYCMQHLEYPYTFGSGTKLEIK

SEQ ID NO: 27 - protein, 115 aa, Artificial Sequence
  source: /note=“Description of Artificial Sequence Synthetic polypeptide”
QVQLQQPGAELVKPGASVKLSCKASGYTFTDYWMNWVKQRPGRGLEWIGRIHPFDSETHCSQKFKNKATLTVDKSSNTAYIQFSSLTSEDSAVYYCSSGGWFAYWGQGTLVTVSA

SEQ ID NO: 28 - protein, 112 aa, Artificial Sequence
  source: /note=“Description of Artificial Sequence Synthetic polypeptide”
DIVMTQAAPSVSVTPGESVSISCRSSKSLLYSNGNIYLYWFLQRPGQSPQLLIYRMSNLASGVPDRFSGSGSGTAFTLKISRVEAEDVGVYYCMQHLEYPYTFGSGTKLEIK

SEQ ID NO: 29 - protein, 118 aa, Artificial Sequence
  source: /note=“Description of Artificial Sequence Synthetic polypeptide”
QVQLQQSGPELVKPGASVKISCKASGYAFTNSWMNWVRQRPGKGLEWIGRIYPGDGETIYNGKFRVKATLTADKSSSTAYMEISSLTSEDSAVYFCARGYDDYSFAYWGQGTLVTVSA

SEQ ID NO: 30 - protein, 112 aa, Artificial Sequence
  source: /note=“Description of Artificial Sequence Synthetic polypeptide”
DIVMTQAAPSVPVTPGESVSISCRSSKSLLHNNGNTYLYWFLQRPGQSPQLLIYRMSNLASGVPDRFSGSGSGTAFTLRISRVEAEDVGVYYCMQHIEYPFTFGSGTKLEIK

SEQ ID NO: 31 - protein, 118 aa, Artificial Sequence
  source: /note=“Description of Artificial Sequence Synthetic polypeptide”
QVQLQQSGPELVKPGASVKISCKASGYAFSNSWMNWVNQRPGKGLEWIGRIYPGDGDTIYNGNFKGKATLTADKSSSIAYMQLSSLTSEDSAVYFCTSGYDDYSFAYWGQGTLVTVSA

SEQ ID NO: 32 - protein, 112 aa, Artificial Sequence
  source: /note=“Description of Artificial Sequence Synthetic polypeptide”
DIVMTQAAPSLPVTPGESVSISCRSSKSLLHSNGNTYLYWFLQRPGQSPQLLIYRMSNLASGVPDRFSGSGSGTAFTLRISRVEAEDVGVYYCMQHLEYPYTFGSGTKLEIK

SEQ ID NO: 33 - protein, 141 aa, Artificial Sequence
  source: /note=“Description of Artificial Sequence Synthetic polypeptide”
MVLASSTTSIHTMLLLLLMLAQPAMAEVKLVESGGGLVKPGGSRKLSCAASGFTFSSYTMSWVRQTPAKRLEWVATISSGSSTIYYADTVKGRFTISRDNAKNTLFLQMTSLRSEDTAMYYCARRWFLDCWGQGTTLTVSS

SEQ ID NO: 34 - protein, 119 aa, Artificial Sequence
  source: /note=“Description of Artificial Sequence Synthetic polypeptide”
DIVLTQSPASLAVSLGQSVTISCRASESVEYYGTSLMQWYQQKPGQPPKLLIYGASNVESGVPARFSGSGSGTDFSLNIHPVEEDDIAMYFCQQSRKVPWTFGGGTKLEIKDYKDDDDK

SEQ ID NO: 35 - protein, 144 aa, Artificial Sequence
  source: /note=“Description of Artificial Sequence Synthetic polypeptide”
MVLASSTTSIHTMLLLLLMLAQPAMAQVQLQQSGPELVKPGASVKISCKASGYAFSSSWMNWMKQRPGKGLEWIGRIYPGDGDTNYNGKFKGKATLTADKSSSTAYMQLSSLTSEDSAVYFCARARKTSWFAYWGQGTLVTVSA

SEQ ID NO: 36 - protein, 115 aa, Artificial Sequence
  source: /note=“Description of Artificial Sequence Synthetic polypeptide”
DIVLTQSQKFMSTSVGDRVSISCKASQNVGNIIAWYQQKPGQSPKALIYLASYRYSGVPDRFTGSGSGTDFTLTISNVQSEDLAEYFCQQYSSSPLTFGAGTKLEIKDYKDDDDK

SEQ ID NO: 37 - protein, 116 aa, Artificial Sequence
  source: /note=“Description of Artificial Sequence Synthetic polypeptide”
DVQLQESGPGLVKPSQSLSLTCTVTGYSITSDYAWSWIRQLPGNKLEWMGYITYSGYSIYNPSLKSRISISRDTSKNQLFLQLNSVTTEDTATYYCVGGYDNMDYWGQGTSVTVSS

SEQ ID NO: 38 - protein, 108 aa, Artificial Sequence
  source: /note=“Description of Artificial Sequence Synthetic polypeptide”
QIVLTQSPAIMSASPGEKVTLTCSASSSVSSSHLYWYQQKPGSSPKLWIYSTSNLASGVPARFSGSGSGTSYSLTISNMETEDAASYFCHQWSSYPWTFGGGTKLEIK

SEQ ID NO: 39 - protein, 118 aa, Artificial Sequence
  source: /note=“Description of Artificial Sequence Synthetic polypeptide”
QVQLVQSGPEVKKPGASVKVSCKASGYTFTNSWMNWVRQRPGKGLEWMGRIYPGDGETIYNGKFRVRVTITADESTSTAYMELSSLRSEDTAVYYCARGYDDYSFAYWGQGTTVTVSS

SEQ ID NO: 40 - protein, 112 aa, Artificial Sequence
  source: /note=“Description of Artificial Sequence Synthetic polypeptide”
DIVMTQSALSLPVTPGEPASISCRSSKSLLHSNGNTYLYWFQQKPGQSPQLLIYRMSNLASGVPDRFSGSGSGTAFTLKISRVEAEDVGVYYCMQHIEYPFTFGQGTKLEIK

SEQ ID NO: 41 - protein, 118 aa, Artificial Sequence
  source: /note=“Description of Artificial Sequence Synthetic polypeptide”
QVQLVQSGPEVKKPGASVKVSCKASGYTFTNSWMNWVRQRPGKGLEWVGRIYPGDGETIYNGKFRVRVTITADESTSTAYMELSSLRSEDTAVYYCARGYDDYSFAYWGQGTTVTVSS

SEQ ID NO: 42 - protein, 112 aa, Artificial Sequence
  source: /note=“Description of Artificial Sequence Synthetic polypeptide”
DIVMTQSALSLPVTPGEPASISCRSSKSLLHSNGNTYLYWYLQKPGQSPQLLIYRMSNLASGVPDRFSGSGSGTAFTLKISRVEAEDVGVYYCMQHIEYPFTFGQGTKLEIK

SEQ ID NO: 43 - protein, 118 aa, Artificial Sequence
  source: /note=“Description of Artificial Sequence Synthetic polypeptide”
QVQLVQSGPEVKKPGASVKVSCKASGYTFTNSWMNWIRQRPGKGLEWIGRIYPGDGETIYNGKFRVRVTITADESTSTAYMELSSLRSEDTAVYYCARGYDDYSFAYWGQGTLVTVSS

SEQ ID NO: 44 - protein, 112 aa, Artificial Sequence; identical to SEQ ID NO: 42
  source: /note=“Description of Artificial Sequence Synthetic polypeptide”

SEQ ID NO: 45 - protein, 118 aa, Artificial Sequence
  source: /note=“Description of Artificial Sequence Synthetic polypeptide”
QVQLVQSGPEVKKPGASVKVSCKASGYTFTNSWMNWVRQRPGKGLEWIGRIYPGDGETIYNGKFRVRVTITADESTSTAYMQLSSLRSEDTAVYYCARGYDDYSFAYWGQGTTVTVSS

SEQ ID NO: 46 - protein, 112 aa, Artificial Sequence
  source: /note=“Description of Artificial Sequence Synthetic polypeptide”
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHIEYPFTFGQGTKLEIK

SEQ ID NO: 47 - protein, 118 aa, Artificial Sequence
  source: /note=“Description of Artificial Sequence Synthetic polypeptide”
QVQLVQSGPEVKKPGASVKVSCKASGYTFTNSWMNWVRQRPGKGLEWIGRIYPGDGETIYNGKFRVRVTITADESTSTAYMELSSLRSEDTAVYYCARGYDDYSFAYWGQGTTVTVSS

SEQ ID NO: 48 - protein, 112 aa, Artificial Sequence
  source: /note=“Description of Artificial Sequence Synthetic polypeptide”
DIVMTQSPLSLPVTPGEPASISCRSSKSLLHSNGNTYLYWFQQKPGQAPRLLIYRMSNLASGVPDRFSGSGSGTAFTLKISRVEAEDVGVYYCMQHIEYPFTFGQGTKLEIK

SEQ ID NO: 49 - protein, 249 aa, Artificial Sequence
  source: /note=“Description of Artificial Sequence Synthetic polypeptide”
MAQVQLQESGGEMKKPGESLKISCKGYGYSFATSWIGWVRQMPGRGLEWMAIMYPGNSDTRHNPSFEDQVTMSADTSINTAYLQWSSLKASDTAMYYCARAGVAGGAFDLWGKGTMVTVSSGGGGSGGGGSGGGGSQSVLTQPASVSGSPGQSITISCTGTSSGVGGYNYVSWYQQHPGKAPKLLIYGNSNRPSGVPDRFSASKSGNTASLTISGLQAEDEADYFCSTYAPPGIIMFGGGTKLTVLGAA

SEQ ID NO: 50 - protein, 245 aa, Artificial Sequence
  source: /note=“Description of Artificial Sequence Synthetic polypeptide”
MAEVQLVQSGGGLVKPGGSLRLSCAASGFTFSDYYMSWIRQAPGKGLEWVSYISSSGSTIYYADSVKGRFTISRDNSKNTLYLQMNSLRAEDTAVYYCARWSGEDAFDIWGQGTMVTVSSGGGGSGGGGSGGGGSDIVMTQSPSTLSASVGDRVAITCRASEGIYHWLAWYQQKPGKAPKLLIYKASSLASGAPSRFSGSGSGADFTLTISSLQPDDFATYYCQQYSNYPLTFGGGTKLEVKRAA

SEQ ID NO: 51 - protein, 245 aa, Artificial Sequence
  source: /note=“Description of Artificial Sequence Synthetic polypeptide”
MAEVQLVQSGGGVVQPGGSLSLSCAVSGITLRTYGMHWVRQAPGKGLEWVAGISFDGRSEYYADSVKGRFTISRDNSKNTLYLQMNSLRAEDTAVYYCARDRGSYGMDVWGRGTMVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEILRAA

SEQ ID NO: 52 - protein, 244 aa, Artificial Sequence
  source: /note=“Description of Artificial Sequence Synthetic polypeptide”
MAQVQLVQSGGGLVRPGGSLSLSCAVSGITLRTYGMHWVRQAPGKGLEWVAGISFDGRSEYYADSVQGRFTISRDSSKNTLYLQMNSLRAEDTAVYYCARGAHYGFDIWGQGTMVTVSSGGGGTGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTELEIKRAA

SEQ ID NO: 53 - protein, 245 aa, Artificial Sequence
  source: /note=“Description of Artificial Sequence Synthetic polypeptide”
  MOD_RES (208)..(208): Any amino acid
MAQVQLVESGGGLVKPGGSLRLSCAASGFTFSSHNMNWVRQAPGKGLEWVSSISSSSSYIYYADSVKGRFTISRDNAKNSLYLQMNSLRAEDTAVYYCARDRGSTGMDVWGRGTLVTVSSGGGGSGGGGSGGGGSKIQMTQSPSTLSASIGDRVTITCRASEGIYHWLAWYQQKPGKAPKLLIYKASSLASGAPSRFSGSGSGTDFTXTISSLQPDDFATYYCQQYSNYPLTFGGGTKLEIKRAA

SEQ ID NO: 54 - protein, 244 aa, Artificial Sequence
  source: /note=“Description of Artificial Sequence Synthetic polypeptide”
MAQVQLQQSGPGLVKPSETLSLTCTVSGDSISSYYWSWIRQPPGKGLEWIGYIYYSGSTNYNPSLKSRVTISVDTSKSQFSLKLSSVTAADTAVYYCARGRYFDVWGRGTMVTVSSGGGGSGGGGSGGGGSSYVLTQPPSVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLGAA

SEQ ID NO: 55 - protein, 266 aa, Artificial Sequence
  source: /note=“Description of Artificial Sequence Synthetic polypeptide”
MKHLWFFLLLVAAPRWVLSQVQLQQSGPGLVKPSETLSLTCTVSGDSISSYYWSWIRQPPGKGLEWIGYIYYSGSTNYNPSLKSRVTISVDTSKSQFSLKLSSVTAADTAVYYCARGRYFDVWGRGTMVTVSSGGGGSGGGGSGGGGSSYVLTQPPSVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLDYKDDDDK

SEQ ID NO: 56 - protein, 266 aa, Artificial Sequence
  source: /note=“Description of Artificial Sequence Synthetic polypeptide”
MEFGLSWVFLVALLRGVQCQVQLVQSGGGLVRPGGSLSLSCAVSGITLRTYGMHWVRQAPGKGLEWVAGISFDGRSEYYADSVQGRFTISRDSSKNTLYLQMNSLRAEDTAVYYCARGAHYGFDIWGQGTMVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKDYKDDDDK

SEQ ID NO: 57 - protein, 106 aa, Artificial Sequence
  source: /note=“Description of Artificial Sequence Synthetic polypeptide”
QIVLTQSPAIMSASPGEKVTISCSASSSVSYMYWYQQKPGSSPKPWIYRTSNLASGVPARFSGSGSGTSYSLTISNMEAEDAAAYYCQQYHSYPTTFGGGTKLEVK

SEQ ID NO: 58 - protein, 120 aa, Artificial Sequence
  source: /note=“Description of Artificial Sequence Synthetic polypeptide”
EVQLVESGGGLVQPKGSLKLSCAASGFSFNTYAMNWVRQAPGKGLEWIAHIRSKSNNFATYYADSVKDRFSISRDASENILFLQMNNLKTEDTAMYYCVRQGGDFPMDYWGQGTSVTVSS

SEQ ID NO: 59 - protein, 115 aa, Artificial Sequence
  source: /note=“Description of Artificial Sequence Synthetic polypeptide”
QVQLQQSGPELVKPGASVKMSCKASGYAFSSSWLNWVRQRPGKGLEWIGRIYPGDGENHYNGKFKGKATLTADKSSSTGYMQLSSLTSEDSAVYFCASYYEGGYWGQGTLITVSA

SEQ ID NO: 60 - protein, 112 aa, Artificial Sequence
  source: /note=“Description of Artificial Sequence Synthetic polypeptide”
DIVMTQAAPSIPVTPGESVSISCRSDKSLLHSNGNTYLFWFLQRPGQSPQLLIYRMSNLASGVPDRFSGSGSGTAFTLRISGVEAEDVGVYYCMQHLEYPYTFGGGTKLEIK

SEQ ID NO: 61 - protein, 118 aa, Artificial Sequence
  source: /note=“Description of Artificial Sequence Synthetic polypeptide”
DVQLQESGPGLVKPSQSLSLTCTVTGYSITIDYTWNWIRQFPGNKLEWMGYITYSGSTDYNPSLKSRSSITRDTSMNQFFLQLNSVTTEDTATYYCARLGRRYALDYWGQGTSVTVSS

SEQ ID NO: 62 - protein, 108 aa, Artificial Sequence
  source: /note=“Description of Artificial Sequence Synthetic polypeptide”
DIQMTQSSSSFSVSLGDRVTITCKASEDIYIRLAWYQQKPGNAPRLLISAATSLETGIPSRFSGSGSGEDYTLTITSLQTEDVATYYCQQYWTTPWTFGGGTKLEIKR

SEQ ID NO: 63 - protein, 113 aa, Artificial Sequence
  source: /note=“Description of Artificial Sequence Synthetic polypeptide”
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIKR

SEQ ID NO: 64 - protein, 114 aa, Artificial Sequence
  source: /note=“Description of Artificial Sequence Synthetic polypeptide”
QVQLQQSGPELVKPGASVKISCKASGYGFSNSWMNWVRQRPGKGLEWIGRIYPGDGETSYNGEFVGKATLTADKSSSTAYMHLSSLTSEDSAVYFCASYYEGGYWGQGTLVTVS

SEQ ID NO: 65 - protein, 113 aa, Artificial Sequence
  source: /note=“Description of Artificial Sequence Synthetic polypeptide”
DIVMTQAAPSLPVTPGESVSISCRSSKSLLHSNGNTYLFWFLQRPGQSPHLLIYRMSNLASGVPDRFSGSGSGTAFTLRISRVEAEDVGVYYCMQHLEYPYTFGGGTKLEIKR

SEQ ID NO: 66 - protein, 115 aa, Artificial Sequence
  source: /note=“Description of Artificial Sequence Synthetic polypeptide”
QVQLQQSGPELVKPGASVKISCKASGYGFSSSWMNWVKQRPGKGLEWIGRIYPGDGETSYNGEFKGKATLTADKSSSTAYMQLSSLTSEDSAVYFCASYYEGGYWGQGTLVTVSA

SEQ ID NO: 67 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 69 - protein, 103 aa, Unknown
  source: /note=“Description of Unknown CH1 sequence”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 70 - protein, 107 aa, Unknown [sequence omitted]

SEQ ID NO: 71 - protein, 106 aa, Unknown
  source: /note=“Description of Unknown CL (lambda) sequence”
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 72 - protein, 121 aa, Artificial Sequence
  source: /note=“Description of Artificial Sequence Synthetic polypeptide”
EVQLVESGGGLVQPGGSLRLSCAASGFSFSAGYWICWVRQAPGKGLEWIACTYAGRSGSTYYANWVNGRFTISKDSAKTSVYLQMNSLRAEDTAVYYCARGNAGVAVGALWGRGTLVTVSS

SEQ ID NO: 73 - protein, 121 aa, Artificial Sequence
  source: /note=“Description of Artificial Sequence Synthetic polypeptide”
EVQLVESGGGLVQPGGSLRLSCAASGFSFSAGYWISWVRQAPGKGLEWIASTYAGRSGSTYYANWVNGRFTISKDSAKTSVYLQMNSLRAEDTAVYYCARGNAGVAVGALWGRGTLVTVSS

SEQ ID NO: 74 - protein, 121 aa, Artificial Sequence
  source: /note=“Description of Artificial Sequence Synthetic polypeptide”
EVTLKESGPALVKPTQTLTLTCTASGFSFSAGYWICWVRQPPGKGLEWIACTYAGRSGSTYYANWVNGRFTISKDSSKTQVVLTMTNMDPVDTATYYCARGNAGVAVGALWGRGTLVTVSS

SEQ ID NO: 75 - protein, 121 aa, Artificial Sequence
  source: /note=“Description of Artificial Sequence Synthetic polypeptide”
EVTLKESGPALVKPTQTLTLTCTASGFSFSAGYWISWVRQPPGKGLEWIASTYAGRSGSTYYANWVNGRFTISKDSSKTQVVLTMTNMDPVDTATYYCARGNAGVAVGALWGRGTLVTVSS

SEQ ID NO: 76 - protein, 111 aa, Artificial Sequence
  source: /note=“Description of Artificial Sequence Synthetic polypeptide”
DIQMTQSPSTLSASVGDRVTITCQASQSISNWLAWYQQKPGKAPKLLIYQASKLASGVPSRFSGSGSGTEYTLTISSLQPDDFATYYCQSYYDSGSNVFFAFGGGTKVEIK

SEQ ID NO: 77 - protein, 121 aa, Artificial Sequence
  source: /note=“Description of Artificial Sequence Synthetic polypeptide”
EVQLVESGGGLVQPGGSLRLSCAASGFSFSAGYWICWVRQAPGKGLEWIACIYAGSSGSTYYASWAKGRFTISKDSAKTSVYLQMNSLRAEDTAVYYCARGNAGVAVGALWGRGTLVTVSS

SEQ ID NO: 78 - protein, 121 aa, Artificial Sequence
  source: /note=“Description of Artificial Sequence Synthetic polypeptide”
EVQLVESGGGLVQPGGSLRLSCAASGFSFSAGYWISWVRQAPGKGLEWIASIYAGSSGSTYYASWAKGRFTISKDSAKTSVYLQMNSLRAEDTAVYYCARGNAGVAVGALWGRGTLVTVSS

SEQ ID NO: 79 - protein, 121 aa, Artificial Sequence
  source: /note=“Description of Artificial Sequence Synthetic polypeptide”
EVTLKESGPALVKPTQTLTLTCTASGFSFSAGYWICWVRQPPGKGLEWIACIYAGSSGSTYYASWAKGRFTISKDSSKTQVVLTMTNMDPVDTATYYCARGNAGVAVGALWGRGTLVTVSS

SEQ ID NO: 80 - protein, 121 aa, Artificial Sequence
  source: /note=“Description of Artificial Sequence Synthetic polypeptide”
EVTLKESGPALVKPTQTLTLTCTASGFSFSAGYWISWVRQPPGKGLEWIASIYAGSSGSTYYASWAKGRFTISKDSSKTQVVLTMTNMDPVDTATYYCARGNAGVAVGALWGRGTLVTVSS

SEQ ID NO: 81 - protein, 111 aa, Artificial Sequence
  source: /note=“Description of Artificial Sequence Synthetic polypeptide”
DIQMTQSPSTLSASVGDRVTITCQASQSISSWLSWYQQKPGKAPKLLIYGASNLASGVPSRFSGSGSGTQFTLTISSLQPDDFATYYCQSYYDSGSSVFFNFGGGTKVEIK

SEQ ID NO: 82 - protein, 121 aa, Artificial Sequence
  source: /note=“Description of Artificial Sequence Synthetic polypeptide”
EVQLVESGGGLVQPGGSLRLSCAASGVSFSSSYWIYWVRQAPGKGLEWIACIYTGSSGSTYYASWAKGRFTVSEDSAKTSVYLQMNSLRAEDTAVYYCARASAWTYGMDLWGRGTLVTVSS

SEQ ID NO: 83 - protein, 121 aa, Artificial Sequence
  source: /note=“Description of Artificial Sequence Synthetic polypeptide”
EVQLVESGGGLVQPGGSLRLSCAASGVSFSSSYWIYWVRQAPGKGLEWIASIYTGSSGSTYYASWAKGRFTVSEDSAKTSVYLQMNSLRAEDTAVYYCARASAWTYGMDLWGRGTLVTVSS

SEQ ID NO: 84 - protein, 121 aa, Artificial Sequence
  source: /note=“Description of Artificial Sequence Synthetic polypeptide”
EVQLQESGPGLVKPSQTLSLTCTASGVSFSSSYWIYWVRQHPGKGLEWIACIYTGSSGSTYYASWAKGRFTVSEDSSKTQVSLKLSSVTAADTAVYYCARASAWTYGMDLWGRGTLVTVSS

SEQ ID NO: 85 - protein, 121 aa, Artificial Sequence
  source: /note=“Description of Artificial Sequence Synthetic polypeptide”
EVQLQESGPGLVKPSQTLSLTCTASGVSFSSSYWIYWVRQHPGKGLEWIASIYTGSSGSTYYASWAKGRFTVSEDSSKTQVSLKLSSVTAADTAVYYCARASAWTYGMDLWGRGTLVTVSS

SEQ ID NO: 86 - protein, 107 aa, Artificial Sequence
  source: /note=“Description of Artificial Sequence Synthetic polypeptide”
DIVMTQSPSSVSASVGDRVTITCQASQSFYNLLAWYQQKPGKAPKLLIYDASDLASGVPSRFKGSGSGTDFTLTISSLQPEDFATYYCQSADGSSYAFGGGTKVEIK

SEQ ID NO: 87 - protein, 107 aa, Artificial Sequence
  source: /note=“Description of Artificial Sequence Synthetic polypeptide”
DIQMTQSPSSVSASVGDRVTITCQASQSFYNLLAWYQQKPGKAPKLLIYDASDLASGVPSRFSGSGSGTDFTLTISSLQPEDFATYYCQSADGSSYAFGGGTKVEIK

SEQ ID NO: 88 - protein, 123 aa, Artificial Sequence
  source: /note=“Description of Artificial Sequence Synthetic polypeptide”
EVQLVESGGGLVKPGGSLRLSCAASGFSFSGNYYMCWVRQAPGKGLEWIGCLYTGSSGSTYYASWAKGRFTISKDSAKTSVYLQMNSLRAEDTAVYYCARDLGYEIDGYGGLWGQGTLVTVSS

SEQ ID NO: 89 - protein, 123 aa, Artificial Sequence
  source: /note=“Description of Artificial Sequence Synthetic polypeptide”
EVQLVESGGGLVKPGGSLRLSCAASGFSFSGNYYMSWVRQAPGKGLEWIGSLYTGSSGSTYYASWAKGRFTISKDSAKTSVYLQMNSLRAEDTAVYYCARDLGYEIDGYGGLWGQGTLVTVSS

SEQ ID NO: 90 - protein, 123 aa, Artificial Sequence
  source: /note=“Description of Artificial Sequence Synthetic polypeptide”
EVQLQESGPGLVKPSGTLSLTCAASGFSFSGNYYMCWVRQPPGKGLEWIGCLYTGSSGSTYYASWAKGRVTISKDSSKTQVSLKLSSVTAADTAVYYCARDLGYEIDGYGGLWGQGTLVTVSS

SEQ ID NO: 91 - protein, 123 aa, Artificial Sequence
  source: /note=“Description of Artificial Sequence Synthetic polypeptide”
EVQLQESGPGLVKPSGTLSLTCAASGFSFSGNYYMSWVRQPPGKGLEWIGSLYTGSSGSTYYASWAKGRVTISKDSSKTQVSLKLSSVTAADTAVYYCARDLGYEIDGYGGLWGQGTLVTVSS

SEQ ID NO: 92 - protein, 112 aa, Artificial Sequence
  source: /note=“Description of Artificial Sequence Synthetic polypeptide”
AQVLTQSPSSLSASVGDRVTITCQASQSVYNNNNLSWYQQKPGKAPKLLIYDASKLASGVPSRFSGSGSGTQFTLTISSLQPEDFATYYCLGGYYSSGWYFAFGGGTKVEIK

SEQ ID NO: 93 - protein, 112 aa, Artificial Sequence
  source: /note=“Description of Artificial Sequence Synthetic polypeptide”
AQVLTQSPSSLSASVGDRVTITCQASQSVYNNNNLAWYQQKPGKAPKLLIYDASKLASGVPSRFSGSGSGTQFTLTISSLQPEDFATYYCLGGYYSSGWYFAFGGGTKVEIK

SEQ ID NO: 94 - protein, 120 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGRTEVATPGAYWGQGTMVTVSS

SEQ ID NO: 95 - protein, 111 aa, Artificial Sequence
  source: /note=“Description of Artificial Sequence Synthetic polypeptide”
QAVLTQPSSVSGAPGQRVTISCTGSSSNIGAGYDVHWYQQLPGTAPKLLIYGNSNRPSGVPDRFSGSKSGTSASLAVTGLQAEDEADYYCQSYDSSLSDVVFGGGTKLTVL

SEQ ID NO: 96 - protein, 122 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYRDYGGNSHLFDYWGQGTTVTVSS

SEQ ID NO: 97 - protein, 107 aa, Artificial Sequence
  source: /note=“Description of Artificial Sequence Synthetic polypeptide”
EIVMTQSPSSLPASVGDRVTITCRASQNIKTYLHWYQQKPGKAPNLLIYAASNLQIGVPSRFSGSGSGTDFTLTISSLQPEDFATYFCQQSYITPPTFGQGTRLEIK

SEQ ID NO: 98 - protein, 118 aa, Artificial Sequence
  source: /note=“Description of Artificial Sequence Synthetic polypeptide”
GVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARRVISGAFDIWGQGTMVTVSS

SEQ ID NO: 99 - protein, 107 aa, Artificial Sequence
  source: /note=“Description of Artificial Sequence Synthetic polypeptide”
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 100 - protein, 123 aa, Artificial Sequence
  source: /note=“Description of Artificial Sequence Synthetic polypeptide”
QVQLVQSGAEVKKPGASVKVSCKASGYTFTSYYMHWVRQAPGQGLEWMGIINPSDGSTRYVEKFQGRVTMTRDTSTSTVYMELSSLRSEDTAVYFCARGMGPGPHYHFYMDVWGKGTMVTVSS

SEQ ID NO: 101 - protein, 108 aa, Artificial Sequence
  source: /note=“Description of Artificial Sequence Synthetic polypeptide”
SSELTQDPAVSVALGQTVRITCQGDSLRSYYTNWFQQKPGQAPLLVVYAKNKRPSGIPDRFSGSSSGNTASLTITGAQAEDEADYYCHSRDSGGNHVLFGGGTKLTVL

SEQ ID NO: 102 - protein, 125 aa, Artificial Sequence
  source: /note=“Description of Artificial Sequence Synthetic polypeptide”
QVQLVQSGAEVKKPGASVKVSCKASGYTFTGQYIHWVRQAPGQGLEWMGWISAYNGYTDYAQKVQGRVTMTTDTSTSTAYMELRSLRSDDTAVYYCAREVWPVAAADTFSVFDIWGRGTLVTVSS

SEQ ID NO: 103 - protein, 108 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 104 - protein, 118 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGTTGLHDSWGQGTMVTVSS

SEQ ID NO: 105 - protein, 111 aa, Artificial Sequence
  source: /note=“Description of Artificial Sequence Synthetic polypeptide”
QSVLTQPPSASGTPGQRVTISCSGSSSNVGSNFVYWYQQFPGTAPKLLIYRNNQRPSGVPDRFSGSKSGTSASLAISGLRSEDEADYYCAAWDDTLNGHYVFGGGTKLTVL

SEQ ID NO: 106 - protein, 118 aa, Artificial Sequence
  source: /note=“Description of Artificial Sequence Synthetic polypeptide”
EVQLVQSGAEVKKPGESLKISCKGYGYDFSRDWIAWVRQMPGKGLEWMGIIYPGDSDTRYSPSFEGQVTISADKSISTAYLQWRSLKASDTAMYYCARQRRLGWFDPWGQGTMVTVSS

SEQ ID NO: 107 - protein, 110 aa, Artificial Sequence
  source: /note=“Description of Artificial Sequence Synthetic polypeptide”
RSVLTQPPSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVL

SEQ ID NO: 108 - protein, 123 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHLPSGSSSSWAFDSWGRGTTVTVSS

SEQ ID NO: 109 - protein, 110 aa, Artificial Sequence
  source: /note=“Description of Artificial Sequence Synthetic polypeptide”
SYVLTQPPSASGTPGQRVTISCSGSSSNIGSNYVYWYQQLPGTAPKLLIYRNNQRPSGVPDRFSGSKSGTSASLAISGLQSEDEADYYCEAWDDNVDGPVFGGGTKLTVL

SEQ ID NO: 110 - protein, 121 aa, Artificial Sequence
  source: /note=“Description of Artificial Sequence Synthetic polypeptide”
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARDDTPTSDYGFDSWGQGTLVTVSS

SEQ ID NO: 111 - protein, 100 aa, Artificial Sequence
  source: /note=“Description of Artificial Sequence Synthetic polypeptide”
TQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKV

SEQ ID NO: 112 - protein, 136 aa, Unknown
  source: /note=“Description of Unknown TGF-beta receptor type II isoform 1 ECD sequence”
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 113 - protein, 161 aa, Unknown
  source: /note=“Description of Unknown TGF-beta receptor type II isoform 2 ECD sequence”
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 114 - protein, 272 aa, Unknown
  source: /note=“Description of Unknown TGF-beta receptor type II isoform 1 ECD dimer sequence”
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 115 - protein, 322 aa, Unknown
  source: /note=“Description of Unknown TGF-beta receptor type II isoform 2 ECD dimer sequence”
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 116 - protein, 93 aa, Unknown
  source: /note=“Description of Unknown TGF-beta receptor type I ECD sequence”
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 117 - protein, 5 aa, Artificial Sequence
  source: /note=“Description of Artificial Sequence Synthetic peptide”
GGGGS

SEQ ID NO: 118 - protein, 10 aa, Artificial Sequence
  source: /note=“Description of Artificial Sequence Synthetic peptide”
GGGGSGGGGS

SEQ ID NO: 119 - protein, 15 aa, Artificial Sequence
  source: /note=“Description of Artificial Sequence Synthetic peptide”
GGGGSGGGGSGGGGS

SEQ ID NO: 120 - protein, 17 aa, Artificial Sequence
  source: /note=“Description of Artificial Sequence Synthetic peptide”
DVPSGPGGGGGSGGGGS

(19) United States
(12) Patent Application Publication
(10) Pub. No.: US 2021/0246227 A1
(43) Pub. Date: Aug. 12, 2021

(54) MULTISPECIFIC MOLECULES THAT BIND TO MYELOPROLIFERATIVE LEUKEMIA (MPL) PROTEIN AND USES THEREOF

(71) Applicant: ELSTAR THERAPEUTICS, INC., Cambridge, MA (US)

(72) Inventors: Andreas Loew, Boston, MA (US); Brian Edward Vash, Cambridge, MA (US)

(21) Appl. No.: 16/616,714

(22) PCT Filed: May 31, 2018
(86) PCT No.: PCT/US2018/035440

§ 371 Date: Nov. 25, 2019

Related U.S. Application Data
(60) Provisional application No. 62/555,843, filed on Sep. 8, 2017.
(60) Provisional application No. 62/522,480, filed on Jun. 20, 2017.
(60) Provisional application No. 62/512,867, filed on May 31, 2017.

(51) Int. Cl. C07K 16/46; C07K 16/28

(52) U.S. Cl. CPC C07K 16/468; C07K 2317/522; C07K 16/2896; C07K 16/28; C07K 16/2809; C07K 16/2848; C07K 2317/31; C07K 2317/565; C07K 2317/76; C07K 2317/33; C07K 2317/55; C07K 2317/622; C07K 2317/524; C07K 2317/526; C07K 2317/35; C07K 2317/92; C07K 16/289

(57) ABSTRACT

Molecules that bind myeloproliferative leukemia (MPL) protein and methods of using the same are disclosed.

### RELATED APPLICATIONS

This application claims priority to U.S. Ser. No. 62/512,867 filed May 31, 2017, U.S. Ser. No. 62/522,480 filed Jun. 20, 2017, and U.S. Ser. No. 62/555,843 filed Sep. 8, 2017, the content of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 24, 2018, is named E2070-7010WO_SL.txt and is 150,404 bytes in size.

### BACKGROUND

Myeloproliferative leukemia (MPL) protein, also known as thrombopoietin receptor (TPOR) or CD110 (Cluster of Differentiation 110), is a dimeric protein that is activated by the binding of its natural ligand, thrombopoieitin (TPO), to the extracellular domains (ECD) of MPL. Upon ligand binding, dimerization of the MPL receptor is induced, which results in a conformational change in MPL that is followed by activation of intracellular processing signals, including intracellular phosphorylation and activation of the JAK2 kinase pathway.

MPL agonists are known in the art, which are believed to function by bridging two receptor extracellular domains (ECD) of MPL proteins. However, the need still exits for MPL antagonists, such as the ones disclosed herein.

### SUMMARY OF THE INVENTION

Provided herein are, inter alia, multispecific molecules (e.g., a multispecific or multifunctional antibody molecules), comprising a first MPL-targeting moiety, wherein the first MPL-targeting moiety binds to MPL. In some embodiments, the multispecific molecule reduces, e.g., inhibits, an MPL activity. In some embodiments, the multispecific molecule comprises a second MPL-targeting moiety that binds to MPL. In some embodiments, the first and the second MPL-targeting moieties bind the same epitope (e.g., bind overlapping epitopes). In some embodiments, the binding of the first MPL-targeting moiety to a first MPL protein reduces (e.g., prevents) the binding of the second MPL-targeting moiety to a second MPL protein. In some embodiments, the first and the second MPL-targeting moieties bind different epitopes on a single MPL protein (e.g., bind non overlapping epitopes). In some embodiments, the multispecific molecule is a biparatopic antibody molecule (e.g., it binds to two different epitopes (e.g., non-overlapping epitopes) on the same MPL protein).

In embodiments, the multispecific molecule can further comprise one or more of an immune cell engager, a cytokine molecule, or a tumor targeting molecule (e.g., a second tumor targeting molecule that targets a tumor target other than MPL). In embodiments, the multispecific molecule, comprises a tumor targeting molecule wherein the tumor targeting molecule is an anti-CD41 antibody molecule or an anti-CD177 antibody molecule. In some embodiments, the multispecific molecule further comprises an anti-PDL1 antibody molecule, an anti-CD3 antibody molecule, an anti-TGFβ antibody molecule, a TGFβtrap polypeptide (e.g., a polypeptide comprising a portion of TGFβ receptor that is capable of binding TGFβ), an anti-IL1β antibody molecule, an IL1β trap polypeptide (e.g., a polypeptide comprising a portion of IL1β receptor that is capable of binding IL1β), an anti-CXCL10 antibody molecule, an anti-MS4A3 antibody molecule, an anti-OLFM4 antibody molecule, an anti-CD66b antibody molecule, an anti-cKit antibody molecule, an anti-FLT3 antibody molecule, or an anti-CD133 antibody molecule (or any combination thereof).

In another embodiment, the multispecific molecule or the MPL-binding molecule is or comprises a single MPL-targeting moiety, e.g., a half-arm antibody against MPL (e.g., a Fab or single chain Fv fused to a first immunoglobulin constant domain (e.g., a first Fc constant region (e.g., a first CH2-CH3)). Optionally, the half-arm antibody is dimerized (e.g., homo- or heterodimerized) to a second immunoglobulin constant domain, e.g., a second heavy chain constant region, e.g., a second Fc constant region, e.g., a second CH2-CH3). In embodiments, the MPL targeting moiety and/or the second immunoglobulin constant domain can further comprise one or more of an immune cell engager, a cytokine molecule, or a tumor targeting molecule (e.g., a second tumor targeting molecule that targets a tumor target other than MPL). Additionally disclosed are nucleic acids encoding the aforesaid multispecific molecules, methods of producing the aforesaid molecules, and methods of treating a cancer using the aforesaid molecules.

Accordingly, in one aspect, the disclosure features a multispecific or multifunctional molecule (e.g., multispecific or multifunctional polypeptide) that binds to (e.g., targets) MPL, e.g., that binds to one or more regions of MPL. In embodiments, the multispecific molecule includes a moiety, e.g., an antibody molecule or a ligand, that binds to MPL (also referred to herein as an “MPL targeting moiety”). In one embodiment, the multispecific molecule binds to the extracellular domain of MPL. In some embodiments, the multispecific molecule disclosed herein binds to a single MPL protein and prevents the association with a second MPL protein. In embodiments, the multispecific molecule reduces, e.g., inhibits, an MPL activity, e.g., reduces (e.g., prevents) one, two or more of MPL protein dimerization, intracellular phosphorylation or activation of the JAK2 kinase pathway. In embodiments, the MPL activity is reduced in the presence of an MPL ligand, e.g., TPO. In some embodiments, the multispecific molecule blocks binding of TPO to MPL.

In embodiments, the multispecific molecule binds to two or more epitopes (e.g., two or more non-overlapping epitopes) on a single MPL protein (e.g., the same MPL protein).

In one embodiment, the multispecific molecule includes two MPL-targeting moieties, e.g., a first MPL-targeting moiety that binds to a first epitope on the extracellular domain of the MPL protein, and a second MPL-targeting moiety that binds to a second epitope on the extracellular domain of the same MPL protein. In some embodiments, the first and second epitopes are different. In some embodiments, the two MPL targeting moieties, e.g., the first and second MPL-targeting moieties, bind to different epitopes on the extracellular domain of MPL protein. In one embodiment, the multispecific molecule binds to two different epitopes (e.g., non-overlapping epitopes) on the same MPL protein, e.g., is or comprises a biparatopic molecule. In some embodiments, the multispecific molecule comprises two binding specificities or functions, e.g., it is a bispecific or a bifunctional molecule.

In some embodiments of any of the multispecific molecules disclosed herein, the affinity, e.g., the combined affinity, for the MPL of the first and the second MPL-targeting moieties is equal to or greater than the affinity of each targeting moiety (either alone or as part of the multispecific molecule) for its corresponding antigen binding site. For example, the affinity, e.g., the combined affinity, for the MPL of the first and the second MPL-targeting moieties is at least 2, 5, 10, 20, 30, 40, 50, 75 or 100 times greater than the affinity of each targeting moiety (either alone or as part of the multispecific molecule) for its corresponding antigen binding site.

In yet other embodiments of any of the aforesaid multispecific molecules, the affinity, e.g., the combined affinity, of the first and the second MPL-targeting moieties for an MPL protein expressing cell, e.g., a cancer cell or a hematopoietic cell, is equal to or greater than the affinity of a similar multispecific or multifunctional molecule having only one of the first MPL-targeting moiety or the second MPL-targeting moiety. For example, the affinity, e.g., the combined affinity, of the first and the second MPL-targeting moieties for the MPL protein expressing cell, e.g., a cancer cell or a hematopoietic cell, is at least 2, 5, 10, 20, 30, 40, 50, 75 or 100 times greater than the affinity of a similar multispecific or multifunctional molecule having only one of the first MPL-targeting moiety or the second MPL-targeting moiety.

In yet other embodiments of any of the aforesaid multispecific molecules, the affinity, e.g., the combined affinity, of the first and the second MPL-targeting moieties for an MPL protein expressing cell, e.g., a cancer cell or a hematopoietic cell, is equal to or greater than the affinity of a ligand, e.g., a natural ligand of an MPL protein (e.g., TPO), for the MPL protein expressing cell, e.g., a cancer cell or a hematopoietic cell. For example, the affinity, e.g., the combined affinity, of the first and the second MPL-targeting moieties for the MPL protein expressing cell, e.g., a cancer cell or a hematopoietic cell, is at least 2, 5, 10, 20, 30, 40, 50, 75 or 100 times greater than the affinity of the ligand, e.g., a natural ligand of an MPL protein (e.g., TPO), for the MPL protein expressing cell, e.g., a cancer cell or a hematopoietic cell.

In other embodiments, the multispecific molecule includes a single MPL-targeting moiety, e.g., a half-arm antibody against MPL. In some embodiments, the multispecific molecule includes a Fab linked to, e.g., fused to, an immunoglobulin constant domain (e.g., a first Fc constant region (e.g., a first CH2-CH3)). Optionally, the half-arm antibody is dimerized (e.g., homo- or heterodimerized) to a second immunoglobulin constant domain, e.g., a second heavy chain constant region, e.g., a second Fc constant region, e.g., a second CH2-CH3.

In some embodiments, the multispecific molecule includes an MPL-targeting antibody molecule that binds to the MPL antigen with a dissociation constant of less than about 10 nM, and more typically, 10-100 pM.

In some embodiments, the multispecific molecule includes an MPL-targeting antibody molecule that binds to a conformational or a linear epitope on the MPL antigen.

In some embodiments, the multispecific molecule includes an MPL-targeting antibody molecule that is a monospecific antibody molecule, a bispecific antibody molecule, or a trispecific antibody molecule.

In some embodiments, the multispecific molecule includes an MPL-targeting antibody molecule that is a monovalent antibody molecule, a bivalent antibody molecule, or a trivalent antibody molecule.

In some embodiments, the multispecific molecule includes an MPL-targeting antibody molecule that is a biparatopic antibody molecule or a triparatopic antibody molecule, e.g., binds to two or three different epitopes on the same MPL molecule.

In some embodiments, the MPL targeting antibody molecule is a full-length antibody (e.g., an antibody that includes at least one, and preferably two, complete heavy chains, and at least one, and preferably two, complete light chains), or an antigen-binding fragment (e.g., a Fab, F(ab′)2, Fv, a single chain Fv, a single domain antibody, a half-arm antibody, a diabody (dAb), a bivalent antibody, a monovalent antibody, or a bispecific antibody or fragment thereof, a single domain variant thereof, or a camelid antibody).

In some embodiments, the multispecific molecule, e.g., the MPL targeting antibody molecule, comprises a heavy chain constant region chosen from IgG1, IgG2, IgG3, or IgG4, or a fragment thereof.

In some embodiments, the multispecific molecule, e.g., the MPL targeting antibody molecule, comprises a light chain constant region chosen from the light chain constant regions of kappa or lambda, or a fragment thereof.

In some embodiments, the multispecific molecule further comprises an immunoglobulin constant region (e.g., Fc region) chosen from the heavy chain constant regions of IgG1, IgG2, and IgG4, more particularly, the heavy chain constant region of human IgG1, IgG2 or IgG4. In some embodiments, the immunoglobulin constant region (e.g., an Fc region) is linked, e.g., covalently linked to, the MPL-targeting moiety.

In some embodiments, the immunoglobulin constant region (e.g., an Fc region) is linked, e.g., covalently linked to two MPL-targeting moieties, e.g., MPL-targeting moieties with non-overlapping antigen binding sites. In some embodiments, the immunoglobulin chain constant region (e.g., Fc region) is engineered, e.g., mutated, to increase or decrease one or more of: Fc receptor binding, antibody glycosylation, the number of cysteine residues, effector cell function, or complement function.

In some embodiments, the multispecific molecule comprises an MPL-targeting moiety that comprises a light chain constant region chosen from the light chain constant region of kappa or lambda, or a fragment thereof.

In some embodiments, the multispecific molecule comprises a first MPL-targeting moiety and a second MPL-targeting moiety, wherein the first MPL-targeting moiety comprises a kappa light chain constant region, or a fragment thereof, and the second MPL-targeting moiety comprises a lambda light chain constant region, or a fragment thereof.

In some embodiments, the multispecific molecule comprises a first MPL moiety and a second MPL-targeting moiety, wherein the first MPL-targeting moiety and the second MPL-targeting moiety comprise a common light chain variable region.

In some embodiments, the multispecific molecule comprises a dimerization domain, e.g., an interface of a first and second immunoglobulin chain constant regions (e.g., Fc region). In embodiments, the dimerization domain is engineered, e.g., mutated, to increase or decrease dimerization, e.g., relative to a non-engineered interface. In embodiments, the dimerization domain is engineered, e.g., mutated, to increase dimerization, e.g., relative to a non-engineered interface.

In some embodiments, the dimerization of the immunoglobulin chain constant regions (e.g., Fc regions) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

In some embodiments, the immunoglobulin chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1.

In some embodiments, the immunoglobulin chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof.

In some embodiments, the multispecific molecule comprises at least two, e.g., at least two, three or four, non-contiguous polypeptide chains. In some embodiments, the multispecific or multifunctional MPL antagonist molecule comprises two non-contiguous polypeptide chains. In some embodiments, the multispecific or multifunctional MPL antagonist molecule comprises three non-contiguous polypeptide chains. In some embodiments, the multispecific or multifunctional MPL antagonist molecule comprises four non-contiguous polypeptide chains.

In some embodiments, the multispecific molecule comprises the following non-contiguous polypeptides:

i) a first polypeptide which comprises, e.g., in the N- to C-orientation, an MPL-targeting moiety, e.g., an antibody molecule (e.g., a first portion of a first antigen domain, e.g., a first VH-CH1 of a Fab molecule) that binds to, e.g., an MPL antigen, and optionally, a domain that promotes association of the first and third polypeptide, e.g., an Fc molecule;

ii) a second polypeptide which comprises, e.g., in the N- to C-orientation, an MPL-targeting moiety, e.g., an antibody molecule (e.g., a second portion of a first antigen domain, e.g., a first VL-CL of a Fab molecule) that binds to, e.g., an MPL antigen (e.g., the same antigen bound by the first VH-CH1);

iii) a third polypeptide which comprises, e.g., in the N- to C-orientation, an MPL-targeting moiety, e.g., an antibody molecule (e.g., a first portion of a second antigen domain, e.g., a first VH-CH1 of a Fab molecule) that binds to, e.g., an MPL antigen, and optionally, a domain that promotes association of the first and third polypeptide, e.g., an Fc molecule; and

iv) a fourth polypeptide which comprises, e.g., in the N- to C-orientation, an MPL-targeting moiety, e.g., an antibody molecule (e.g., a second portion of a second antigen domain, e.g., a first VL-CL of a Fab molecule) that binds to, e.g., an MPL antigen (e.g., the same antigen bound by the second VH-CH1.

In some embodiments, the multispecific molecule comprises the following non-contiguous polypeptides:

i) a first polypeptide which comprises, e.g., in the N- to C-orientation, an MPL-targeting moiety, e.g., an antibody molecule (e.g., a first portion of a first antigen domain, e.g., a first VH of a scFv molecule) that binds to, e.g., an MPL antigen, and a second portion of a first antigen domain, e.g., a first VL of a scFv molecule) that also binds to, e.g., an MPL antigen (e.g., the same antigen bound by the first VH), and optionally, a domain that promotes association of the first and second polypeptide, e.g., an Fc molecule;

ii) a second polypeptide which comprises, e.g., in the N- to C-orientation, an MPL-targeting moiety, e.g., an antibody molecule (e.g., a second portion of a second antigen domain, e.g., a second VH of a scFv molecule) that binds to, e.g., an MPL antigen, and a second portion of a second antigen domain, e.g., a second VL of a scFv molecule) that also binds to, e.g., an MPL antigen (e.g., the same antigen bound by the second VH), and optionally, a domain that promotes association of the first and second polypeptide, e.g., an Fc molecule.

In some embodiments, the multispecific molecule comprises the following non-contiguous polypeptides:

i) a first polypeptide which comprises, e.g., in the N- to C-orientation, an MPL-targeting moiety, e.g., an antibody molecule (e.g., a first portion of a first antigen domain, e.g., a first VH-CH1 of a Fab molecule) that binds to, e.g., an MPL antigen, and optionally, a domain that promotes association of the first and third polypeptide, e.g., an Fc molecule;

ii) a second polypeptide which comprises, e.g., in the N- to C-orientation, an MPL-targeting moiety, e.g., an antibody molecule (e.g., a second portion of a first antigen domain, e.g., a first VL-CL of a Fab molecule) that binds to, e.g., an MPL antigen (e.g., the same antigen bound by the first VH-CH1); and

iii) a third polypeptide which comprises, e.g., in the N- to C-orientation, a domain that promotes association of the first and third polypeptides, e.g., an Fc molecule.

In some embodiments, the multispecific molecule comprises the following non-contiguous polypeptides:

i) a first polypeptide which comprises, e.g., in the N- to C-orientation, an MPL-targeting moiety, e.g., an antibody molecule (e.g., a first portion of a first antigen domain, e.g., a first VH of a scFv molecule) that binds to, e.g., an MPL antigen, and a second portion of a first antigen domain, e.g., a first VL of a scFv molecule) that also binds to, e.g., an MPL antigen (e.g., the same antigen bound by the first VH), and optionally, a domain that promotes association of the first and second polypeptide, e.g., an Fc molecule.

In one aspect, disclosed herein is a multispecific molecule comprising a first antigen-binding domain and a second antigen-binding domain, wherein the first and the second antigen-binding domains bind different epitopes on a single MPL protein (e.g., bind non overlapping epitopes), and wherein the first antigen-binding domain comprises a first polypeptide and a second polypeptide, and the second antigen-binding domain comprises a third polypeptide and a fourth polypeptide, wherein:

a) the first polypeptide comprises, e.g., in the N- to C-orientation, a first heavy chain variable region (VH), a first heavy chain constant region 1 (CH1), and optionally a first region that promotes association of the first and third polypeptides, e.g., a first Fc region (e.g., a first CH2-CH3);

b) the second polypeptide comprises, e.g., in the N- to C-orientation, a first light chain variable region (VL) and a first light chain constant region (CL);

c) the third polypeptide comprises, e.g., in the N- to C-orientation, a second heavy chain variable region (VH), a second heavy chain constant region 1 (CH1), and optionally, a second region that promotes association of the first and third polypeptides, e.g., a second Fc region (e.g., a second CH2-CH3); and

d) the fourth polypeptide comprises, e.g., in the N- to C-orientation, a second light chain variable region (VL) and a second light chain constant region (CL).

In one aspect, disclosed herein is a multispecific molecule comprising a first antigen-binding domain and a second antigen-binding domain, wherein the first antigen-binding domain binds to MPL and the second antigen-binding domain binds to an antigen other than MPL, e.g., a tumor antigen other than MPL, and wherein the first antigen-binding domain comprises a first polypeptide and a second polypeptide, and the second antigen-binding domain comprises a third polypeptide and a fourth polypeptide, wherein:

a) the first polypeptide comprises, e.g., in the N- to C-orientation, a first heavy chain variable region (VH), a first heavy chain constant region 1 (CH1), and optionally a first region that promotes association of the first and third polypeptides, e.g., a first Fc region (e.g., a first CH2-CH3);

b) the second polypeptide comprises, e.g., in the N- to C-orientation, a first light chain variable region (VL) and a first light chain constant region (CL);

c) the third polypeptide comprises, e.g., in the N- to C-orientation, a second heavy chain variable region (VH), a second heavy chain constant region 1 (CH1), and optionally, a second region that promotes association of the first and third polypeptides, e.g., a second Fc region (e.g., a second CH2-CH3); and

d) the fourth polypeptide comprises, e.g., in the N- to C-orientation, a second light chain variable region (VL) and a second light chain constant region (CL).

In one aspect, disclosed herein is a multispecific molecule comprising a first antigen-binding domain and a second antigen-binding domain, wherein the first and the second antigen-binding domains bind different epitopes on a single MPL protein (e.g., bind non overlapping epitopes), and wherein the first antigen-binding domain comprises a first polypeptide, and the second antigen-binding domain comprises a second polypeptide, wherein:

a) the first polypeptide comprises, e.g., in the N- to C-orientation, a first scFv region comprising a first heavy chain variable region (VH) and a first light chain variable region (VL), and optionally, a first region that promotes association of the first and second polypeptides, e.g., a first Fc region (e.g., a first CH2-CH3);

b) the second polypeptide comprises, e.g., in the N- to C-orientation, a second scFv region comprising a second VH and a second VL, and optionally, a second region that promotes association of the first and second polypeptides, e.g., a second Fc region (e.g., a second CH2-CH3).

In one aspect, disclosed herein is a multispecific molecule comprising a first antigen-binding domain and a second antigen-binding domain, wherein the first antigen-binding domain binds to MPL and the second antigen-binding domain binds to an antigen other than MPL, e.g., a tumor antigen other than MPL, and wherein the first antigen-binding domain comprises a first polypeptide, and the second antigen-binding domain comprises a second polypeptide, wherein:

a) the first polypeptide comprises, e.g., in the N- to C-orientation, a first scFv region comprising a first heavy chain variable region (VH) and a first light chain variable region (VL), and optionally, a first region that promotes association of the first and second polypeptides, e.g., a first Fc region (e.g., a first CH2-CH3);

b) the second polypeptide comprises, e.g., in the N- to C-orientation, a second scFv region comprising a second VH and a second VL, and optionally, a second region that promotes association of the first and second polypeptides, e.g., a second Fc region (e.g., a second CH2-CH3).

In one aspect, disclosed herein is an MPL-binding molecule comprising:

i) a single MPL-targeting moiety comprising a first polypeptide and a second polypeptide; and

ii) a third polypeptide, wherein:

a) the first polypeptide comprises, e.g., in the N- to C-orientation, a heavy chain variable region (VH) and a heavy chain constant region 1 (CH1), and optionally, a first region that promotes association of the first and third polypeptides, e.g., a first Fc region (e.g., a first CH2-CH3);

b) the second polypeptide comprises, e.g., in the N- to C-orientation, a light chain variable region (VL) and a light chain constant region (CL); and

c) the third polypeptide comprises, e.g., in the N- to C-orientation, a second region that promotes association of the first and third polypeptides, e.g., a second Fc region (e.g., a second CH2-CH3).

In one aspect, disclosed herein is an MPL-binding molecule comprising a single MPL-targeting moiety comprising an scFv comprising a heavy chain variable region (VH) and a light chain variable region (VL), wherein:

i) the MPL-binding molecule further comprises an immunoglobulin constant domain, e.g., an Fc constant region, e.g., a CH2-CH3; and/or

ii) the MPL-binding molecule reduces, e.g., inhibits, an MPL activity.

In one aspect, disclosed herein is a multispecific molecule or MPL-binding molecule which binds preferentially to an MPL associated with a mutated JAK2 (e.g., a JAK2 comprising a V617F mutation) over an MPL associated with a wild-type JAK2. In some embodiments, the multispecific molecule or MPL-binding molecule binds to an MPL associated with a mutated JAK2 (e.g., a JAK2 comprising a V617F mutation) with a greater affinity, e.g., at least 2, 5, 10, 20, 30, 40, 50, 75 or 100 times greater affinity, than when the multispecific molecule or MPL-binding molecule binds to an MPL associated with a wild-type JAK2. In some embodiments, the multispecific molecule or MPL-binding molecule binds to an epitope that is only present in MPL when MPL is associated with a mutated JAK2 (e.g., a JAK2 comprising a V617F mutation), but not when MPL is associated with a wild-type JAK2.

### Exemplary MPL-Targeting Moieties

In one embodiment, the MPL-targeting moiety includes an antibody molecule (e.g., Fab or scFv) that binds to MPL. In some embodiments, the antibody molecule to MPL comprises one, two, or three CDRs from any of the heavy chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 1. In some embodiments, the antibody molecule to MPL comprises a heavy chain variable domain sequence chosen from any of the heavy chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

Alternatively, or in combination with the heavy chain to MPL disclosed herein, the antibody molecule to MPL comprises one, two, or three CDRs from any of the light chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 1. In some embodiments, the antibody molecule to MPL comprises a light chain variable domain sequence chosen from any of the light chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

In one embodiment, the MPL-targeting moiety includes an antibody molecule (e.g., Fab or scFv) that binds to MPL. In some embodiments, the antibody molecule to MPL comprises one, two, or three CDRs from the heavy chain variable domain sequence of SEQ ID NO: 1 (see Table 1), or a closely related CDR, e.g., a CDR which has at least one amino acid alteration, but not more than two, three or four alterations (e.g., substitutions, deletions, or insertions, e.g., conservative substitutions) from the CDR sequence of SEQ ID NO: 1.

In embodiments, the antibody molecule to MPL includes the heavy chain variable domain sequence of SEQ ID NO: 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 1.

In embodiments, the antibody molecule to MPL is a Fab and further comprises a heavy chain constant region (CH1) having the amino acid sequence of SEQ ID NO:69 (see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 69.

Alternatively, or in combination with the heavy chain to MPL disclosed herein, the antibody molecule to MPL comprises one, two, or three CDRs from the light chain variable domain sequence of SEQ ID NO: 2 (see Table 1), or a closely related CDR, e.g., a CDR which has at least one amino acid alteration, but not more than two, three or four alterations (e.g., substitutions, deletions, or insertions, e.g., conservative substitutions) from the CDR sequence of SEQ ID NO: 2.

In some embodiments, the antibody molecule to MPL comprises the light chain variable domain sequence of SEQ ID NO: 2, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 2.

In some embodiments, the antibody molecule to MPL is a Fab and further comprises a light chain constant region (CL(kappa)) having the amino acid sequence SEQ ID NO: 70 (see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 70.

In some embodiments, the antibody molecule to MPL is a Fab and further comprises a light chain constant region (CL(lambda)) having the amino acid sequence SEQ ID NO: 71(see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 71.

In other embodiments, the antibody molecule to MPL comprises one, two, or three CDRs from the heavy chain variable domain, and one, two, or three CDRs from the light chain variable domain sequence of any of the pairs of variable domains presented in Table 1, e.g., variable heavy and variable light domains from the same antibody molecule, e.g., variable heavy domain of SEQ ID NO: 3 and variable light domain of SEQ ID NO:4.

In embodiments, the antibody molecule to MPL includes the heavy chain variable domain sequence of SEQ ID NO: 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 3, and the light chain variable domain sequence of SEQ ID NO: 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 4.

In embodiments, the antibody molecule to MPL is a single chain Fv comprising the sequence of SEQ ID NO: 49, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 49.

In embodiments, the antibody molecule further comprises:

(i) a heavy chain constant region (CH1) having the amino acid sequence of SEQ ID NO: 69 (see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 69;

(ii) a light chain constant region (CL(kappa)) having the amino acid sequence SEQ ID NO: 70 (see Table 2); or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 70; or

(iii) a light chain constant region (CL(lambda)) having the amino acid sequence SEQ ID NO: 71(see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 71.

In embodiments, the antibody molecule to MPL is a single chain Fv comprising any of SEQ ID NOs: 50-56, or amino acid sequences substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequences of SEQ ID NOs: 50-56.

Multispecific Molecules that Bind to MPL and a Phosphatase

Deregulation of the JAK/STAT pathway is a key-mediator of tumor genesis in a number of hematological tumors including myelofibrosis. CD45 negatively regulates the JAK/STAT pathway downstream of MPL. In one aspect, the present invention discloses a multispecific molecule that co-ligates CD45 with MPL (e.g., a multispecific molecule comprising a first targeting moiety that binds to MPL and a second targeting moiety that binds to CD45). Without wishing to be bound by theory, such a multispecific molecule can enhance the de-phosphorylation of JAK2 kinase anchored to the inside of the MPL intracellular domain and thus down regulate the MPL/JAK2 pathway in malignant cells.

In one aspect, disclosed herein is a multispecific molecule comprising a first targeting moiety that binds to MPL and a second targeting moiety that binds to a phosphatase, e.g., a protein tyrosine phosphatase (PTP), e.g., a receptor protein tyrosine phosphatase (RPTP). In one embodiment, the phosphatase and MPL are expressed in a same cell, e.g., a myelofibrosis cell.

In one embodiment, the phosphatase (e.g., a receptor protein tyrosine phosphatase (RPTP)) can modulate the MPL/JAK2 pathway. In one embodiment, the phosphatase can dephosphorylate MPL or a molecule that interacts directly or indirectly with MPL (e.g., a tyrosine kinase that interacts directly or indirectly with MPL, e.g., JAK2 or Src). Without wishing to be bound by theory, when the phosphatase (e.g., a protein tyrosine phosphatase (PTP), e.g., a receptor protein tyrosine phosphatase (RPTP), e.g., CD45, CD148, or LAR) is brought in close proximity to MPL by the multispecific molecule disclosed herein, the phosphatase dephosphorylates MPL or a molecule that interacts directly or indirectly with MPL (e.g., a tyrosine kinase that interacts directly or indirectly with MPL, e.g., JAK2 or Src), thereby negatively regulating down-stream signaling pathways.

In one embodiment, the phosphatase is selected from the group consisting of CD45, RPTPμ, RPTPκ, RPTPρ, RPTPλ, leukocyte antigen-related tyrosine phosphatase (LAR), RPTPσ, RPTPδ, RPTPβ, CD148, SAP1, RPTPO, RPTPQ/PTPS31, RPTPα, RPTPε, RPTPζ, RPTPγ, PC-PTP, IA2, and IA2β. In one embodiment, the phosphatase is CD45. In one embodiment, the phosphatase is CD148. In one embodiment, the phosphatase is leukocyte antigen-related tyrosine phosphatase (LAR).

In one embodiment, the second targeting moiety binds to an extracellular domain of CD45. In one embodiment, the second targeting moiety binds to one or more of CD45RA, CD45RB, CD45RC, CD45RAB, CD45RAC, CD45RBC, CD45RO, or CD45R (ABC). In one embodiment, the second targeting moiety binds specifically to one CD45 isoform. In one embodiment, the second targeting moiety binds to more than one CD45 isoform. In one embodiment, the second targeting moiety binds to all CD45 isoforms. In one embodiment, the second targeting moiety binds to a CD45 isoform that is expressed in a myelofibrosis cell. In one embodiment, the second targeting moiety binds to a CD45 isoform that is expressed by a same cell as MPL is.

In one embodiment, the first targeting moiety that binds to MPL comprises:

(i) one, two, or three CDRs from any of the heavy chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 1;

(ii) a heavy chain variable domain sequence chosen from any of the heavy chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);

(iii) one, two, or three CDRs from any of the light chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 1; or

(iv) a light chain variable domain sequence chosen from any of the light chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

In one embodiment, the second targeting moiety that binds to a phosphatase binds to CD45, wherein the second targeting moiety comprises:

(i) one, two, or three CDRs from any of the heavy chain variable domain sequences of Table 3, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 3;

(ii) a heavy chain variable domain sequence chosen from any of the heavy chain variable domain amino acid sequences of Table 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);

(iii) one, two, or three CDRs from any of the light chain variable domain sequences of Table 3, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 3; or

(iv) a light chain variable domain sequence chosen from any of the light chain variable domain amino acid sequences of Table 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

In one embodiment, the second targeting moiety that binds to a phosphatase binds to CD148, wherein the second targeting moiety comprises:

(i) one, two, or three CDRs from any of the heavy chain variable domain sequences of Table 4, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 4;

(ii) a heavy chain variable domain sequence chosen from any of the heavy chain variable domain amino acid sequences of Table 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);

(iii) one, two, or three CDRs from any of the light chain variable domain sequences of Table 4, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 4; or

(iv) a light chain variable domain sequence chosen from any of the light chain variable domain amino acid sequences of Table 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

In one embodiment, the second targeting moiety that binds to a phosphatase binds to LAR, wherein the second targeting moiety comprises:

(i) one, two, or three CDRs from any of the heavy chain variable domain sequences of Table 5, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 5;

(ii) a heavy chain variable domain sequence chosen from any of the heavy chain variable domain amino acid sequences of Table 5, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);

(iii) one, two, or three CDRs from any of the light chain variable domain sequences of Table 5, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 5; or

(iv) a light chain variable domain sequence chosen from any of the light chain variable domain amino acid sequences of Table 5, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

### Additional Moieties

The multispecific molecules disclosed herein can further include one or more of an immune cell engager, a cytokine molecule, a cytokine antagonist, e.g., a TGF-β antagonist, a stromal modifier, an enzyme, a toxin, a labeling agent, or a tumor targeting molecule (e.g., a second tumor targeting molecule that targets a tumor target other than MPL).

In one aspect, provided herein is a multispecific molecule that includes:

(i) two MPL-targeting moieties, e.g., a first MPL-targeting moiety that binds to a first epitope on the extracellular domain of the MPL protein, and a second MPL-targeting moiety that binds to a second epitope on the extracellular domain of the MPL protein, wherein the first and second epitopes are non-overlapping; and

(ii) any two or a combination thereof chosen from: an immune cell engager, e.g., an NK cell engager, a T cell engager, a B cell engager, a dendritic cell engager, or a macrophage cell engager; or a cytokine molecule, e.g., that includes at least two non-contiguous polypeptides (e.g., a multichain cytokine), e.g., the cytokine molecule comprises two chains, e.g., an alpha and beta chain (e.g., IL-12); or a tumor targeting molecule (e.g., a second tumor targeting molecule that targets a tumor target other than MPL).

In one aspect, provided herein is a multispecific molecule that comprises:

(i) a first targeting moiety that binds to MPL;

(ii) a second targeting moiety that binds to a phosphatase, e.g., a protein tyrosine phosphatase (PTP), e.g., a receptor protein tyrosine phosphatase (RPTP), e.g., CD45, CD148, or LAR; and

(iii) an immune cell engager, e.g., a T cell engager, e.g., an anti-CD3 antibody molecule.

In one aspect, provided herein is a multispecific molecule that comprises:

(i) a first targeting moiety that binds to MPL;

(ii) a second targeting moiety that binds to a phosphatase, e.g., a protein tyrosine phosphatase (PTP), e.g., a receptor protein tyrosine phosphatase (RPTP), e.g., CD45, CD148, or LAR; and

(iii) a TGF-β antagonist, e.g., a polypeptide comprising a TGFβ receptor, or functional fragment or variant thereof, that is capable of binding TGFβ, e.g., an extracellular domain of TGFβ receptor type I or an extracellular domain of TGFβ receptor type II.

In some embodiments, the TGFβ antagonist comprises any amino acid sequence of Table 6, or an amino acid sequence substantially identical thereto (e.g., 75%, 8-0%, 85%, 90%, 95%, or 99.9% identical thereto), or having at least one amino acid alteration, but not more than five, ten, fifteen, or twenty alterations (e.g., substitutions, deletions, or insertions, e.g., conservative substitutions).

In some embodiments, the immune cell engager comprises an NK cell engager that mediates binding to and activation of, an NK cell. In other embodiments, the immune cell engager comprises an NK cell engager that mediates binding to but not activation of, an NK cell. Exemplary NK cell engagers can be chosen from an antibody molecule, e.g., an antigen binding domain, or ligand that binds to (e.g., activates NKp30, NKp40, NKp44, NKp46, NKG2D, DNAM1, DAP10, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160. In some embodiments, the NK cell engager is an antibody molecule, e.g., an antigen binding domain that binds to NKp30 or NKp46.

In some embodiments, the immune cell engager comprises a T cell engager that mediates binding to and activation of, a T cell. In some embodiments, the immune cell engager comprises a T cell engager that mediates binding to but not activation of, a T cell.

In other embodiments of the multispecific molecule, the NK cell engager is a ligand, optionally, the ligand further comprises an immunoglobulin constant region, e.g., an Fc region. For example, the ligand of NKp44 or NKp46 is a viral HA; the ligand of DAP10 is a co-receptor for NKG2D; the ligand of CD16 is a CD16a/b ligand, e.g., a CD16a/b ligand further comprising an antibody Fc region.

In other embodiments, the immune cell engager mediates binding to, or activation of, or both of, one or more of a B cell, T cell, a macrophage, or a dendritic cell.

In other embodiments of the multispecific molecule, the T cell engager is an agonist of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager binds to, but does not activate CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226.

In some embodiments, the immune cell engager comprises a B cell, macrophage, and/or dendritic cell engager chosen from one or more of: CD40 ligand (CD40L) or a CD70 ligand; an antibody molecule that binds to CD40 or CD70; an antibody molecule to OX40; an OX40 ligand (OX40L); an agonist of a Toll-like receptor (e.g., a TLR4, e.g., a constitutively active TLR4 (caTLR4) or a TLR9 agonist); a 41BB; a CD2 agonist; a CD47; or a STING agonist, or a combination thereof.

In some embodiments, the B cell engager is a CD40L, an OX40L, or a CD70 ligand, or an antibody molecule that binds to OX40, CD40 or CD70.

In other embodiments, the macrophage cell engager is a CD2 agonist; a CD40L; an OX40L; an antibody molecule that binds to OX40, CD40 or CD70; an agonist of a Toll-like receptor (TLR)(e.g., a TLR4, e.g., a constitutively active TLR4 (caTLR4) or a TLR9 agonist); CD47; or a STING agonist.

In yet other embodiments, the dendritic cell engager is a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist. For example, the STING agonist can include a cyclic dinucleotide, e.g., a cyclic di-GMP (cdGMP), a cyclic di-AMP (cdAMP), or a combination thereof, optionally with 2′,5′ or 3′,5′ phosphate linkages. The STING agonist can be covalently coupled to the multispecific or multifunctional MPL antagonist molecule, e.g., by known techniques.

In other embodiments, the multispecific molecule can include a cytokine molecule chosen from: interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine can be a monomer or a dimer. For example, the cytokine molecule can further include a receptor dimerizing domain, e.g., an IL15Ralpha dimerizing domain. In other embodiments, the cytokine molecule (e.g., IL-15) and the receptor dimerizing domain (e.g., an IL15Ralpha dimerizing domain) are not covalently linked, e.g., are non-covalently associated.

In some embodiments of any of the multispecific molecules disclosed herein, the second tumor-targeting moiety, e.g., the tumor targeting moiety which targets a target other than MPL, is chosen from an antibody molecule to a cancer antigen chosen from mesothelin, PDL1, HER3, IGF1R, FAP, CD47 or CD123. For example, the tumor-targeting moiety can include an antibody molecule (e.g., Fab or scFv) that binds to mesothelin or PDL1. In some embodiments, the tumor-targeting moiety binds to PDL1 and inhibits an interaction of PDL1 with PD1. In other embodiments, the tumor-targeting moiety binds to PDL1 and does not inhibit an interaction of PD L1 with PD1.

In some embodiments, the multispecific molecule comprises three or four binding specificities or functions, e.g., is a trispecific or a tetraspecific molecule. In some embodiments, the multispecific or multifunctional molecule comprises (i) at least two tumor-targeting moieties, the immune cell engager, and the cytokine molecule; (ii) the tumor-targeting moiety, the immune cell engager, and the stromal modifying moiety; or (iii) at least two tumor-targeting moieties that bind to two cancer antigens chosen from MPL, mesothelin, PDL1, HER3, Fibroblast Activation Protein (FAP), or insulin growth factor 1R (IGF1R), CD47 or CD123, provided that the two cancer antigens are not FAP and IGF1R; and a cytokine molecule.

In some embodiments, the immunoglobulin constant region (e.g., an Fc region) is linked, e.g., covalently linked to, one or more of the MPL-targeting moieties, the immune cell engager, or the cytokine molecule.

In some embodiments, the multispecific molecule further comprises a linker, e.g., a linker between one or more of: the MPL-targeting moiety and the cytokine molecule, the MPL-targeting moiety and the immune cell engager, the cytokine molecule or and the immunoglobulin chain constant region (e.g., the Fc region), the targeting moiety and the immunoglobulin chain constant region, or the immune cell engager and the immunoglobulin chain constant region.

In some embodiments, the linker is selected from: a cleavable linker, a non-cleavable linker, a peptide linker, a flexible linker, a rigid linker, a helical linker, or a non-helical linker. In some embodiments, the linker is a peptide linker. In some embodiments, the peptide linker comprises Gly and Ser.

In some embodiments, the multispecific or multifunctional polypeptide is a bispecific molecule comprising a first and a second non-contiguous polypeptides, wherein:

(i) the first polypeptide includes, e.g., in the N- to C-orientation, a tumor-targeting moiety, e.g., an antibody molecule (e.g., a first portion of a first antigen domain, e.g., a first VH-CH1 of a Fab molecule), that binds to, e.g., a cancer antigen, e.g., a solid tumor, a stromal or a hematological antigen, connected, optionally via a linker to, a cytokine molecule, a stromal modifying moiety, or an immune cell engager, e.g., an antibody molecule, e.g., a scFv that binds to an immune cell antigen; and

(ii) the second polypeptide includes, e.g., in the N- to C-orientation, a second portion of the first antigen domain, e.g., a first VL-CL of the Fab, that binds to, e.g., a cancer antigen, e.g., a solid tumor, a stromal or a hematological antigen (e.g., the same tumor or stromal antigen bound by the first VH-CH1).

In some embodiments, the multispecific or multifunctional polypeptide is a bispecific molecule comprising a first and a second non-contiguous polypeptides, wherein:

(i) the first polypeptide includes, e.g., in the N- to C-orientation, a tumor-targeting moiety, e.g., an antibody molecule (e.g., a first portion of a first antigen domain, e.g., a first VH-CH1 of a Fab molecule), that binds to, e.g., a cancer antigen, e.g., a solid tumor, a stromal or a hematological antigen, connected, optionally, via a linker to, a first domain that promotes association between the first and the second polypeptide (e.g., a first immunoglobulin constant domain (e.g., a first Fc molecule as described herein);

(ii) the second polypeptide includes, e.g., in the N- to C-orientation, a cytokine molecule, a stromal modifying moiety, or an immune cell engager (e.g., an antibody molecule, e.g., a scFv, that binds to an immune cell antigen), connected, optionally, via a linker to, a second domain that promotes association between the first and the second polypeptide (e.g., a second immunoglobulin constant domain (e.g., a second Fc molecule as described herein); and (iii) the third polypeptide includes, e.g., in the N- to C-orientation, a second portion of the first antigen domain, e.g., a first VL-CL of the Fab, that binds to the cancer antigen.

In some embodiments, the multispecific molecule comprises:
a) a first polypeptide comprising a domain that promotes association of the first and second polypeptide, e.g., an Fc molecule; and a polypeptide selected from: an MPL-targeting moiety or an immune cell engager;
b) a second polypeptide selected from an MPL-targeting moiety or an immune cell engager;
c) a third polypeptide comprising a domain that promotes association of the first and third polypeptide, e.g., an Fc molecule; and a polypeptide selected from: an MPL-targeting moiety, an immune cell engager or a cytokine molecule; and
d) optionally, a fourth polypeptide selected from an MPL-targeting moiety, or an immune cell engager.
   wherein the multispecific or multifunctional MPL antagonist molecule comprises one MPL-targeting moieties and an immune cell engager or a cytokine molecule; or two MPL-targeting moieties.

In some embodiments, the multispecific molecule comprises:

an MPL-targeting moiety and an immune cell engager;

an MPL-targeting moiety and a cytokine molecule; or

two MPL-targeting moieties;

further comprising a dimerization domain.

In some embodiments, the multispecific molecule comprises one MPL targeting moiety and an immune cell engager, wherein the MPL-targeting moiety is an antibody molecule that binds MPL and the immune cell engager binds NKp46 or NKp30.

In some embodiments, the multispecific molecule comprises one MPL targeting moiety and a cytokine molecule, wherein the MPL-targeting moiety is an antibody molecule that binds MPL and the cytokine is IL2.

In some embodiments, the multispecific molecule comprises two MPL-targeting moieties wherein the MPL-targeting moieties are two antibody molecules with non-overlapping antigen binding sites, e.g., antibody molecules that bind to non-overlapping epitopes on the MPL protein.

In some embodiments, the multispecific molecule comprises the following non-contiguous polypeptides:

i) a first polypeptide which comprises, e.g., in the N- to C-orientation, an MPL-targeting moiety, e.g., an antibody molecule (e.g., a first portion of a first antigen domain, e.g., a first VH-CH1 of a Fab molecule) that binds to, e.g., an MPL antigen; a domain that promotes association of the first and third polypeptide, e.g., an Fc molecule;

ii) a second polypeptide which comprises, e.g., in the N- to C-orientation, an MPL-targeting moiety, e.g., an antibody molecule (e.g., a second portion of a first antigen domain, e.g., a first VL-CL of a Fab molecule) that binds to, e.g., an MPL antigen (e.g., the same antigen bound by the first VH-CH1); and

iii) a third polypeptide which comprises, e.g., in the N- to C-orientation, a domain that promotes association of the first and third polypeptides, e.g., an Fc molecule and a cytokine molecule.

In some embodiments, the multispecific molecule comprises the following non-contiguous polypeptides:

i) a first polypeptide which comprises, e.g., in the N- to C-orientation, an MPL-targeting moiety, e.g., an antibody molecule (e.g., a first portion of a first antigen domain, e.g., a first VH-CH1 of a Fab molecule) that binds to, e.g., an MPL antigen; a domain that promotes association of the first and third polypeptide, e.g., an Fc molecule;

ii) a second polypeptide which comprises, e.g., in the N- to C-orientation, an MPL-targeting moiety, e.g., an antibody molecule (e.g., a second portion of a first antigen domain, e.g., a first VL-CL of a Fab molecule) that binds to, e.g., an MPL antigen (e.g., the same antigen bound by the first VH-CH1);

iii) a third polypeptide which comprises, e.g., in the N- to C-orientation, an immune cell engager, e.g., an antibody molecule (e.g., a first portion of a second antigen domain, e.g., a first VH-CH1 of a Fab molecule) that binds to, e.g., an immune cell engager; a domain that promotes association of the first and third polypeptide, e.g., an Fc molecule; and

iv) a fourth polypeptide which comprises, e.g., in the N- to C-orientation, an immune cell engager, e.g., an antibody molecule (e.g., a second portion of a second antigen domain, e.g., a first VL-CL of a Fab molecule) that binds to, e.g., an immune cell engager (e.g., the same antigen bound by the second VH-CH1).

### Nucleic Acids, Host Cells, Vectors and Methods

In another aspect, the disclosure provides an isolated nucleic acid molecule encoding any multispecific molecule described herein.

In another aspect, the disclosure provides an isolated nucleic acid molecule, which comprises the nucleotide sequence encoding any of the multispecific molecules described herein, or a nucleotide sequence substantially homologous thereto (e.g., at least 95% identical thereto).

In another aspect, the disclosure provides a vector, e.g., an expression vector, comprising one or more of any nucleic acid molecules described herein.

In another aspect, the disclosure provides a host cell comprising a nucleic acid molecule or a vector described herein.

In another aspect, the disclosure provides a method of making, e.g., producing, a multispecific molecule described herein, comprising culturing a host cell described herein, under suitable conditions, e.g., conditions suitable for gene expression and/or homo- or heterodimerization.

In another aspect, the disclosure provides a pharmaceutical composition comprising a multispecific molecule described herein and a pharmaceutically acceptable carrier, excipient, or stabilizer.

In another aspect, the disclosure provides a method of treating a cancer, comprising administering to a subject in need thereof a multispecific or multifunctional molecule described herein, wherein the multispecific antibody is administered in an amount effective to treat the cancer, e.g., a hematological cancer, e.g., myelofibrosis.

Exemplary cancers that can be treated using the multispecific molecules described herein include, but are not limited to the tumor, e.g., a hematological cancer, including, but not limited to, a B-cell or T cell malignancy, e.g., Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), myelofibrosis, acute myeloid leukemia (AML), chronic myeloid leukemia (CML), myelodysplastic syndrome, multiple myeloma, and acute lymphocytic leukemia (ALL). In one embodiment, the cancer is myelofibrosis.

In other embodiments, the cancer is a solid tumor cancer, or a metastatic lesion. In some embodiments, the solid tumor cancer is one or more of pancreatic (e.g., pancreatic adenocarcinoma), breast, colorectal, lung (e.g., small or non-small cell lung cancer), skin, ovarian, or liver cancer. In some embodiments, the cancer is a hematological cancer.

In some embodiments, the method further comprises administering a second therapeutic treatment. In some embodiments, second therapeutic treatment comprises a therapeutic agent (e.g., a chemotherapeutic agent, a biologic agent, hormonal therapy), radiation, or surgery. In some embodiments, therapeutic agent is selected from: a chemotherapeutic agent, or a biologic agent.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1D depicts a schematic representation of the MPL receptor, also known as the TPO receptor, in its inactive form (FIG. 1A), or bound by a ligand or antibody fragments (FIGS. 1B-1D). Exemplary MPL-binding molecules described herein are depicted in FIGS. 1C-1D.

### DETAILED DESCRIPTION OF THE INVENTION

Disclosed herein are multispecific molecules (also referred to herein as “multifunctional molecules”) that include a one or more binding specificities (or functionalities), wherein the molecules comprise a binding specificity that selectively localizes to an MPL-expressing cell, e.g., a cancer cell (e.g., it includes an MPL-targeting moiety). Without wishing to be bound by theory, the multispecific or multifunctional molecules described herein can bind to a single MPL protein and inhibit MPL dimerization, e.g., even in the presence of TPO, which in turn reduces an MPL biological activity. In embodiments, the multispecific or multifunctional molecules described herein antagonize MPL dimerization, e.g., TPO mediated MPL dimerization, by binding to one or more epitopes on the ECD of a single MPL polypeptide.

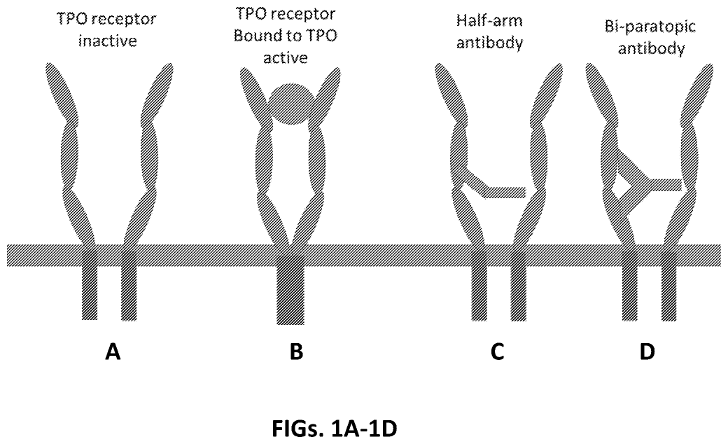

In some embodiments, the multispecific molecules or MPL-binding molecules disclosed herein include a single half-arm antibody that binds to only one epitope on one MPL protein ECD (see FIG. 1C). In other embodiments, the multispecific molecules or MPL-binding molecules disclosed herein include a bispecific, e.g., a biparatopic, antibody molecule that binds to two epitopes on the same MPL protein ECD (see FIG. 1D). Without wishing to be bound by theory, the multispecific molecules or MPL-binding molecules are expected to be able to block a conformation change required for subsequent activation even in the presence of the natural ligand TPO. The multispecific molecules or MPL-binding molecules disclosed herein can be designed by converting a bivalent anti-MPL antibody, e.g., a bivalent agonistic antibody, into either a monovalent (half-arm) antibody molecule, or by combining two different antibodies, e.g., agonistic antibodies, that bind to two different epitopes on MPL and converting them into a single bispecific, e.g., biparatopic, antagonistic antibody molecule.

In some embodiments, the multispecific molecule or MPL-binding molecule shown in FIGS. 1C and 1D can further comprise one or more of an immune cell engager, a cytokine molecule, or a tumor targeting molecule (e.g., a tumor targeting molecule that targets a tumor target other than MPL). In some embodiments, the multispecific molecule or MPL-binding molecule shown in FIGS. 1C and 1D further comprise one or more of an anti-CD41 antibody molecule, an anti-CD177 antibody molecule, an anti-PDL1 antibody molecule, an anti-CD3 antibody molecule, an anti-TGFβ antibody molecule, a TGFβtrap polypeptide (e.g., a polypeptide comprising a portion of TGFβ receptor that is capable of binding TGFβ), an anti-IL1β antibody molecule, an IL1β trap polypeptide (e.g., a polypeptide comprising a portion of IL1β receptor that is capable of binding IL1β), an anti-CXCL10 antibody molecule, an anti-MS4A3 antibody molecule, an anti-OLFM4 antibody molecule, an anti-CD66b antibody molecule, an anti-cKit antibody molecule, an anti-FLT3 antibody molecule, or an anti-CD133 antibody molecule.

Accordingly, provided herein are, inter alia, multispecific molecules (e.g., multispecific or multifunctional antibody molecules) that bind to one or more regions, e.g., one or more epitopes, on a single MPL protein (e.g., the same MPL protein). In one embodiment, the multispecific molecule is or comprises two MPL-targeting moieties, e.g., it is a biparatopic molecule, e.g., it binds to two different epitopes (e.g., non-overlapping epitopes) on the same MPL protein. In embodiments, the biparatopic molecule can further comprise one or more of an immune cell engager, a cytokine molecule, or a tumor targeting molecule (e.g., a second tumor targeting molecule that targets a tumor target other than MPL).

In another embodiment, the multispecific molecule is or comprises a single MPL-targeting moiety, e.g., a half-arm antibody against MPL (e.g., a Fab fused to an immunoglobulin constant domain (e.g., a first Fc constant region (e.g., a first CH2-CH3)). Optionally, the half-arm antibody is dimerized (e.g., homo- or heterodimerized) to a second immunoglobulin constant domain, e.g., a second heavy chain constant region, e.g., a second Fc constant region). In embodiments, the MPL targeting moiety and/or the second immunoglobulin constant domain can further comprise one or more of an immune cell engager, a cytokine molecule, or a tumor targeting molecule (e.g., a second tumor targeting molecule that targets a tumor target other than MPL).

A number of different point mutations, deletions or insertions of JAK2 have been linked to hematologic diseases. See, Haan C, et al., J Cell Mol Med. 2010; 14(3):504-527, incorporated by reference herein in its entirety. Mutations in the JH2 domain of JAK2 are concentrated in three regions encoded by exon 14, exon 16, and exon 12. Disease-associated mutations in the JH1 domain of JAK2 have also been identified. One mutation, V617F (a change of valine to phenylalanine at position 617), renders hematopoietic cells more sensitive to cytokines such as TPO. It is hypothesized that in addition to destabilize the JH2-JH1 autoinhibitory interaction, V617F may also promote JH2-mediated positive interactions that are important for signaling. See, Silvennoinen and Hubbard, Blood. 2015 May 28; 125(22): 3388-3392, incorporated by reference herein in its entirety. Without wishing to be bound by theory, mutations in JAK2, such as the V617F mutation, may cause a conformational change in cytokine receptors such as MPL; and such a conformational change may be recognized by a MPL-binding molecule or a multispecific molecule disclosed herein. In one embodiment, the MPL-binding molecule or multispecific molecule disclosed herein binds preferentially to an MPL expressed on a cancer cell over an MPL expressed on a non-cancerous cell (e.g., binds to an MPL expressed on a cancer cell with a greater affinity, e.g., at least 2, 5, 10, 20, 30, 40, 50, 75 or 100 times greater affinity, than when the MPL-binding molecule or multispecific molecule binds to an MPL expressed on a non-cancerous cell). In one embodiment, the MPL-binding molecule or multispecific molecule disclosed herein binds preferentially to an MPL associated with a mutated JAK2 (e.g., a JAK2 comprising a V617F mutation) over an MPL associated with a wild-type JAK2 (e.g., binds to an MPL associated with a mutated JAK2 (e.g., a JAK2 comprising a V617F mutation) with a greater affinity, e.g., at least 2, 5, 10, 20, 30, 40, 50, 75 or 100 times greater affinity, than when the multispecific molecule or MPL-binding molecule binds to an MPL associated with a wild-type JAK2). In one embodiment, the MPL-binding molecule or multispecific molecule disclosed herein binds preferentially to an MPL expressed on a cell expressing a mutated JAK2 (e.g., a JAK2 comprising a V617F mutation) over an MPL expressed on a cell expressing a wild-type JAK2 (e.g., binds to an MPL expressed on a cell expressing a mutated JAK2 (e.g., a JAK2 comprising a V617F mutation) with a greater affinity, e.g., at least 2, 5, 10, 20, 30, 40, 50, 75 or 100 times greater affinity, than when the multispecific molecule or MPL-binding molecule binds to an MPL expressed on a cell expressing a wild-type JAK2). In one embodiment, the MPL-binding molecule or multispecific molecule disclosed herein binds to an epitope that is only present in MPL when MPL is associated with a mutated JAK2 (e.g., a JAK2 comprising a V617F mutation), but not when MPL is associated with a wild-type JAK2.

Additionally disclosed are nucleic acids encoding the aforesaid multispecific molecules, methods of producing the aforesaid molecules, and methods of treating a cancer using the aforesaid molecules.

### Definitions

Certain terms are defined below.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

As used herein, the term “molecule” as used in, e.g., antibody molecule, cytokine molecule, receptor molecule, includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., naturally-occurring) molecule remains.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)2, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

The term “biparatopic antibody” as used herein, refers to an antibody molecule that binds to two different epitopes on the same target receptor, e.g., an antibody that binds to two different epitopes on the MPL receptor.

The term “non-overlapping binding sites,” e.g., “non-overlapping epitopes,” include binding sites, e.g., epitopes (e.g., linear or conformational epitopes) that are different from each other. In some embodiments, the non-overlapping binding sites, e.g., epitopes, are bound by two different binding agents, e.g., antibody molecules, that do not compete for binding with each other. In other embodiments, the non-overlapping binding sites, e.g., epitopes, are bound by two different antibody molecules that partially overlap, e.g., compete for binding with each other.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a molecule that can provoke an immune response, e.g., involving activation of certain immune cells and/or antibody generation. Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site,” or “binding portion” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

“Cancer” as used herein can encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

“Immune effector cell,” as that term is used herein, refers to a cell that is involved in an immune response, e.g., in the promotion of an immune effector response. Examples of immune effector cells include, but are not limited to, T cells, e.g., alpha/beta T cells and gamma/delta T cells, B cells, natural killer (NK) cells, natural killer T (NK T) cells, and mast cells.

The term “effector function” or “effector response” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

### MPL-Targeting Moieties

The present disclosure provides, inter alia, multispecific molecules (e.g., bispecifics, e.g., biparatopic molecules), that include, e.g., are engineered to contain, one or more MPL specific targeting moieties that direct the molecule to a cell, e.g., a tumor or hematopoietic cell.

“MPL,” refers to myeloproliferative leukemia protein (MPL), which is also known as CD110 or TPOR (Thrombopoieitin receptor), and relates to human MPL protein and species, isoforms, and other sequence variants thereof. Thus, MPL can be the native, full-length protein or can be a truncated fragment or a sequence variant (e.g., a naturally occurring isoform, or recombinant variant) that retains at least one biological activity of the native protein, e.g., platelet production. MPL protein has two extracellular cytokine receptor domains and two intracellular cytokine receptor box motifs. MPL is primarily expressed on human hematopoietic cells and regulates, e.g., megakaryopoiesis (reviewed in Ng et al., “Mpl expression on megakaryocytes and platelets is dispensable for thrombopoiesis but essential to prevent myeloproliferation”, PNAS, Vol. 111, Issue 16, 5884-5889, doi: 10.1073/pnas.1404354111).

Binding of thrombopoietin (TPO), the natural ligand of MPL, to the extracellular domain of MPL induces MPL dimerization which leads to a conformational change resulting in intracellular phosphorylation and activation of the JAK2 kinase pathway. MPL biological activity can be modulated with anti-MPL antibodies, e.g., antibodies described in U.S. Pat. Nos. 7,993,642, 6,342,220, 8,034,903, and US 2012/0269814A1, the entire contents of which are incorporated herein by reference.

In certain embodiments, the multispecific molecules disclosed herein include an MPL-targeting moiety. The MPL targeting moiety can be chosen from an antibody molecule (e.g., an antigen binding domain as described herein), a receptor or a receptor fragment, or a ligand or a ligand fragment, or a combination thereof. In some embodiments, the MPL targeting moiety associates with, e.g., binds to, a cancer or hematopoietic cell (e.g., a molecule, e.g., antigen, present on the surface of the cancer or hematopoietic cell). In certain embodiments, the MPL moiety targets, e.g., directs the multispecific molecules disclosed herein to a cancer or hematopoietic cell. In some embodiments, the cancer is a hematological cancer, e.g., myelofibrosis.

In some embodiments, the multispecific molecule, e.g., the MPL-targeting moiety, binds to an MPL antigen on the surface of a cell, e.g., a cancer or hematopoietic cell. The MPL antigen can be present on a primary tumor cell, or a metastatic lesion thereof. In some embodiments, the cancer is a hematological cancer, e.g., myelofibrosis. For example, the MPL antigen can be present on a tumor, e.g., a tumor of a class typified by having one or more of: limited tumor perfusion, compressed blood vessels, or fibrotic tumor interstitium.

Exemplary MPL Targeting Moieties

The multispecific molecules described herein can include an MPL targeting moiety that comprises an anti-MPL antibody or antigen-binding fragment thereof described in U.S. Pat. Nos. 7,993,642, 6,342,220, 8,034,903, and US 2012/0269814A1, the entire contents of which are incorporated herein by reference.

In one embodiment, the MPL-targeting moiety includes an antibody molecule (e.g., Fab or scFv) that binds to MPL. In some embodiments, the antibody molecule to MPL comprises one, two, or three CDRs from any of the heavy chain variable domain sequences of Table 1, or a closely related CDR, e.g., CDRs which have at least one amino acid alteration, but not more than two, three or four alterations (e.g., substitutions, deletions, or insertions, e.g., conservative substitutions) from any the CDR sequences of Table 1. In some embodiments, the antibody molecule to MPL comprises a heavy chain variable domain sequence chosen from any of the amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

Alternatively, or in combination with the heavy chain to MPL disclosed herein, the antibody molecule to MPL comprises one, two, or three CDRs from any of the light chain variable domain sequences of Table 1, or a closely related CDR, e.g., CDRs which have at least one amino acid alteration, but not more than two, three or four alterations (e.g., substitutions, deletions, or insertions, e.g., conservative substitutions) from any of the CDR sequence of Table 1. In some embodiments, the antibody molecule to MPL comprises a light chain variable domain sequence chosen from any of the amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

In one embodiment, the MPL-targeting moiety includes an antibody molecule (e.g., Fab or scFv) that binds to MPL. In some embodiments, the antibody molecule to MPL comprises one, two, or three CDRs from the heavy chain variable domain sequence of SEQ ID NO: 1 (see Table 1), or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO: 1.

In embodiments, the antibody molecule to MPL includes the heavy chain variable domain sequence of SEQ ID NO: 1), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 1.

In embodiments, the antibody molecule to MPL is a Fab and further comprises a heavy chain constant region (CH1) having the amino acid sequence of SEQ ID NO:69 (see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 69.

Alternatively, or in combination with the heavy chain to MPL disclosed herein, the antibody molecule to MPL comprises one, two, or three CDRs from the light chain variable domain sequence of SEQ ID NO: 2 (see Table 1), or a closely related CDR, e.g., CDRs which have at least one amino acid alteration, but not more than two, three or four alterations (e.g., substitutions, deletions, or insertions, e.g., conservative substitutions) from the CDR sequence of SEQ ID NO: 2.

In some embodiments, the antibody molecule to MPL comprises the light chain variable domain sequence of SEQ ID NO: 2, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 2.

In some embodiments, the antibody molecule to MPL is a Fab and further comprises a light chain constant region (CL(kappa)) having the amino acid sequence SEQ ID NO: 70 (see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 70.

In some embodiments, the antibody molecule to MPL is a Fab and further comprises a light chain constant region (CL(lambda)) having the amino acid sequence SEQ ID NO: 71(see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 71.

In other embodiments, the antibody molecule to MPL comprises one, two, or three CDRs from the heavy chain variable domain, and one, two, or three CDRs from the light chain variable domain sequence of any of the pairs of variable domains presented in Table 1, e.g., variable heavy and variable light domains from the same antibody molecule, e.g., variable heavy domain of SEQ ID NO: 3 and variable light domain of SEQ ID NO:4.

In embodiments, the antibody molecule to MPL includes the heavy chain variable domain sequence of SEQ ID NO: 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 3, and the light chain variable domain sequence of SEQ ID NO: 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 4.

In embodiments, the antibody molecule to MPL is a single chain Fv comprising the sequence of SEQ ID NO: 49, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 49.

In embodiments, the antibody molecule further comprises:

(i) a heavy chain constant region (CH1) having the amino acid sequence of SEQ ID NO: 69 (see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 69;

(ii) a light chain constant region (CL(kappa)) having the amino acid sequence SEQ ID NO: 70 (see Table 2); or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 70; or

(iii) a light chain constant region (CL(lambda)) having the amino acid sequence SEQ ID NO: 71(see Table 2), or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequence of SEQ ID NO: 71.

In embodiments, the antibody molecule to MPL is a single chain Fv comprising any of SEQ ID NOs: 50-56, or amino acid sequences substantially identical thereto (e.g., 95% to 99.9% identical thereto), or having at least one amino acid alteration, but not more than five, ten or fifteen alterations (e.g., substitutions, deletions, or insertions, e.g., conservative substitutions) to the amino acid sequences of SEQ ID NOs: 50-56.

TABLE 1
Amino acid sequences of variable regions
of exemplary anti-MPL antibodies.
U.S. Pat. No. 7,993,642

| | | |
|---|---|---|
| SEQ ID | VA7 | QVQLQQSGPELVKPGASVKISCKASG |
| NO: 1 | VH | YAFSSSWMNWVKQRPGKGLEWIGRTY |
| | | PGDGDTNYNGKFKGKATLTADKSSST |
| | | AYMQLSSLTSEDSAVYFCARGWILAD |
| | | GGYSFAYWGQGTLVTVSA |
| SEQ ID | VA7 | DIVMTQAAPSIPVTPGESVSISCRSS |
| NO: 2 | VL | KSLLHSNGNTYLYWFLQRPGQSPQLL |
| | | IYRMSNLASGVPDRFSGSGSGTAFTL |
| | | RISRVEAEDVGIYYCMQHLEYPPTFG |
| | | TGTKLEIK |
| SEQ ID | VA130- | QVQLQQSGPELVKPGASVKISCKASG |
| NO: 3 | VH | YAFSSSWMNWVKQRPGKGLEWIGRIY |
| | | PGDGDTNYNGKFKGKATLTADKSSST |
| | | AYIQLSSLTSEDSAVYFCARGYADYS |
| | | FAYWGQGTLVTVSA |
| SEQ ID | VA130 | DIVMTQAAPSVPVTPGESVSISCRSS |
| NO: 4 | VL | KSLLHSNGNTYLYWFLQRPGQSPQLL |
| | | IYRMSNLASGVPDRFSGSGSGTAFTL |
| | | RISRVEAEDVGVYYCMQHLEYPYTFG |
| | | SGTKLEIK |
| SEQ ID | VA259 | QVQLQQSGPELVKPGASVKISCKASG |
| NO: 5 | VH | YAFSSSWMNWVKQRPGKGLEWIGRIY |
| | | PGDGETNYNGKFKGKATLTADKSSNT |
| | | AYMQLSSLTSEDSAVYFCARGFGDYS |
| | | FAYWGQGTLVTVSA |
| SEQ ID | VA259 | DIVMTQAAPSVPVTPGESVSISCRSS |
| NO: 6 | VL | KSLLHSNGNTYLYWFLQRPGQSPQLL |
| | | IYRVISNLASGAPDRFSGSGSGTAFT |
| | | LRISRVETEDVGVYYCMQHLEYPYTF |
| | | GSGTKLEIK |
| SEQ ID | VBI7B | QVQLQQSGPELVKPGASVKISCKASG |
| NO: 7 | VH | YTFSSSWMNWVKQRPGKGLEWIGRIY |
| | | PGDGDTNYNGKFKGKATLTADKSSST |
| | | AYMQLSSLTSEDSAVYFCASGYADYS |
| | | FAYWGQGTLVTVSA |
| SEQ ID | VB17B | DIVMTQAAPSVPVTPGESVSISCRSS |
| NO: 8 | VL | KSLLHSNGNTYLYWFLQRPGQSPQLL |
| | | IYRMSNLASGVPDRFSGSGSGTAFTL |
| | | RISRVEAEDVGVYYCMQHLEYPYTFG |
| | | SGTKLEIK |
| SEQ ID | VB12B | QVQLQQSGPELVKPGASVKISCKASG |
| NO: 9 | VH | YAFSRSWMNWVKQRPGKGLEWIGRIY |
| | | PGDGDTNYNGKFKGKATLTADKSSST |
| | | AYMQLSSLTSEDSAVYFCASGYDDYS |
| | | FAYWGQGTLVTVSA |
| SEQ ID | VB12B | DIVMTQAAPSVPVTPGESVSISCRSS |
| NO: 10 | VL | KSLLHSNGNTYLYWFLQRPGQSPQLL |
| | | IYRMSNLASGVPDRFSGSGSGTAFTL |
| | | RISRVEAEDVGVYYCMQHLEYPYTFG |
| | | SGTKLEIK |
| SEQ ID | VB140 | QVQLQQSGPELVKPGASVKISCRAFG |
| NO : 11 | VH | YAFSNSWMNWVKQRPGKGLEWIGRIY |
| | | PGDGETNNNGKFKGKATLTADKSSST |
| | | AYMQLSSLTSEDSAVYFCARGYGDYS |
| | | FAYWGQGTLVTVSA |
| SEQ ID | VB140 | DIVMTQAAPSVPVTPGESVSISCRSS |
| NO: 12 | VL | KSLLHSNGNTYLYWFLQRPGQSPQLL |
| | | IYRMSNLASGVPDRFSGSGSGAAFTL |
| | | RISRVEAEDVGVYYCMQHLEYPYTFG |
| | | SGTKLEIK |
| SEQ ID | VB33 | QVQLQQPGAELVKPGASVKLSCKASG |
| NO: 13 | VH | YTFTNYWVNWVKQRPGRGLEWIGRIH |
| | | PSDSETHCNQKFKRKATLTVNKSSST |
| | | AYIQLHSLTSEDSAVYYCTSGGWFAY |
| | | WGQGTLVTVSA |
| SEQ ID | VB33 | DIVVITQAAPSVPVTPGESVSISCRS |
| NO: 14 | VL | SKSLLYSNGNIYLYWFLQRPGQSPQL |
| | | LIYRMSNLASGVPDRFSGSGSGTAFT |
| | | LRISRVEAEDVGVYYCMQHLEYPYTF |
| | | GSGTKLEIK |
| SEQ ID | VB45B: | QVQLQQSGPELVKPGASVKISCKASG |
| NO: 15 | VH | YAFSSSWMNWVKQRPGKGLEWIGRIY |
| | | PGDGETNNNGKFKGKATLTADKSSTT |
| | | AYMQLSSLTSEDSAVYFCARGYGDYS |
| | | FAYWGQGTLVTVSA |
| SEQ ID | VB45B: | DIVMTQAAPSVPVTPGESVSISCRSS |
| NO: 16 | VL | KSLLHSNGNTYLYWFLQRPGQSPQLL |
| | | IYRMSNLASGVPDRFSGSGSGAAFTL |
| | | RISRVEAEDVGVYYCMQHLEYPYTFG |
| | | SGTKLEIK |
| SEQ ID | VB8B: | QVQLQQSGPELVKPGASVKISCKASG |
| NO: 17 | VH | YAFSTSWMNWVKQRPGKGLEWIGRIY |
| | | PGDGEANYNGKFKGKATLTADKSSSS |
| | | AYMQLSSLTSEDSAVYFCARGYGDYS |
| | | FAYWGQGTLVTVSA |
| SEQ ID | VB8B: | DIVMTQAAPSVPVTPGESVSISCRSS |
| NO: 18 | VL | KSLLHSNGNTYLYWFVIQRPGQSPQL |
| | | LIYRMSNLASGVPDRFSGSGSGTAFT |
| | | LRISRVEAEDVGVYYCMQHVEYPYTF |
| | | GSGTKLEIK |
| SEQ ID | VB115: | QVQLQQSGPELVKPGASVKISCKASG |
| NO: 19 | VH | YAFSSSWMNWVKQRPGKGPEWIGRIY |
| | | PGDGETNYNGKFKGKATLTADKSSST |
| | | VYMQLSSLTSEDSAVYFCARGYGDYS |
| | | FAYWGQGTLVTVSA |
| SEQ ID | VB115: | DIVMTQAAPSVPVTPGESVSISCRSS |
| NO: 20 | VL | KSLLHSNGNTYLYWFLQRPGQSPQLL |
| | | IYRMSNLASGVPDRFSGSGSGTAFTL |
| | | RISRVEAEDVGVYYCMQHLEYPYTFG |
| | | SGTKLEIK |
| SEQ ID | VB14B: | QVQLQQSGPELLNPGASVKISCKASG |
| NO: 21 | VH | YAFSRSWMNWVKQRPGKGLEWIGRIY |
| | | PGDGETNYNGKFKGKATLTADKSSTT |
| | | AYMQFSSLTSEDSAVYFCARGDGDYS |
| | | FAYWGQGTLVTVSA |
| SEQ ID | VB14B: | DIVMTQAAPSVPVTPGESVSISCRSS |
| NO: 22 | VL | KSLLHSNGNTYLYWFLQRPGQSPQLL |
| | | IYRMSNLASGVPDRFSGSGSGTAFTL |
| | | RISRVEAEDVGVYYCMQHLEYPYTFG |
| | | SGTKLEIK |
| SEQ ID | VB22B: | QVQLQQSGPELVKPGASVKISCKASG |
| NO: 23 | VH | YAFTNSWVINWVKQRPGKGLEWIGRI |
| | | YPGDGETIYNGKFRVKATLTADKSSS |
| | | TAYMDISSLTSEDSAVYFCARGYDDY |
| | | SFAYWGQGTLVTVSA |
| SEQ ID | VB22B: | DIVMTQAAPSIPVTPGESVSISCRSS |
| NO: 24 | VL | KSLLHSNGNTYLYWFLQRPGQSPQLL |
| | | IYRMSNLASGVPDRFSGSGSGTAFTL |
| | | RISRVEAEDVGVYYCMQHIEYPFTFG |
| | | SGTKLEIK |
| SEQ ID | VB16: | QVQLQQPGTELVRPGASVKLSCKASG |
| NO: 25 | VH | YTFTDYWVNWVKQRPGRGLEWIGRIH |
| | | PYDSETHYNQKFKNKATLTVDKSSST |
| | | AYIQLSSLTSEDSAVYYCASGGWFAS |
| | | WGQGTLVTVSA |
| SEQ ID | VB16: | DIVMTQAAPSVPVTPGESVSISCRSS |
| NO: 26 | VL | KSLLYSNGNTYLYWTLQRPGQSPQLL |
| | | IYRMSNLASGVPDRFSGSGSGTAFTL |
| | | TISSVEAEDVGVYYCMQHLEYPYTFG |
| | | SGTKLEIK |
| SEQ ID | VB157: | QVQLQQPGAELVKPGASVKLSCKASG |
| NO: 27 | VH | YTFTDYWMNWVKQRPGRGLEWIGRIH |
| | | PFDSETHCSQKFKNKATLTVDKSSNT |
| | | AYIQFSSLTSEDSAVYYCSSGGWFAY |
| | | WGQGTLVTVSA |
| SEQ ID | VB157: | DIVMTQAAPSVSVTPGESVSISCRSS |
| NO: 28 | VL | KSLLYSNGNIYLYWFLQRPGQSPQLL |
| | | IYRMSNLASGVPDRFSGSGSGTAFTL |
| | | KISRVEAEDVGVYYCMQHLEYPYTFG |
| | | SGTKLEIK |
| SEQ ID | VB4B: | QVQLQQSGPELVKPGASVKISCKASG |
| NO: 29 | VH | YAFTNSWMNWVRQRPGKGLEWIGRIY |
| | | PGDGETIYNGKFRVKATLTADKSSST |
| | | AYMEISSLTSEDSAVYFCARGYDDYS |
| | | FAYWGQGTLVTVSA |
| SEQ ID | VB4B: | DIVMTQAAPSVPVTPGESVSISCRSS |
| NO: 30 | VL | KSLLHNNGNTYLYWFLQRPGQSPQLL |
| | | IYRMSNLASGVPDRFSGSGSGTAFTL |
| | | RISRVEAEDVGVYYCMQHIEYPFTFG |
| | | SGTKLEIK |
| SEQ ID | VB51: | QVQLQQSGPELVKPGASVKISCKASG |
| NO: 31 | VH | YAFSNSWMNWVNQRPGKGLEWIGRIY |
| | | PGDGDTIYNGNFKGKATLTADKSSSI |
| | | AYMQLSSLTSEDSAVYFCTSGYDDYS |
| | | FAYWGQGTLVTVSA |
| SEQ ID | VB51: | DIVVITQAAPSLPVTPGESVSISCRS |
| NO: 32 | VL | SKSLLHSNGNTYLYWFLQRPGQSPQL |
| | | LIYRMSNLASGVPDRFSGSGSGTAFT |
| | | LRISRVEAEDVGVYYCMQHLEYPYTF |
| | | GSGTKLEIK |
| SEQ ID | AB317: | MVLASSTTSIHTMLLLLLMLAQPAMA |
| NO: 33 | VH | EVKLVESGGGLVKPGGSRKLSCAASG |
| | | FTFSSYTMSWVRQTPAKRLEWVATIS |
| | | SGSSTIYYADTVKGRFTISRDNAKNT |
| | | LFLQMTSLRSEDTAMYYCARRWFLDC |
| | | WGQGTTLTVSS |
| SEQ ID | AB317: | DIVLTQSPASLAVSLGQSVTISCRAS |
| NO: 34 | VL | ESVEYYGTSLVIQWYQQKPGQPPKLL |
| | | IYGASNVESGVPARFSGSGSGTDFSL |
| | | NIHPVEEDDIAMYFCQQSRKVPWTFG |
| | | GGTKLEIKDYKDDDDK |
| SEQ ID | AB324 | MVLASSTTSIHTMLLLLLMLAQPAMA |
| NO: 35 | VH | QVQLQQSGPELVKPGASVKISCKASG |
| | | YAFSSSWMNWVIKQRPGKGLEWIGRI |
| | | YPGDGDTNYNGKFKGKATLTADKSSS |
| | | TAYMQLSSLTSEDSAVYFCARARKTS |
| | | WFAYWGQGTLVTVSA |
| SEQ ID | AB324 | DIVLTQSQKFVISTSVGDRVSISCKA |
| NO: 36 | VL | SQNVGNIIAWYQQKPGQSPKALIYLA |
| | | SYRYSGVPDRFrGSGSGTDFTLTISN |
| | | VQSEDLAEYFCQQYSSSPLTFGAGTK |
| | | LEIKDYKDDDDK |
| SEQ ID | TA136: | DVQLQESGPGLVKPSQSLSLTCTVTG |
| NO: 37 | VH | YSITSDYAWSWIRQLPGNKLEWMGYI |
| | | TYSGYSIYNPSLKSRISISRDTSKNQ |
| | | LFLQLNSVTTEDTATYYCVGGYDNMD |
| | | YWGQGTSVTVSS |
| SEQ ID | TA136: | QIVLTQSPAIMSASPGEKVTLTCSAS |
| NO: 38 | VL | SSVSSSHLYWYQQKPGSSPKLWIYST |
| | | SNLASGVPARFSGSGSGTSYSLTISN |
| | | METEDAASYFCHQWSSYPWTFGGGTK |
| | | LEIK |
| SEQ ID | hVB22 | QVQLVQSGPEVKKPGASVKVSCKASGY |
| NO: 39 | B p-z: | TFTNSWMNWVRQRPGKGLEWMGRIYPG |
| | VH | DGETIYNGKFRVRVTITADESTSTAYM |
| | | ELSSLRSEDTAVYYCARGYDDYSFAYW |
| | | GQGTTVTVSS |
| SEQ ID | hVB22 | DIVMTQSALSLPVTPGEPASISCRSSK |
| NO: 40 | B p-z: | SLLHSNGNTYLYWFQQKPGQSPQLLIY |
| | VL | RMSNLASGVPDRESGSGSGTAETLKIS |
| | | RVEAEDVGVYYCMQHIEYPPTEGQGTK |
| | | LEIK |
| SEQ ID | hVB22 | QVQLVQSGPEVKKPGASVKVSCKASGY |
| NO: 41 | B g-e: | TFTNSWMNWVRQRPGKGLEWVGRIYPG |
| | VH | DGETIYNGKFRVRVTITADESTSTAYM |
| | | ELSSLRSEDTAVYYCARGYDDYSFAYW |
| | | GQGTTVTVSS |
| SEQ ID | hVB22 | DIVMTQSALSLPVTPGEPASISCRSSK |
| NO: 42 | B g-e: | SLLHSNGNTYLYWYLQKPGQSPQLLIY |
| | VL | RMSNLASGVPDRFSGSGSGTAFTLKIS |
| | | RVEAEDVGVYYCMQHIEYPFTFGQGTK |
| | | LEIK |
| SEQ ID | hVB22 | QVQLVQSGPEVKKPGASVKVSCKASGY |
| NO: 43 | B e: VH | TPTNSWMNWIRQRPGKGLEWIGRIYPG |
| | | DGETIYNGKIRVRVTITADESTSTAYM |
| | | ELSSLRSEDTAVYYCARGYDDYSFAYW |
| | | GQGTLVTVSS |
| SEQ ID | hVB22 | DIVMTQSALSLPVTPGEPASISCRSSK |
| NO: 44 | B e: VL | SLLHSNGNTYLYWYLQKPGQSPQLLIY |
| | | RMSNLASGVPDRFSGSGSGTAFTLKIS |
| | | RVEAEDVGVYYCMQHIEYPFTFGQGTK |
| | | LEIK |
| SEQ ID | hVB22 | QVQLVQSGPEVKKPGASVKVSCKASGY |
| NO: 45 | B u2- | TFTNSWMNWVRQRPGKGLEWIGRIYPG |
| | wz4: | DGETIYNGKFRVRVTITADESTSTAYM |
| | VH | QLSSLRSEDTAVYYCARGYDDYSFAYW |
| | | GQGTTVTVSS |
| SEQ ID | hVB22 | DIVMTQSPLSLPVTPGEPASISCRSSK |
| NO: 46 | B u2- | SLLHSNGNTYLYWFLQKPGQSPQLLIY |
| | wz4: | RMSNLASGVPDRFSGSGSGTDITLKIS |
| | VL | RVEAEDVGVYYCMQHIEYPITFGQGTK |
| | | LEIK |
| SEQ ID | hVB22 | QVQLVQSGPEVKKPGASVKVSCKASGY |
| NO: 47 | Bq- | TFTNSWMNWVRQRPGKGLEWIGRIYPG |
| | wz5: | DGETIYNGKFRVRVTITADESTSTAYM |
| | VH | ELSSLRSEDTAVYYCARGYDDYSFAYW |
| | | GQGTTVTVSS |
| SEQ ID | hVB22 | DIVMTQSPLSLPVTPGEPASISCRSSK |
| NO: 48 | Bq- | SLLHSNGNTYLYWFQQKPGQAPRLLIY |
| | wz5: | RMSNLASGVPDRFSGSGSGTAFTLKIS |
| | VL | RVEAEDVGVYYCMQHIEYPFTFGQGTK |
| | | LEIK |

U.S. Pat. No. 6,342,220

| | | |
|---|---|---|
| SEQ ID | 74 | MAQVQLQESGGEMKKPGESLKISCKGY |
| NO: 49 | | GYSFATSWIGWVRQVIPGRGLEWMAIM |
| | | YPGNSDTRHNPSFEDQVTMSADTSINT |
| | | AYLQWSSLKASDTAMYYCARAGVAGGA |
| | | FDLWGKGTMVTVSSGGGGSGGGGSGGG |
| | | GSQSVLTQPASVSGSPGQSITISCTGT |
| | | SSGVGGYNYVSWYQQHPGKAPKLLIYG |
| | | NSNRPSGVPDRFSASKSGNTASLTISG |
| | | LQAEDEADYFCSTYAPPGIIMFGGGTK |
| | | LTVLGAA |
| SEQ ID | 75 | MAEVQLVQSGGGLVKPGGSLRLSCAAS |
| NO: 50 | | GFTFSDYYMSWIRQAPGKGLEWVSYIS |
| | | SSGSTIYYADSVKGRFTISRDNSKNTL |
| | | YLQMNSLRAEDTAVYYCARWSGEDAFD |
| | | IWGQGTMVTVSSGGGGSGGGGSGGGGS |
| | | DIVMTQSPSTLSASVGDRVAITCRASE |
| | | GIYHWLAWYQQKPGKAPKLLIYKASSL |
| | | ASGAPSRFSGSGSGADFTLTISSLQPD |
| | | DFATYYCQQYSNYPLTFGGGTKLEVKR |
| | | AA |
| SEQ ID | 76 | MAEVQLVQSGGGVVQPGGSLSLSCAVS |
| NO: 51 | | GITLRTYGMHWVRQAPGKGLEWVAGIS |
| | | FDGRSEYYADSVKGRFTISRDNSKNTL |
| | | YLQMNSLRAEDTAVYYCARDRGSYGMD |
| | | VWGRGTMVTVSSGGGGSGGGGSGGGGS |
| | | DIQMTQSPSTLSASIGDRVTITCRASE |
| | | GIYHWLAWYQQKPGKAPKLLIYKASSL |
| | | ASGAPSRFSGSGSGIDITLTISSLQPD |
| | | DFATYYCQQYSNYPLTFGGGTKLEILR |
| | | AA |
| SEQ ID | 77 | MAQVQLVQSGGGLVRPGGSLSLSCAVS |
| NO: 52 | | GITLRTYGMHWVRQAPGKGLEWVAGIS |
| | | FDGRSEYYADSVQGRFTISRDSSKNTL |
| | | YLQMNSLRAEDTAVYYCARGAHYGFDI |
| | | WGQGTMVTVSSGGGGTGGGGSGGGGSD |
| | | IQMTQSPSTLSASIGDRVTITCRASEG |
| | | IYHWLAWYQQKPGKAPKLLIYKASSLA |
| | | SGAPSRFSGSGSGTDFTLTISSLQPDD |
| | | FATYYCQQYSNYPLTFGGGTELEIKRA |
| | | A |
| SEQ ID | 78 | MAQVQLVESGGGLVKPGGSLRLSCAAS |
| NO: 53 | | GFTFSSHNMNWVRQAPGKGLEWVSSIS |
| | | SSSSYIYYADSVKGRFTISRDNAKNSL |
| | | YLQMNSLRAEDTAVYYCARDRGSTGMD |
| | | VWGRGTLVTVSSGGGGSGGGGSGGGGS |
| | | KIQMTQSPSTLSASIGDRVTITCRASE |
| | | GIYHWLAWYQQKPGKAPKLLIYKASSL |
| | | ASGAPSRFSGSGSGTDFTXTISSLQPD |
| | | DFATYYCQQYSNYPLTFGGGTKLEIKR |
| | | AA |
| SEQ ID | 79 | MAQVQLQQSGPGLVKPSETLSLTCTVS |
| NO: 54 | | GDSISSYYWSWIRQPPGKGLEWIGYIY |
| | | YSGSTNYNPSLKSRVTISVDTSKSQFS |
| | | LKLSSVTAADTAVYYCARGRYFDVWGR |
| | | GTMVIVSSGGGGSGGGGSGGGGSSYVL |
| | | TQPPSVSGSPGQSITISCTGTSSDVGG |
| | | YNYVSWYQQHPGKAPKLMIYEGSKRPS |
| | | GVSNRFSGSKSGNTASLTISGLQAEDE |
| | | ADYYCSSYTTRSTRVFGGGTKLTVLGA |
| | | A |

U.S. Pat. No. 8,034,903

| | | |
|---|---|---|
| SEQ ID | 12E10 | MKHLWFFLLLVAAPRWVLSQVQLQQSG |
| NO: 55 | | PGLVKPSETLSLTCTVSGDSISSYYWS |
| | | WIRQPPGKGLEWIGYIYYSGSTNYNPS |
| | | LKSRVTISVDTSKSQFSLKLSSVTAAD |
| | | TAVYYCARGRYFDVWGRGTMVTVSSGG |
| | | GGSGGGGSGGGGSSYVLTQPPSVSGSP |
| | | GQSITISCTGTSSDVGGYNYVSWYQQH |
| | | PGKAPKLMIYEGSKRPSGVSNRFSGSK |
| | | SGNTASLTISGLQAEDEADYYCSSYTT |
| | | RSTRVFGGGTKLTVLDYKDDDDK |
| SEQ ID | 12B5 | MEFGLSWVFLVALLRGVQCQVQLVQSG |
| NO: 56 | | GGLVRPGGSLSLSCAVSGITLRTYGMH |
| | | WVRQAPGKGLEWVAGISFDGRSEYYAD |
| | | SVQGRFTISRDSSKNTLYLQMNSLRAE |
| | | DTAVYYCARGAHYGFDIWGQGTMVTVS |
| | | SGGGGSGGGGSGGGGSDIQMTQSPSTL |
| | | SASIGDRVTITCRASEGIYHWLAWYQQ |
| | | KPGKAPKLLIYKASSLASGAPSRFSGS |
| | | GSGTDFTLTISSLQPDDFATYYCQQYS |
| | | NYPLTFGGGTKLEIKDYKDDDDK |

US 2012/0269814 A1

| | | |
|---|---|---|
| SEQ ID | Antibody | QIVLTQSPAI MSASPGEKVT |
| NO: 57 | 1 > 42 | ISCSASSSVS YMYWYQQKPG |
| | VL | SSPKPWIYRT SNLASGVPAR |
| | | FSGSGSGTSY SLTISNMEAE |
| | | DAAAYYCQQY HSYPTTFGGG |
| | | TKLEVK |
| SEQ ID | Antibody | EVQLVESGGG LVQPKGSLKL |
| NO: 58 | 1 > 44 | SCAASGFSFN TYAMNWVRQA |
| | VH | PGKGLEWIAH IRSKSNNFAT |
| | | YYADSVKDRF SISRDASENI |
| | | LFLQMNNLKT EDTAMYYCVR |
| | | QGGDFPMDYW GQGTSVTVSS |
| SEQ ID | Antibody | QVQLQQSGPE LVKPGASVKM |
| NO: 59 | 2 > 48 | SCKASGYAFS SSWLNWVRQR |
| | VH | PGKGLEWIGR IYPGDGENHY |
| | | NGKFKGKATL TADKSSSTGY |
| | | MQLSSLTSED SAVYFCASYY |
| | | EGGYWGQGTL ITVSA |
| SEQ ID | Antibody | DIVMTQAAPS IPVTPGESVS |
| NO: 60 | 2 > 46 | ISCRSDKSLL HSNGNTYLFW |
| | VL | FLQRPGQSPQ LLIYRMSNLA |
| | | SGVPDRFSGS GSGTAFTLRI |
| | | SGVEAEDVGV YYCMQHLEYP |
| | | YTFGGGTKLE IK |
| SEQ ID | Antibody | DVQLQESGPG LVKPSQSLSL |
| NO: 61 | 3 > 88 | TCTVTGYSIT IDYTWNWIRQ |
| | VH | FPGNKLEWMG YITYSGSTDY |
| | | NPSLKSRSSI TRDTSMNQFF |
| | | LQLNSVTTED TATYYCARLG |
| | | RRYALDYWGQ GTSVTVSS |
| SEQ ID | Antibody | DIQMTQSSSS FSVSLGDRVT |
| NO: 62 | 3 > 86 | ITCKASEDIY IRLAWYQQKP |
| | VL | GNAPRLLISA ATSLETGIPS |
| | | RFSGSGSGED YTLTITSLQT |
| | | EDVATYYCQQ YWTTPWTFGG |
| | | GTKLEIKR |
| SEQ ID | Antibody | DIVMTQAAPS VPVTPGESVS |
| NO: 63 | 4 > 90 | ISCRSSKSLL HSNGNTYLYW |
| | VL | FLQRPGQSPQ LLIYRMSNLA |
| | | SGVPDRISGS GSGTAFTLRI |
| | | SRVEAEDVGV YYCMQHLEYP |
| | | YTFGGGTKLE IKR |
| SEQ ID | Antibody | QVQLQQSGPE LVKPGASVKI |
| NO: 64 | 4 > 92 | SCKASGYGFS NSWMNWVRQR |
| | VH | PGKGLEWIGR IYPGDGETSY |
| | | NGEFVGKATL TADKSSSTAY |
| | | MHLSSLTSED SAVYFCASYY |
| | | EGGYWGQGTL VTVS |
| SEQ ID | Antibody | DIVMTQAAPS LPVTPGESVS |
| NO: 65 | 5 > 94 | ISCRSSKSLL HSNGNTYLFW |
| | VL | FLQRPCGQSPH LLIYRMSNLA |
| | | SGVPDRFSGS GSGTAFTLRI |
| | | SRVEAEDVGV YYCMQHLEYP |
| | | YTFGGGTKLE IKR |
| SEQ ID | Antibody | QVQLQQSGPE LVKPGASVKI |
| NO: 66 | 5 > 96 | SCKASGYGFS SSWMNWVKQR |
| | VH | PGKGLEWIGR IYPGDGETSY |
| | | NGEFKGKATL TADKSSSTAY |
| | | MQLSSLTSED SAVYFCASYY |
| | | EGGYWGQGTL VTVSA |

TABLE 2
Amino acid sequences of immunoglobulin
constant regions.

| | | |
|---|---|---|
| SEQ ID | Human CH2, | DKTHTCPPCPAPELLGGPSVFLF |
| NO: 67 | CH3 knob | PPKPKDTLMISRTPEVTCVVVDV |
| | | SHEDPEVKENWYVDGVEVHNAKT |
| | | KPREEQYNSTYRVVSVLTVLHQD |
| | | WLNGKEYKCKVSNKALPAPIEKT |
| | | ISKAKGQPREPQVYTLPPCREEM |
| | | TKNQVSLWCLVKGFYPSDIAVEW |
| | | ESNGQPENNYKTTPPVLDSDGSF |
| | | FLYSKLTVDKSRWQQGNVFSCSV |
| | | MHEALHNHYTQKSLSLSPGK |
| SEQ ID | Human CH2, | DKTHTCPPCPAPELLGGPSVFLF |
| NO: 68 | CH3 hole | PPKPKDTLMISRTPEVTCVVVDV |
| | | SHEDPEVKFNWYVDCrVEVHNAK |
| | | TKPREEQYNSTYRVVSVLTVLHQ |
| | | DWLNGKEYKCKVSNKALPAPIEK |
| | | TISKAKGQPREPQVCTLPPSREE |
| | | MTKNQVSLSCAVKGFYPSDIAVE |
| | | WESNGQPENNYKTTPPVLDSDGS |
| | | FFLVSKLTVDKSRWQQGNVFSCS |
| | | VMHEALHNHYTQKSLSLSPGK |
| SEQ ID | CH1 | ASTKGPSVFPLAPSSKSTSGGTA |
| NO: 69 | | ALGCLVKDYFPEPVTVSWNSGAL |
| | | TSGVHTFPAVLQSSGLYSLSSVV |
| | | TVPSSSLGTQTYICNVNHKPSNT |
| | | KVDKRVEPKSC |
| SEQ ID | CL | RTVAAPSVITFPPSDEQLKSGTA |
| NO: 70 | (kappa) | SVVCLLNNFYPREAKVQWKVDNA |
| | | LQSGNSQESVTEQDSKDSTYSLS |
| | | STLTLSKADYEKHKVYACEVTHQ |
| | | GLSSPVTKSFNRGEC |
| SEQ ID | CL | GQPKANPTVTLFPPSSEELQANK |
| NO: 71 | (lambda) | ATLVCLISDFYPGAVTVAWKADG |
| | | SPVKAGVETTKPSKQSNNKYAAS |
| | | SYLSLTPEQWKSHRSYSCQVTHE |
| | | GSTVEKTVAPTECS |

### Phosphatase-Targeting Moieties

In some embodiments, the multispecific molecule disclosed herein comprises a phosphatase-targeting moiety. In one embodiment, the multispecific molecule comprises a first targeting moiety that binds to MPL (e.g., an MPL-targeting moiety disclosed herein) and a second targeting moiety that binds to a phosphatase, e.g., a protein tyrosine phosphatase (PTP), e.g., a receptor protein tyrosine phosphatase (RPTP). In one embodiment, the phosphatase and MPL are expressed in a same cell. In one embodiment, the phosphatase can dephosphorylate MPL or a molecule that interacts directly or indirectly with MPL (e.g., a tyrosine kinase that interacts directly or indirectly with MPL, e.g., JAK2 or Src). In one embodiment, the phosphatase is selected from the group consisting of CD45, RPTPμ, RPTPκ, RPTPρ, RPTPλ, leukocyte antigen-related tyrosine phosphatase (LAR), RPTPσ, RPTPδ, RPTPβ, CD148, SAP1, RPTPO, RPTPQ/PTPS31, RPTPα, RPTPε, RPTPζ, RPTPγ, PC-PTP, IA2, and IA2β. In one embodiment, the phosphatase is CD45. In one embodiment, the phosphatase is CD148. In one embodiment, the phosphatase is LAR.

### CD45-Targeting Moieties

CD45, also known as receptor-type tyrosine-protein phosphatase C, Leukocyte common antigen (LCA), or T200, is encoded by the gene PTPRC. CD45 has several isoforms produced by alternative slicing, e.g., CD45RA, CD45RB, CD45RC, CD45RAB, CD45RAC, CD45RBC, CD45RO, and CD45R (ABC).

In one embodiment, the multispecific molecule disclosed herein comprises a targeting moiety that binds to a CD45 isoform. In one embodiment, the multispecific molecule disclosed herein comprises a targeting moiety that binds specifically to one CD45 isoform. In one embodiment, the multispecific molecule disclosed herein comprises a targeting moiety that binds to more than one CD45 isoform. In one embodiment, the multispecific molecule disclosed herein comprises a targeting moiety that binds to all the CD45 isoforms.

Exemplary CD45 Targeting Moieties

Exemplary CD45-targeting moieties have been disclosed in: e.g., U.S. Pat. Nos. 5,273,738, 7,265,212, 7,825,222, US20040096901, WO2005026210, WO2016187514, and WO2017009473, herein incorporated by reference in their entireties.

In one embodiment, the CD45-targeting moiety comprises an antibody molecule (e.g., Fab or scFv) that binds to CD45.

In one embodiment, the CD45-targeting moiety comprises one or more of the CDR sequences, the heavy or light chain variable region sequence, or the heavy or light chain sequence of the antibody BC8 or 9.4, or a sequence sharing 70, 75, 80, 85, 90, or 99% identity thereof. The hybridomas producing the antibody BC8 or 9.4 were deposited at the ATCC under accession numbers HB10507 and HB10508, respectively. In one embodiment, the CD45-targeting moiety comprises one or more of the CDR sequences, the heavy or light chain variable region sequence, or the heavy or light chain sequence of the antibody clone 30-F11 or 5B1 (Miltenyi Biotec), or a sequence sharing 70, 75, 80, 85, 90, or 99% identity thereof. In one embodiment, the CD45-targeting moiety comprises one or more of the CDR sequences, the heavy or light chain variable region sequence, or the heavy or light chain sequence of the antibody YTH-24, YTH-24/54, YTH-25.4, or YTH-54.12, or a sequence sharing 70, 75, 80, 85, 90, or 99% identity thereof. In one embodiment, the CD45-targeting moiety comprises one or more of the CDR sequences, the heavy or light chain variable region sequence, or the heavy or light chain sequence of the antibody YAML568, or a sequence sharing 70, 75, 80, 85, 90, or 99% identity thereof.

In some embodiments, the CD45-targeting moiety comprises one, two, or three CDRs from any of the heavy chain variable domain sequences of Table 3, or a closely related CDR, e.g., CDRs which have at least one amino acid alteration, but not more than two, three or four alterations (e.g., substitutions, deletions, or insertions, e.g., conservative substitutions) from any the CDR sequences of Table 3. In some embodiments, the CD45-targeting moiety comprises a heavy chain variable domain sequence chosen from any of the amino acid sequences of Table 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

Alternatively, or in combination with the heavy chain to CD45 disclosed herein, the CD45-targeting moiety comprises one, two, or three CDRs from any of the light chain variable domain sequences of Table 3, or a closely related CDR, e.g., CDRs which have at least one amino acid alteration, but not more than two, three or four alterations (e.g., substitutions, deletions, or insertions, e.g., conservative substitutions) from any of the CDR sequence of Table 3. In some embodiments, the CD45-targeting moiety comprises a light chain variable domain sequence chosen from any of the amino acid sequences of Table 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

TABLE 3
Amino acid sequences of variable regions of
exemplary anti-CD45 antibodies.

| | VH | VL |
|---|---|---|
| Ab 1 | EVQLVESGGGLVQPGGSLRLSCAASGFSFSA | DIQMTQSPSTLSASVGDRVTITCQASQ |
| | GYWICWVRQAPGKGLEWIACTYAGRSGSTYY | SISNWLAWYQQKPGKAPKLLIYQASKL |
| | ANWVNGRFTISKDSAKTSVYLQMNSLRAEDT | ASGVPSRFSGSGSGTEYTLTISSLQPD |
| | AVYYCARGNAGVAVGALWGRGTLVTVSS | DFATYYCQSYYDSGSNVFFAFGGGTK |
| | (SEQ ID NO: 72) | VEIK (SEQ ID NO: 76) |
| Ab 2 | EVQLVESGGGLVQPGGSLRLSCAASGFSFSA | DIQMTQSPSTLSASVGDRVTITCQASQ |
| | GYWISWVRQAPGKGLEWIASTYAGRSGSTYY | SISNWLAWYQQKPGKAPKLLIYQASKL |
| | ANWVNGRFTISKDSAKTSVYLQMNSLRAEDT | ASGVPSRFSGSGSGTEYTLTISSLQPD |
| | AVYYCARGNAGVAVGALWGRGTLVTVSS | DFATYYCQSYYDSGSNVFFAFGGGTK |
| | (SEQ ID NO: 73) | VEIK (SEQ ID NO: 76) |
| Ab 3 | EVTLKESGPALVKPTQTLTLTCTASGFSFSAG | DIQMTQSPSTLSASVGDRVTITCQASQ |
| | YWICWVRQPPGKGLEWIACTYAGRSGSTYYA | SISNWLAWYQQKPGKAPKLLIYQASKL |
| | NWVNGRFTISKDSSKTQVVLTMTNMDPVDT | ASGVPSRFSGSGSGTEYTLTISSLQPD |
| | ATYYCARGNAGVAVGALWGRGTLVTVSS | DFATYYCQSYYDSGSNVFFAFGGGTK |
| | (SEQ ID NO: 74) | VEIK (SEQ ID NO: 76) |
| Ab 4 | EVTLKESGPALVKPTQTLTLTCTASGFSFSAG | DIQMTQSPSTLSASVGDRVTITCQASQ |
| | YWISWVRQPPGKGLEWIASTYAGRSGSTYYA | SISNWLAWYQQKPGKAPKLLIYQASKL |
| | NWVNGRFTISKDSSKTQVVLTMTNMDPVDT | ASGVPSRFSGSGSGTEYTLTISSLQPD |
| | ATYYCARGNAGVAVGALWGRGTLVTVSS | DFATYYCQSYYDSGSNVFFAFGGGTK |
| | (SEQ ID NO: 75) | VEIK (SEQ ID NO: 76) |
| Ab 5 | EVQLVESGGGLVQPGGSLRLSCAASGFSFSAG | DIQMTQSPSTLSASVGDRVTITCQASQ |
| | YWICWVRQAPGKGLEWIACIYAGSSGSTYYA | SISSWLSWYQQKPGKAPKLLIYGASNL |
| | SWAKGRFITSKDSAKTSVYLQMNSLRAEDTA | ASGVPSRFSGSGSGTQFTLTISSLQPD |
| | VYYCARGNAGVAVGALWGRGTLVTVSS | DFATYYCQSYYDSGSSVFFNFGGGTK |
| | (SEQ ID NO: 77) | VEIK (SEQ ID NO: 81) |
| Ab 6 | EVQLVESGGGLVQPGGSLRLSCAASGFSFSAG | DIQMTQSPSTLSASVGDRVTITCQASQ |
| | YWISWVRQAPGKGLEWIASIYAGSSGSTYYA | SISSWLSWYQQKPGKAPKLLIYGASNL |
| | SWAKGRFTISKDSAKTSVYLQMNSLRAEDTA | ASGVPSRFSGSGSGTQFTLTISSLQPD |
| | VYYCARGNAGVAVGALWGRGTLVTVSS | DFATYYCQSYYDSGSSVFFNFGGGTK |
| | (SEQ ID NO: 78) | VEIK (SEQ ID NO: 81) |
| Ab 7 | EVTLKESGPALVKPTQTLTLTCTASGFSFSAG | DIQMTQSPSTLSASVGDRVTITCQASQ |
| | YWICWVRQPPGKGLEWIACIYAGSSGSTYYA | SISSWLSWYQQKPGKAPKLLIYGASNL |
| | SWAKGRFTISKDSSKTQVVLTMTNMDPVDTA | ASGVPSRFSGSGSGTQFTLTISSLQPD |
| | TYYCARGNAGVAVGALWGRGTLVTVSS | DFATYYCQSYYDSGSSVFFNFGGGTK |
| | (SEQ ID NO: 79) | VEIK (SEQ ID NO: 81) |
| Ab 8 | EVTLKESGPALVKPTQTLTLTCTASGFSFSAG | DIQMTQSPSTLSASVGDRVTITCQASQ |
| | YWISWVRQPPGKGLEWIASIYAGSSGSTYYAS | SISSWLSWYQQKPGKAPKLLIYGASNL |
| | WAKGRFTISKDSSKTQVVLTMTNMDPVDTAT | ASGVPSRFSGSGSGTQFTLTISSLQPD |
| | YYCARGNAGVAVGALWGRGTLVTVSS | DFATYYCQSYYDSGSSVFFNFGGGTK |
| | (SEQ ID NO: 80) | VEIK (SEQ ID NO: 81) |
| Ab 9 | EVQLVESGGGLVQPGGSLRLSCAASGVSFSSS | DIVMTQSPSSVSASVGDRVTITCQASQ |
| | YWIYWVRQAPGKGLEWIACIYTGSSGSTYYA | SFYNLLAWYQQKPGKAPKLLIYDASDL |
| | SWAKGRFTVSEDSAKTSVYLQMNSLRAEDTA | ASGVPSRFKGSGSGTDFTLTISSLQPE |
| | VYYCARASAWTYGMDLWGRGTLVTVSS | DFATYYCQSADGSSYAFGGGTKVEIK |
| | (SEQ ID NO: 82) | (SEQ ID NO: 86) |
| Ab 10 | EVQLVESGGGLVQPGGSLRLSCAASGVSFSSS | DIVMTQSPSSVSASVGDRVTITCQASQ |
| | YWIYWVRQAPGKGLEWIASIYTGSSGSTYYA | SFYNLLAWYQQKPGKAPKLLIYDASDL |
| | SWAKGRFTVSEDSAKTSVYLQMNSLRAEDTA | ASGVPSRFKGSGSGTDFTLTISSLQPE |
| | VYYCARASAWTYGMDLWGRGTLVTVSS | DFATYYCQSADGSSYAFGGGTKVEIK |
| | (SEQ ID NO: 83) | (SEQ ID NO: 86) |
| Ab 11 | EVQLQESGPGLVKPSQTLSLTCTASGVSFSSS | DIVMTQSPSSVSASVGDRVTITCQASQ |
| | YWIYWVRQHPGKGLEWIACIYTGSSGSTYYAS | SFYNLLAWYQQKPGKAPKLLIYDASDL |
| | WAKGRFTVSEDSSKTQVSLKLSSVTAADTAV | ASGVPSRFKGSGSGTDFTLTISSLQPE |
| | YYCARASAWTYGMDLWGRGTLVTVSS | DFATYYCQSADGSSYAFGGGTKVEIK |
| | (SEQ ID NO:84) | (SEQ ID NO: 86) |
| Ab 12 | EVQLQESGPGLVKPSQTLSLTCTASGVSFSSS | DIVMTQSPSSVSASVGDRVTITCQAS |
| | YWIYWVRQHPGKGLEWIASIYTGSSGSTYYAS | QSFYNLLAWYQQKPGKAPKLLIYDAS |
| | WAKGRFTVSEDSSKTQVSLKLSSVTAADTAV | DLASGVPSRFKGSGSGTDFTLTISSL |
| | YYCARASAWTYGMDLWGRGTLVTVSS | QPEDFATYYCQSADGSSYAFGGGTKVE |
| | (SEQ ID NO:85) | IK |
| | | (SEQ ID NO: 86) |
| Ab 13 | EVQLVESGGGLVQPGGSLRLSCAASGVSFSSS | DIQMTQSPSSVSASVGDRVTITCQAS |
| | YWIYWVRQAPGKGLEWIACIYTGSSGSTYYA | QSFYNLLAWYQQKPGKAPKLLIYDAS |
| | SWAKGRFTVSEDSAKTSVYLQMNSLRAEDTA | DLASGVPSRFSGSGSGTDFTLTISSL |
| | VYYCARASAWTYGMDLWGRGTLVTVSS | QPEDFATYYCQSADGSSYAFGGGTKV |
| | (SEQ ID NO: 82) | EIK |
| | | (SEQ ID NO:87) |
| Ab 14 | EVQLVESGGGLVQPGGSLRLSCAASGVSFSSS | DIQMTQSPSSVSASVGDRVTITCQAS |
| | YWIYWVRQAPGKGLEWIASIYTGSSGSTYYA | QSFYNLLAWYQQKPGKAPKLLIYDAS |
| | SWAKGRFTVSEDSAKTSVYLQMNSLRAEDTA | DLASGVPSRFSGSGSGTDFTLTISSL |
| | VYYCARASAWTYGMDLWGRGTLVTVSS | QPEDFATYYCQSADGSSYAFGGGTKV |
| | (SEQ ID NO: 83) | EIK |
| | | (SEQ ID NO:87) |
| Ab 15 | EVQLQESGPGLVKPSQTLSLTCTASGVSFSSS | DIQMTQSPSSVSASVGDRVTITCQAS |
| | YWIYWVRQHPGKGLEWIACIYTGSSGSTYYAS | QSFYNLLAWYQQKPGKAPKLLIYDAS |
| | WAKGRFTVSEDSSKTQVSLKLSSVTAADTAV | DLASGVPSRFSGSGSGTDFTLTISSL |
| | YYCARASAWTYGMDLWGRGTLVTVSS | QPEDFATYYCQSADGSSYAFGGGTKV |
| | (SEQ ID NO: 84) | EIK |
| | | (SEQ ID NO:87) |
| Ab 16 | EVQLQESGPGLVKPSQTLSLTCTASGVSESSS | DIQMTQSPSSVSASVGDRVTITCQAS |
| | YWIYWVRQHPGKGLEWIASIYTGSSGSTYYAS | QSFYNLLAWYQQKPGKAPKLLIYDAS |
| | WAKGRFTVSEDSSKTQVSLKLSSVTAADTAV | DLASGVPSRFSGSGSGTDFTLTISSL |
| | YYCARASAWTYGMDLWGRGTLVTVSS | QPEDFATYYCQSADGSSYAFGGGTKV |
| | (SEQ ID NO: 85) | EIK |
| | | (SEQ ID NO:87) |
| Ab 17 | EVQLVESGGGLVKPGGSLRLSCAASGFSFSG | AQVLTQSPSSLSASVGDRVTITCQAS |
| | NYYMCWVRQAPGKGLEWIGCLYTGSSGSTYY | QSVYNNNNLSWYQQKPGKAPKLLIYD |
| | ASWAKGRFTISKDSAKTSVYLQMNSLRAEDT | ASKLASGVPSRFSGSGSGTQFTLTIS |
| | AVYYCARDLGYEIDGYGGLWGQGTLVTVSS | SLQPEDFATYYCLGGYYSSGWYFAFG |
| | (SEQ ID NO: 88) | GGTKVEIK |
| | | (SEQ ID NO: 92) |
| Ab 18 | EVQLVESGGGLVKPGGSLRLSCAASGFSFSG | AQVLTQSPSSLSASVGDRVTITCQAS |
| | NYYMSWVRQAPGKGLEWIGSLYTGSSGSTYY | QSVYNNNNLSWYQQKPGKAPKLLIYD |
| | ASWAKGRFTISKDSAKTSVYLQMNSLRAEDT | ASKLASGVPSRFSGSGSGTQFTLTIS |
| | AVYYCARDLGYEIDGYGGLWGQGTLVTVSS | SLQPEDFATYYCLGGYYSSGWYFAFG |
| | (SEQ ID NO: 89) | GGTKVEIK |
| | | (SEQ ID NO:92) |
| Ab 19 | EVQLQESGPGLVKPSGTLSLTCAASGFSFSG | AQVLTQSPSSLSASVGDRVTITCQAS |
| | NYYMCWVRQPPGKGLEWIGCLYTGSSGSTYY | QSVYNNNNLSWYQQKPGKAPKLLIYD |
| | ASWAKGRVTISKDSSKTQVSLKLSSVTAADT | ASKLASGVPSRFSGSGSGTQFTLTIS |
| | AVYYCARDLGYEIDGYGGLWGQGTLVTVSS | SLQPEDFATYYCLGGYYSSGWYFAFG |
| | (SEQ ID NO: 90) | GGTKVEIK |
| | | (SEQ ID NO:92) |
| Ab 20 | EVQLQESGPGLVKPSGTLSLTCAASGFSFSGN | AQVLTQSPSSLSASVGDRVTITCQAS |
| | YYMSWVRQPPGKGLEWIGSLYTGSSGSTYYA | QSVYNNNNLSWYQQKPGKAPKLLIYD |
| | SWAKGRVTISKDSSKTQVSLKLSSVTAADTA | ASKLASGVPSRFSGSGSGTQFTLTIS |
| | VYYCARDLGYEIDGYGGLWGQGTLVTVSS | SLQPEDFATYYCLGGYYSSGWYFAFGG |
| | (SEQ ID NO: 91) | GTKVEIK |
| | | (SEQ ID NO: 92) |
| Ab 21 | EVQLVESGGGLVKPGGSLRLSCAASGFSFSG | AQVLTQSPSSLSASVGDRVTITCQAS |
| | NYYMCWVRQAPGKGLEWIGCLYTGSSGSTYY | QSVYNNNNLAWYQQKPGKAPKLLIYD |
| | ASWAKGRFTISKDSAKTSVYLQMNSLRAEDT | ASKLASGVPSRFSGSGSGTQFTLTIS |
| | AVYYCARDLGYEIDGYGGLWGQGTLVTVSS | SLQPEDFATYYCLGGYYSSGWYFAFG |
| | (SEQ ID NO: 88) | GGTKVEIK |
| | | (SEQ ID NO: 93) |
| Ab 22 | EVQLVESGGGLVKPGGSLRLSCAASGFSFSG | AQVLTQSPSSLSASVGDRVTITCQAS |
| | NYYMSWVRQAPGKGLEWIGSLYTGSSGSTYY | QSVYNNNNLAWYQQKPGKAPKLLIYD |
| | ASWAKGRPTISKDSAKTSVYLQMNSLRAEDT | ASKLASGVPSRFSGSGSGTQFTLTIS |
| | AVYYCARDLGYEIDGYGGLWGQGTLVTVSS | SLQPEDFATYYCLGGYYSSGWYFAFG |
| | (SEQ ID NO: 89) | GGTKVEIK |
| | | (SEQ ID NO: 93) |
| Ab 23 | EVQLQESGPGLVKPSGTLSLTCAASGFSFSG | AQVLTQSPSSLSASVGDRVTITCQAS |
| | NYYMCWVRQPPGKGLEWIGCLYTGSSGSTYY | QSVYNNNNLAWYQQKPGKAPKLLIYD |
| | ASWAKGRVTISKDSSKTQVSLKLSSVTAADT | ASKLASGVPSRFSGSGSGTQFTLTIS |
| | AVYYCARDLGYEIDGYGGLWGQGTLVTVSS | SLQPEDFATYYCLGGYYSSGWYFAFG |
| | (SEQ ID NO: 90) | GGTKVEIK |
| | | (SEQ ID NO: 93) |
| Ab 24 | EVQLQESGPGLVKPSGTLSLTCAASGFSFSG | AQVLTQSPSSLSASVGDRVTITCQAS |
| | NYYMSWVRQPPGKGLEWIGSLYTGSSGSTYY | QSVYNNNNLAWYQQKPGKAPKLLIYD |
| | ASWAKGRVTISKDSSKTQVSLKLSSVAADTA | ASKLASGVPSRFSGSGSGTQFTLTIS |
| | VYYCARDLGYEIDGYGGLWGQGTLVTVSS | SLQPEDFATYYCLGGYYSSGWYFAFG |
| | (SEQ ID NO: 91) | GGTKVEIK |
| | | (SEQ ID NO: 93) |

### CD148-Targeting Moieties

CD148, also known as Receptor-type tyrosine-protein phosphatase eta (R-PTP-eta) or density-enhanced phosphatase 1 (DEP-1), is encoded by the gene PTPRJ.

In one embodiment, the multispecific molecule disclosed herein comprises a targeting moiety that binds to CD148.

Exemplary CD148 Targeting Moieties

Exemplary CD148-targeting moieties have been disclosed in: e.g., US20090263383 and U.S. Pat. No. 7,195,762, herein incorporated by reference in their entireties.

In one embodiment, the CD148-targeting moiety comprises an antibody molecule (e.g., Fab or scFv) that binds to CD148.

In some embodiments, the CD148-targeting moiety comprises one, two, or three CDRs from any of the heavy chain variable domain sequences of Table 4, or a closely related CDR, e.g., CDRs which have at least one amino acid alteration, but not more than two, three or four alterations (e.g., substitutions, deletions, or insertions, e.g., conservative substitutions) from any the CDR sequences of Table 4. In some embodiments, the CD148-targeting moiety comprises a heavy chain variable domain sequence chosen from any of the amino acid sequences of Table 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

Alternatively, or in combination with the heavy chain to CD148 disclosed herein, the CD148-targeting moiety comprises one, two, or three CDRs from any of the light chain variable domain sequences of Table 4, or a closely related CDR, e.g., CDRs which have at least one amino acid alteration, but not more than two, three or four alterations (e.g., substitutions, deletions, or insertions, e.g., conservative substitutions) from any of the CDR sequence of Table 4. In some embodiments, the CD148-targeting moiety comprises a light chain variable domain sequence chosen from any of the amino acid sequences of Table 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

TABLE 4
Amino acid sequences of variable regions
of exemplary anti-CD148 antibodies.

| | VH | VL |
|---|---|---|
| Ab 25 | EVQLLESGGGLVQPGGS | QAVLTQPSSVSGA |
| | LRLSCAASGFTFSSYAM | FGQRVTISCTGSS |
| | SWVRQAPGKGLEWVSAI | SNIGAGYDVHWYQ |
| | SGSGGSTYYADSVKGRF | QLPGTAPKLLIYG |
| | TISRDXSKNTLYLQMNS | NSNRPSGVPDRFS |
| | LRAEDTAVYYCARGRTE | GSKSGTSASLAVT |
| | VATPGAYWGQGTMVTVS | GLQAEDEADYYCQ |
| | S | SYDSSLSDVVFGG |
| | (SEQ ID NO: 94) | GTKLTVL |
| | | (SEQ ID NO: 95) |
| Ab 26 | EVQLLESGGGLVQPGG | EIVMTQSPSSLPA |
| | SLRLSCAASGFTFSSY | SVGDRVTITCRAS |
| | AMSWVRQAPGKGLEWV | QNIKTYLHWYQQK |
| | SAISGSGGSTYYADSV | PGKAPNLLIYAAS |
| | KGRFTISRDNSKNTLY | NLQIGVPSRFSGS |
| | LQMNSLRAEDTAVYYC | GSGTDFTLTISSL |
| | ARYRDYGGNSHLFDYW | QPEDFATYFCQQS |
| | GQGTTVTVSS | YITPPTFGQGTRL |
| | (SEQ ID NO: 96) | LIK |
| | | (SEQ ID NO: 97) |
| Ab 27 | GVQLVQSGAEVKKPGA | DIQMTQSPSTLSA |
| | SVKVSCKASGYTFTSY | SIGDRVTITCRAS |
| | YMHWVRQAPGQGLEWM | EGIYHWLAWYQQK |
| | GIINPSGGSTSYAQKF | PGKAPKLLIYKAS |
| | QGRVTMTRDTSTSTVY | SLASGAPSRFSGS |
| | MELSSLRSEDTAVYYC | GSGTDFTLTISSL |
| | ARRVISGAFDIWGQGT | QPDDFATYYCQQY |
| | MVTVSS | SNYPLTFGGGTKL |
| | (SEQ ID NO:98) | EIK |
| | | (SEQ ID NO: 99) |
| Ab 28 | QVQLVQSGAEVKKPGA | SSELTQDPAVSVA |
| | SVKVSCKASGYTFTSY | LGQTVRITCQGDS |
| | YMHWVRQAPGQGLEWM | LRSYYTNWFQQKP |
| | GIINPSDGSTRYVEKF | GQAPLLVVYAKNK |
| | QGRVTMTRDTSTSTVY | RPSGTPDRFSGSS |
| | MELSSLRSEDTAVYFC | SGNTASLTITGAQ |
| | ARGMGPGPHYHFYMDV | AEDEADYYCHSRD |
| | WGKGTMVTVSS | SGGNHVLFGGGT |
| | (SEQ ID NO: 100) | KLTVL |
| | | (SEQ ID NO: 101) |
| Ab 29 | QVQLVQSGAEVKKPGA | SSELTQDPAVSV |
| | SVKVSCKASGYTFTGQ | ALGQTVRITCQG |
| | YIHWVRQAPGQGLEWM | DSLRSYYASWYQ |
| | GWISAYNGYTDYAQKV | QKPGQAPVLVIY |
| | QGRVTMTTDTSTSTAY | GKNNRPSGIPDR |
| | MELRSLRSDDTAVYYC | FSGSSSGNTASL |
| | AREVWPVAAADTFSVF | TITGAQAEDEAD |
| | DIWGRGTLVTVSS | YYCNSRDSSGNH |
| | (SEQ ID NO: 102) | VVFGGGTKLTVL |
| | | (SEQ ID NO: 103) |
| Ab 30 | EVQLLESGGGLVQPGG | QSVLTQPPSAS |
| | SLRLSCAASGFTFSSY | GTPGQRVTISC |
| | AMSWVRQAPGKGLEWV | SGSSSNVGSNF |
| | SAISGSGGSTYYADSV | VYWYQQFPGTA |
| | KGRFTISRDNSKNTLY | PKLLIYRNNQR |
| | LQMNSLRAEDTAVYYC | PSGVPDRFSGS |
| | ARDGTTGLHDSWGQGT | KSGTSASLAIS |
| | MVTVSS | GLRSLDLADYY |
| | (SEQ ID NO: 104) | CAAWDDTLNGH |
| | | YVFGGGTKLTVL |
| | | (SEQ ID NO: 105) |
| Ab 31 | EVQLVQSGAEVKKPG | RSVLTQPPSVSA |
| | ESLKISCKGYGYDFS | APGQKVTISCSG |
| | RDWIAWVRQMPGKGL | STSNIGNNYVSW |
| | EWMGIIYPGDSDTRY | YQQHPGKAPKLM |
| | SPSFEGQVTISADKS | IYDVSKRPSGVP |
| | ISTAYLQWRSLKASD | DRFSGSKSGNSA |
| | TAMYYCARQRRLGWF | SLDISGLQSEDE |
| | DPWGQGTMVTVSS | ADYYCAAWDDSL |
| | (SEQ ID NO: 106) | SEFLFGTGTKLT |
| | | VL |
| | | (SEQ ID NO: 107) |
| Ab 32 | EVQLLESGGGLVQPG | SYVLTQPPSASG |
| | GSLRLSCAASGFTFS | TPGQRVTISCSG |
| | SYAMSWVRQAPGKGL | SSSNIGSNYVYW |
| | EWVSAISGSGGSTYY | YQQLPGTAPKLL |
| | ADSVKGRFTISRDNS | IYRNNQRPSGVP |
| | KNTLYLQMNSLRAED | DRFSGSKSGTSA |
| | TAVYYCARHLPSGSS | SLAISGLQSEDE |
| | SSWAFDSWGRGTTVT | ADYYCEAWDDNV |
| | VSS | DCiPVFGGGTKL |
| | (SEQ ID NO: 108) | TVL |
| | | (SEQ ID NO: 109) |

### LAR-Targeting Moieties

Leukocyte antigen-related tyrosine phosphatase (LAR), also known as Receptor-type tyrosine-protein phosphatase F, is encoded by the gene PTPRF.

In one embodiment, the multispecific molecule disclosed herein comprises a targeting moiety that binds to LAR.

Exemplary LAR Targeting Moieties

Exemplary LAR-targeting moieties have been disclosed in: U.S. Pat. Nos. 7,858,086, 6,846,912, and 6,852,486, herein incorporated by reference in their entireties.

In one embodiment, the LAR-targeting moiety comprises an antibody molecule (e.g., Fab or scFv) that binds to LAR.

In some embodiments, the LAR-targeting moiety comprises one, two, or three CDRs from any of the heavy chain variable domain sequences of Table 5, or a closely related CDR, e.g., CDRs which have at least one amino acid alteration, but not more than two, three or four alterations (e.g., substitutions, deletions, or insertions, e.g., conservative substitutions) from any the CDR sequences of Table 5. In some embodiments, the LAR-targeting moiety comprises a heavy chain variable domain sequence chosen from any of the amino acid sequences of Table 5, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

Alternatively, or in combination with the heavy chain to LAR disclosed herein, the LAR-targeting moiety comprises one, two, or three CDRs from any of the light chain variable domain sequences of Table 5, or a closely related CDR, e.g., CDRs which have at least one amino acid alteration, but not more than two, three or four alterations (e.g., substitutions, deletions, or insertions, e.g., conservative substitutions) from any of the CDR sequence of Table 5. In some embodiments, the LAR-targeting moiety comprises a light chain variable domain sequence chosen from any of the amino acid sequences of Table 5, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

TABLE 5
Amino acid sequences of variable regions
of exemplary anti-LAR antibodies.

| VH | VL | |
|---|---|---|
| Ab 33 | EVQLVESGGGLVQPGGSLRL | TQSPSSLSASVGDRVTITCR |
| | SCAASGFTFSSYWMHWVRQA | ASQSISSYLNWYQQKPGKAP |
| | PGKGLVWVSRINSDGSSTSY | KLLIYAASSLQSGVPSRFSG |
| | ADSVKGRFTISRDNAKNTLY | SGSGTDFTLTISSLQPEDFA |
| | LQMNSLRAEDTAVYYCARDD | TYYCQQSYSTPPTFGQGTKV |
| | TPTSDYGFDSWGQGTLVTVS | |
| | S | (SEQ ID NO: 111) |
| | (SEQ ID NO: 110) | |

### TGFβ Antagonists

The present disclosure also provides multispecific molecules that comprise a TGFβ antagonist, e.g., a polypeptide comprising a TGFβ receptor, or functional fragment or variant thereof, that is capable of binding TGFβ. In one embodiment, the TGFβ antagonist comprises an extracellular domain of a TGFβ receptor. In one embodiment, the TGFβ antagonist comprises an extracellular domain of TGFβ receptor type I, or functional fragment or variant thereof. TGFβ receptor type I, also known as Activin receptor-like kinase 5 (ALK-5) or Serine/threonine-protein kinase receptor R4 (SKR4), is encoded by the gene TGFBR1. In one embodiment, the TGFβ antagonist comprises an extracellular domain of TGFβ receptor type II, or functional fragment or variant thereof. TGFβ receptor type II is encoded by the gene TGFBR2. In one embodiment, the TGFβ antagonist binds to a TGFβ selected from the group consisting of TGFβ1, TGFβ2, and TGFβ3. In one embodiment, the TGFβ antagonist binds to all of TGFβ1, TGFβ2, and TGFβ3. In one embodiment, the TGFβ antagonist binds TGFβ1 and TGFβ3.

Exemplary TGFβ Antagonists

Exemplary TGFβ antagonists have been disclosed in: U.S. Pat. Nos. 8,993,524, 9,676,863, 9,611,306, 8,318,135, and WO2017037634, herein incorporated by reference in their entireties.

In some embodiments, the TGFβ antagonist comprises any amino acid sequence of Table 6, or an amino acid sequence substantially identical thereto (e.g., 75%, 8-0%, 85%, 90%, 95%, or 99.9% identical thereto), or having at least one amino acid alteration, but not more than five, ten, fifteen, or twenty alterations (e.g., substitutions, deletions, or insertions, e.g., conservative substitutions).

TABLE 6
Amino acid sequences of exemplary
TGFβ antagonists.

| | | |
|---|---|---|
| SEQ ID NO: | TGFβ | IPPHVQKSVNNDMIVTDNNGA |
| 112 | receptor | VKFPQLCKFCDVRFSTCDNQK |
| | type II | SCMSNCSITSICEKPQEVCVA |
| | isoform 1 | VWRKNDENITLETVCHDPKLP |
| | ECD | YHDFILEDAASPKCIMKEKKK |
| | | PGETFFMCSCSSDECNDNIIF |
| | | SEEYNTSNPD |
| SEQ ID NO: | TGFβ | IPPHVQKSDVEMEAQKDEIIC |
| 113 | receptor | PSCNRTAHPLRHINNDMIVTD |
| | type II | NNGAVKFPQLCKFCDVRFSTC |
| | isoform 2 | DNQKSCMSNCSITSICEKPQE |
| | ECD | VCVAVWRKNDENITLETVCHD |
| | | PKLPYHDFILEDAASPKCIMK |
| | | EKKKPGETFFMCSCSSDECND |
| | | NIIFSEEYNTSNPD |
| SEQ ID NO: | TGFβ | IPPHVQKSVNNDMIVTDNNGA |
| 114 | receptor | VKFPQLCKFCDVRFSTCDNQK |
| | type II | SCMSNCSITSICEKPQEVCVA |
| | isoform 1 | VWRKNDENITLETVCHDPKLP |
| | ECD dimer | YHDFILEDAASPKCIMKEKKK |
| | | PGETFFMCSCSSDECNDNIIF |
| | | SEEYNTSNPDIPPHVQKSVNN |
| | | DMIVTDNNGAVKFPQLCKFCD |
| | | VRFSTCDNQKSCMSNCSITSI |
| | | CEKPQEVCVAVWRKNDENITL |
| | | ETVCHDPKLPYHDFILEDAAS |
| | | PKCTMKEKKKPGETFFMCSCS |
| | | SDECNDNIIFSEEYNTSNPD |
| SEQ ID NO: | TGFβ | IPPHVQKSDVEMEAQKDEIIC |
| 115 | receptor | PSCNRTAHPLRHINNDMIVTD |
| | type II | NNGAVKFPQLCKFCDVRFSTC |
| | isoform 2 | DNQKSCMSNCSITSICEKPQE |
| | ECD dimer | VCVAVWRKNDENITLETVCHD |
| | | PKLPYHDFILEDAASPKCIMK |
| | | EKKKPGETFFMCSCSSDECND |
| | | NIIFSEEYNTSNPDIPPHVQK |
| | | SDVEMEAQKDEIICPSCNRTA |
| | | HPLRHINNDMIVTDNNGAVKF |
| | | PQLCKFCDVRFSTCDNQKSCM |
| | | SNCSITSICEKPQEVCVAVWR |
| | | KNDENITLETVCHDPKLPYHD |
| | | FILEDAASPKCIMKEKKKPGE |
| | | TFFMCSCSSDECNDNIIFSEE |
| | | YNTSNPD |
| SEQ ID NO: | TGFβ | LQCFCHLCTKDNFTCVTDGLC |
| 116 | receptor | FVSVTETTDKVIHNSMCIAEI |
| | type I | DLIPRDRPFVCAPSSKTGSVT |
| | ECD | TTYCCNQDHCNKIELPTTVKS |
| | | SPGLGPVEL |

### Tumor-Targeting Moieties

The present disclosure provides, inter alia, multispecific (e.g., bi-, tri-, tetra-specific) molecules, that include, e.g., are engineered to contain, one or two tumor specific targeting moieties, e.g., tumor targeting moieties that bind to MPL and targets other than MPL, that direct the molecule to a tumor cell.

A “tumor-targeting moiety,” as used herein, refers to a binding agent that recognizes or associates with, e.g., binds to, a target in a cancer cell. The tumor-targeting moiety can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the cancer antigen (e.g., MPL, the tumor and/or the stromal antigen). In embodiments, the tumor-targeting moiety specifically binds to the target tumor, e.g., binds preferentially to the target tumor. For example, when the tumor-targeting moiety is an antibody molecule, it binds to the cancer antigen (e.g., MPL, the tumor antigen and/or the stromal antigen) with a dissociation constant of less than about 10 nM, and more typically, 10-100 pM.

In certain embodiments, the multispecific molecules disclosed herein include a tumor-targeting moiety, e.g., a tumor targeting moiety that binds to a target other than MPL. The tumor targeting moiety can be chosen from an antibody molecule (e.g., an antigen binding domain as described herein), a receptor or a receptor fragment, or a ligand or a ligand fragment, or a combination thereof. In some embodiments, the tumor targeting moiety associates with, e.g., binds to, a tumor cell (e.g., a molecule, e.g., antigen, present on the surface of the tumor cell). In certain embodiments, the tumor targeting moiety targets, e.g., directs the multispecific molecules disclosed herein to a cancer (e.g., a cancer or tumor cells). In some embodiments, the cancer is chosen from a hematological cancer, a solid cancer, a metastatic cancer, or a combination thereof.

In some embodiments, the multispecific molecule, e.g., the tumor-targeting moiety, binds to a solid tumor antigen or a stromal antigen. The solid tumor antigen or stromal antigen can be present on a solid tumor, or a metastatic lesion thereof. In some embodiments, the solid tumor is chosen from one or more of pancreatic (e.g., pancreatic adenocarcinoma), breast, colorectal, lung (e.g., small or non-small cell lung cancer), skin, ovarian, or liver cancer. In one embodiment, the solid tumor is a fibrotic or desmoplastic solid tumor. For example, the solid tumor antigen or stromal antigen can be present on a tumor, e.g., a tumor of a class typified by having one or more of: limited tumor perfusion, compressed blood vessels, or fibrotic tumor interstitium. In certain embodiments, the solid tumor antigen is chosen from one or more of: PDL1, CD47, mesothelin, gangloside 2 (GD2), prostate stem cell antigen (PSCA), prostate specific membrane antigen (PMSA), prostate-specific antigen (PSA), carcinoembryonic antigen (CEA), Ron Kinase, c-Met, Immature laminin receptor, TAG-72, BING-4, Calcium-activated chloride channel 2, Cyclin-B1, 9D7, Ep-CAM, EphA3, Her2/neu, Telomerase, SAP-1, Survivin, NY-ESO-1/LAGE-1, PRAME, SSX-2, Melan-A/MART-1, Gp100/pmel17, Tyrosinase, TRP-1/-2, MC1R, β-catenin, BRCA1/2, CDK4, CML66, Fibronectin, p53, Ras, TGF-B receptor, AFP, ETA, MAGE, MUC-1, CA-125, BAGE, GAGE, NY-ESO-1, β-catenin, CDK4, CDCl₂7, CD47, α actinin-4, TRP1/gp75, TRP2, gp100, Melan-A/MART1, gangliosides, WT1, EphA3, Epidermal growth factor receptor (EGFR), CD20, MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, EGFRvIII, gpA33, GD3, GM2, VEGFR, Intergrins (Integrin alphaVbeta3, Integrin alpha5Beta1), Carbohydrates (Le), IGF1R, EPHA3, TRAILR1, TRAILR2, or RANKL.

In some embodiments, the solid tumor antigen is chosen from: PDL1, Mesothelin, CD47, GD2, PMSA, PSCA, CEA, Ron Kinase, or c-Met.

In other embodiments, the multispecific molecule, e.g., the tumor-targeting moiety, binds to a molecule, e.g., antigen, present on the surface of a hematological cancer, e.g., a leukemia or a lymphoma. In some embodiments, the hematological cancer is a B-cell or T cell malignancy. In some embodiments, the hematological cancer is chosen from one or more of a Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), acute myeloid leukemia (AML), chronic myeloid leukemia, myelodysplastic syndrome (MDS), multiple myeloma, or acute lymphocytic leukemia. In embodiments, the cancer is other than acute myeloid leukemia (AML) or myelodysplastic syndrome (MDS). In embodiments, the hematological antigen is chosen from CD19, CD33, CD123, or CD20. In embodiments, the hematological antigen is other than CD33. CD19, In embodiments, the hematological antigen is chosen from CD19, CD20, CD33, CD47, CD123, CD20, CD99, CD30, BCMA, CD38, CD22, SLAMF7, or NY-ESO1.

In some embodiments, any of the multispecific molecules disclosed herein can further include:

(I) a tumor-targeting moiety that comprises:

(a) an antibody molecule against a solid tumor antigen chosen from: Mesothelin, GD2, PMSA, CEA, Ron Kinase, or c-Met; and/or

(b) an antibody molecule against a stromal antigen is chosen from: FAP, hyaluronic acid, collagen IV, tenascin C, or tenascin W; or

(c) a combination of the antibody molecule against the solid tumor antigen and the antibody molecule against the stromal antigen.

In some embodiments, the multifunctional molecule includes a stromal modifying moiety. A “stromal modifying moiety,” as used herein refers to an agent, e.g., a protein (e.g., an enzyme), that is capable of altering, e.g., degrading a component of, the stroma. In embodiments, the component of the stroma is chosen from, e.g., an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin.

### Cytokine Molecules

In some embodiments, the multispecific molecule further includes a cytokine molecule. As used herein, a “cytokine molecule” refers to full length, a fragment or a variant of a cytokine; a cytokine further comprising a receptor domain, e.g., a cytokine receptor dimerizing domain; or an agonist of a cytokine receptor, e.g., an antibody molecule (e.g., an agonistic antibody) to a cytokine receptor, that elicits at least one activity of a naturally-occurring cytokine. In some embodiments the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain. In other embodiments, the cytokine molecule is an agonist of a cytokine receptor, e.g., an antibody molecule (e.g., an agonistic antibody) to a cytokine receptor chosen from an IL-15Ra or IL-21R.

The cytokines are generally polypeptides that influence cellular activity, for example, through signal transduction pathways. Accordingly, a cytokine of the multispecific or multifunctional polypeptide is useful and can be associated with receptor-mediated signaling that transmits a signal from outside the cell membrane to modulate a response within the cell. Cytokines are proteinaceous signaling compounds that are mediators of the immune response. They control many different cellular functions including proliferation, differentiation and cell survival/apoptosis; cytokines are also involved in several pathophysiological processes including viral infections and autoimmune diseases. Cytokines are synthesized under various stimuli by a variety of cells of both the innate (monocytes, macrophages, dendritic cells) and adaptive (T- and B-cells) immune systems. Cytokines can be classified into two groups: pro- and anti-inflammatory. Pro-inflammatory cytokines, including IFNγ, IL-1, IL-6 and TNF-alpha, are predominantly derived from the innate immune cells and Th1 cells. Anti-inflammatory cytokines, including IL-10, IL-4, IL-13 and IL-5, are synthesized from Th2 immune cells.

The present disclosure provides, inter alia, multi-specific (e.g., bi-, tri-, quad-specific) proteins, that include, e.g., are engineered to contain, one or more cytokine molecules, e.g., immunomodulatory (e.g., proinflammatory) cytokines and variants, e.g., functional variants, thereof. Accordingly, in some embodiments, the cytokine molecule is an interleukin or a variant, e.g., a functional variant thereof. In some embodiments the interleukin is a proinflammatory interleukin. In some embodiments the interleukin is chosen from interleukin-2 (IL-2), interleukin-12 (IL-12), interleukin-15 (IL-15), interleukin-18 (IL-18), interleukin-21 (IL-21), interleukin-7 (IL-7), or interferon gamma. In some embodiments, the cytokine molecule is a proinflammatory cytokine.

In certain embodiments, the cytokine is a single chain cytokine. In certain embodiments, the cytokine is a multichain cytokine (e.g., the cytokine comprises 2 or more (e.g., 2) polypeptide chains. An exemplary multichain cytokine is IL-12.

Examples of useful cytokines include, but are not limited to, GM-CSF, IL-1α, IL-1β, IL-2, IL-3, IL-4, IL-5, IL-6, IL-7, IL-8, IL-10, IL-12, IL-21, IFN-α, IFN-β, IFN-γ, MIP-1α, MIP-1β, TGF-β, TNF-α, and TNFβ. In one embodiment the cytokine of the multispecific or multifunctional polypeptide is a cytokine selected from the group of GM-CSF, IL-2, IL-7, IL-8, IL-10, IL-12, IL-15, IL-21, IFN-α, IFN-γ, MIP-1α, MIP-1β and TGF-β.

In one embodiment the cytokine of the multispecific or multifunctional polypeptide is a cytokine selected from the group of IL-2, IL-7, IL-10, IL-12, IL-15, IFN-α, and IFN-γ. In certain embodiments the cytokine is mutated to remove N- and/or O-glycosylation sites. Elimination of glycosylation increases homogeneity of the product obtainable in recombinant production.

In one embodiment, the cytokine of the multispecific or multifunctional polypeptide is IL-2. In a specific embodiment, the IL-2 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity. In another particular embodiment the IL-2 cytokine is a mutant IL-2 cytokine having reduced binding affinity to the .alpha.-subunit of the IL-2 receptor.

The IL-2 or mutant IL-2 cytokine according to any of the above embodiments may comprise additional mutations that provide further advantages such as increased expression or stability. For example, the cysteine at position 125 may be replaced with a neutral amino acid such as alanine, to avoid the formation of disulfide-bridged IL-2 dimers. Thus, in certain embodiments the IL-2 or mutant IL-2 cytokine of the multispecific or multifunctional polypeptide according to the invention comprises an additional amino acid mutation at a position corresponding to residue 125 of human IL-2. In one embodiment said additional amino acid mutation is the amino acid substitution C125A.

In another embodiment, the cytokine of the multispecific or multifunctional polypeptide is IL-15. In a specific embodiment said IL-15 cytokine is a mutant IL-15 cytokine having reduced binding affinity to the α-subunit of the IL-15 receptor. Without wishing to be bound by theory, a mutant IL-15 polypeptide with reduced binding to the .alpha.-subunit of the IL-15 receptor has a reduced ability to bind to fibroblasts throughout the body, resulting in improved pharmacokinetics and toxicity profile, compared to a wild-type IL-15 polypeptide. The use of an cytokine with reduced toxicity, such as the described mutant IL-2 and mutant IL-15 effector moieties, is particularly advantageous in a multispecific or multifunctional polypeptide according to the invention, having a long serum half-life due to the presence of an Fc domain. In one embodiment the mutant IL-15 cytokine of the multispecific or multifunctional polypeptide according to the invention comprises at least one amino acid mutation that reduces or abolishes the affinity of the mutant IL-15 cytokine to the .alpha.-subunit of the IL-15 receptor but preserves the affinity of the mutant IL-15 cytokine to the intermediate-affinity IL-15/IL-2 receptor (consisting of the .beta.- and .gamma.-subunits of the IL-15/IL-2 receptor), compared to the non-mutated IL-15 cytokine. In one embodiment the amino acid mutation is an amino acid substitution. In a specific embodiment, the mutant IL-15 cytokine comprises an amino acid substitution at the position corresponding to residue 53 of human IL-15. In a more specific embodiment, the mutant IL-15 cytokine is human IL-15 comprising the amino acid substitution E53A. In one embodiment the mutant IL-15 cytokine additionally comprises an amino acid mutation at a position corresponding to position 79 of human IL-15, which eliminates the N-glycosylation site of IL-15. Particularly, said additional amino acid mutation is an amino acid substitution replacing an asparagine residue by an alanine residue. In one embodiment, the IL-15 cytokine can elicit one or more of the cellular responses selected from the group consisting of: proliferation in an activated T lymphocyte cell, differentiation in an activated T lymphocyte cell, cytotoxic T cell (CTL) activity, proliferation in an activated B cell, differentiation in an activated B cell, proliferation in a natural killer (NK) cell, differentiation in a NK cell, cytokine secretion by an activated T cell or an NK cell, and NK/lymphocyte activated killer (LAK) antitumor cytotoxicity.

Mutant cytokine molecules useful as effector moieties in the multispecific or multifunctional polypeptide can be prepared by deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing. Substitution or insertion may involve natural as well as non-natural amino acid residues. Amino acid modification includes well known methods of chemical modification such as the addition or removal of glycosylation sites or carbohydrate attachments, and the like.

In one embodiment, the cytokine, particularly a single-chain cytokine, of the multispecific or multifunctional polypeptide is GM-CSF. In a specific embodiment, the GM-CSF cytokine can elicit proliferation and/or differentiation in a granulocyte, a monocyte or a dendritic cell. In one embodiment, the cytokine, particularly a single-chain cytokine, of the multispecific or multifunctional polypeptide is IFN-α. In a specific embodiment, the IFN-α cytokine can elicit one or more of the cellular responses selected from the group consisting of: inhibiting viral replication in a virus-infected cell, and upregulating the expression of major histocompatibility complex I (MHC I). In another specific embodiment, the IFN-α cytokine can inhibit proliferation in a tumor cell. In one embodiment the cytokine, particularly a single-chain cytokine, of the multispecific or multifunctional polypeptide is IFNγ. In a specific embodiment, the IFN-γ cytokine can elicit one or more of the cellular responses selected from the group of: increased macrophage activity, increased expression of MHC molecules, and increased NK cell activity. In one embodiment the cytokine, particularly a single-chain cytokine, of the multispecific or multifunctional polypeptide is IL-7. In a specific embodiment, the IL-7 cytokine can elicit proliferation of T and/or B lymphocytes. In one embodiment, the cytokine, particularly a single-chain cytokine, of the multispecific or multifunctional polypeptide is IL-8. In a specific embodiment, the IL-8 cytokine can elicit chemotaxis in neutrophils. In one embodiment, the cytokine, particularly a single-chain cytokine, of the multispecific or multifunctional polypeptide, is MIP-1α. In a specific embodiment, the MIP-1α cytokine can elicit chemotaxis in monocytes and T lymphocyte cells. In one embodiment, the cytokine, particularly a single-chain cytokine, of the multispecific or multifunctional polypeptide is MIP-1α. In a specific embodiment, the MIP-1α cytokine can elicit chemotaxis in monocytes and T lymphocyte cells. In one embodiment, the cytokine, particularly a single-chain cytokine, of the multispecific or multifunctional polypeptide is TGF-β. In a specific embodiment, the TGF-β cytokine can elicit one or more of the cellular responses selected from the group consisting of: chemotaxis in monocytes, chemotaxis in macrophages, upregulation of IL-1 expression in activated macrophages, and upregulation of IgA expression in activated B cells.

In one embodiment, the multispecific or multifunctional polypeptide of the invention binds to an cytokine receptor with a dissociation constant (K_{D}) that is at least about 1, 1.5, 2, 2.5, 3, 3.5, 4, 4.5, 5, 5.5, 6, 6.5, 7, 7.5, 8, 8.5, 9, 9.5 or 10 times greater than that for a control cytokine. In another embodiment, the multispecific or multifunctional polypeptide binds to an cytokine receptor with a K_{D} that is at least 2, 3, 4, 5, 6, 7, 8, 9, or 10 times greater than that for a corresponding multispecific or multifunctional polypeptide comprising two or more effector moieties. In another embodiment, the multispecific or multifunctional polypeptide binds to an cytokine receptor with a dissociation constant K_{D} that is about 10 times greater than that for a corresponding the multispecific or multifunctional polypeptide comprising two or more cytokines.

In some embodiments, the multispecific molecules disclosed herein include a cytokine molecule. In embodiments, the cytokine molecule includes a full length, a fragment or a variant of a cytokine; a cytokine receptor domain, e.g., a cytokine receptor dimerizing domain; or an agonist of a cytokine receptor, e.g., an antibody molecule (e.g., an agonistic antibody) to a cytokine receptor.

In some embodiments the cytokine molecule is chosen from IL-2, IL-12, IL-15, IL-18, IL-7, IL-21, or interferon gamma, or a fragment or variant thereof, or a combination of any of the aforesaid cytokines. The cytokine molecule can be a monomer or a dimer. In embodiments, the cytokine molecule can further include a cytokine receptor dimerizing domain.

In other embodiments, the cytokine molecule is an agonist of a cytokine receptor, e.g., an antibody molecule (e.g., an agonistic antibody) to a cytokine receptor chosen from an IL-15Ra or IL-21R.

### Immune Cell Engagers

The immune cell engagers of the multispecific molecules disclosed herein can mediate binding to, and/or activation of, an immune cell, e.g., an immune effector cell. In some embodiments, the immune cell is chosen from an NK cell, a B cell, a dendritic cell, or a macrophage cell engager, or a combination thereof. In some embodiments, the immune cell engager is chosen from one, two, three, or all of a a T cell engager, NK cell engager, a B cell engager, a dendritic cell engager, or a macrophage cell engager, or a combination thereof. The immune cell engager can be an agonist of the immune system. In some embodiments, the immune cell engager can be an antibody molecule, a ligand molecule (e.g., a ligand that further comprises an immunoglobulin constant region, e.g., an Fc region), a small molecule, a nucleotide molecule.

“An immune cell engager” refers to one or more binding specificities that bind and/or activate an immune cell, e.g., a cell involved in an immune response. In embodiments, the immune cell is chosen from a T cell, an NK cell, a B cell, a dendritic cell, and/or the macrophage cell. The immune cell engager can be an antibody molecule, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the NK cell antigen, the B cell antigen, the dendritic cell antigen, and/or the macrophage cell antigen). In embodiments, the immune cell engager specifically binds to the target immune cell, e.g., binds preferentially to the target immune cell. For example, when the immune cell engager is an antibody molecule, it binds to the immune cell antigen (e.g., the NK cell antigen, the B cell antigen, the dendritic cell antigen, and/or the macrophage cell antigen) with a dissociation constant of less than about 10 nM, and more typically, 10-100 pM.

### Natural Killer Cell Engagers

Natural Killer (NK) cells recognize and destroy tumors and virus-infected cells in an antibody-independent manner. The regulation of NK cells is mediated by activating and inhibiting receptors on the NK cell surface. One family of activating receptors is the natural cytotoxicity receptors (NCRs) which include NKp30, NKp44 and NKp46. The NCRs initiate tumor targeting by recognition of heparan sulfate on cancer cells. NKG2D is a receptor that provides both stimulatory and costimulatory innate immune responses on activated killer (NK) cells, leading to cytotoxic activity. DNAM1 is a receptor involved in intercellular adhesion, lymphocyte signaling, cytotoxicity and lymphokine secretion mediated by cytotoxic T-lymphocyte (CTL) and NK cell. DAP10 (also known as HCST) is a transmembrane adapter protein which associates with KLRK1 to form an activation receptor KLRK1-HCST in lymphoid and myeloid cells; this receptor plays a major role in triggering cytotoxicity against target cells expressing cell surface ligands such as MHC class I chain-related MICA and MICB, and U(optionally L1)6-binding proteins (ULBPs); it KLRK1-HCST receptor plays a role in immune surveillance against tumors and is required for cytolysis of tumors cells; indeed, melanoma cells that do not express KLRK1 ligands escape from immune surveillance mediated by NK cells. CD16 is a receptor for the Fc region of IgG, which binds complexed or aggregated IgG and also monomeric IgG and thereby mediates antibody-dependent cellular cytotoxicity (ADCC) and other antibody-dependent responses, such as phagocytosis.

In some embodiments, the NK cell engager is a viral hemagglutinin (HA), HA is a glycoprotein found on the surface of influenza viruses. It is responsible for binding the virus to cells with sialic acid on the membranes, such as cells in the upper respiratory tract or erythrocytes. HA has at least 18 different antigens. These subtypes are named H1 through H18. NCRs can recognize viral proteins. NKp46 has been shown to be able to interact with the HA of influenza and the HA-NA of Paramyxovirus, including Sendai virus and Newcastle disease virus. Besides NKp46, NKp44 can also functionally interact with HA of different influenza subtypes.

The present disclosure provides, inter alia, multi-specific (e.g., bi-, tri-, quad-specific) proteins, that are engineered to contain one or more NK cell engager that mediate binding to and/or activation of an NK cell. Accordingly, in some embodiments, the NK cell engager is selected from an antigen binding domain or ligand that binds to (e.g., activates): NKp30, NKp40, NKp44, NKp46, NKG2D, DNAM1, DAP10, DAP12, CD16 (e.g., CD16a, CD16b, or both), CRTAM, CD27, PSGL1, CD96, CD100 (SEMA4D), NKp80, CD244 (also known as SLAMF4 or 2B4), SLAMF6, SLAMF7, KIR2DS2, KIR2DS4, KIR3DS1, KIR2DS3, KIR2DS5, KIR2DS1, CD94, NKG2C, NKG2E, or CD160.

### T Cell Engagers

The present disclosure provides, inter alia, multi-specific (e.g., bi-, tri-, quad-specific) proteins, that are engineered to contain one or more T cell engager that mediate binding to and/or activation of a T cell. Accordingly, in some embodiments, the T cell engager is selected from an antigen binding domain or ligand that binds to (e.g., and in some embodiments activates) one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In other embodiments, the T cell engager is selected from an antigen binding domain or ligand that binds to and does not activate one or more of CD3, TCRα, TCRβ, TCRγ, TCRζ, ICOS, CD28, CD27, HVEM, LIGHT, CD40, 4-1BB, OX40, DR3, GITR, CD30, TIM1, SLAM, CD2, or CD226. In some embodiments, the T cell engager binds to CD3.

### B Cell, Macrophage & Dendritic Cell Engagers

Broadly, B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells (APCs)) and secrete cytokines. Macrophages are a type of white blood cell that engulfs and digests cellular debris, foreign substances, microbes, cancer cells via phagocytosis. Besides phagocytosis, they play important roles in nonspecific defense (innate immunity) and also help initiate specific defense mechanisms (adaptive immunity) by recruiting other immune cells such as lymphocytes. For example, they are important as antigen presenters to T cells. Beyond increasing inflammation and stimulating the immune system, macrophages also play an important anti-inflammatory role and can decrease immune reactions through the release of cytokines. Dendritic cells (DCs) are antigen-presenting cells that function in processing antigen material and present it on the cell surface to the T cells of the immune system.

The present disclosure provides, inter alia, multi-specific (e.g., bi-, tri-, quad-specific) proteins, that include, e.g., are engineered to contain, one or more B cell, macrophage, and/or dendritic cell engager that mediate binding to and/or activation of a B cell, macrophage, and/or dendritic cell.

Accordingly, in some embodiments, the immune cell engager comprises a B cell, macrophage, and/or dendritic cell engager chosen from one or more of CD40 ligand (CD40L) or a CD70 ligand; an antibody molecule that binds to CD40 or CD70; an antibody molecule to OX40; an OX40 ligand (OX40L); an agonist of a Toll-like receptor (e.g., as described herein, e.g., a TLR4, e.g., a constitutively active TLR4 (caTLR4), or a TLR9 agonists); a 41BB; a CD2; a CD47; or a STING agonist, or a combination thereof.

In some embodiments, the B cell engager is a CD40L, an OX40L, or a CD70 ligand, or an antibody molecule that binds to OX40, CD40 or CD70.

In some embodiments, the macrophage engager is a CD2 agonist. In some embodiments, the macrophage engager is an antigen binding domain that binds to: CD40L or antigen binding domain or ligand that binds CD40, a Toll like receptor (TLR) agonist (e.g., as described herein), e.g., a TLR9 or TLR4 (e.g., caTLR4 (constitutively active TLR4), CD47, or a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In some embodiments, the dendritic cell engager is a CD2 agonist. In some embodiments, the dendritic cell engager is a ligand, a receptor agonist, or an antibody molecule that binds to one or more of: OX40L, 41BB, a TLR agonist (e.g., as described herein) (e.g., TLR9 agonist, TLR4 (e.g., caTLR4 (constitutively active TLR4)), CD47, or and a STING agonist. In some embodiments, the STING agonist is a cyclic dinucleotide, e.g., cyclic di-GMP (cdGMP) or cyclic di-AMP (cdAMP). In some embodiments, the STING agonist is biotinylated.

In other embodiments, the immune cell engager mediates binding to, or activation of, one or more of a B cell, a macrophage, and/or a dendritic cell. Exemplary B cell, macrophage, and/or dendritic cell engagers can be chosen from one or more of CD40 ligand (CD40L) or a CD70 ligand; an antibody molecule that binds to CD40 or CD70; an antibody molecule to OX40; an OX40 ligand (OX40L); a Toll-like receptor agonist (e.g., a TLR4, e.g., a constitutively active TLR4 (caTLR4) or a TLR9 agonist); a 41BB agonist; a CD2; a CD47; or a STING agonist, or a combination thereof.

In some embodiments, the B cell engager is chosen from one or more of a CD40L, an OX40L, or a CD70 ligand, or an antibody molecule that binds to OX40, CD40 or CD70.

In other embodiments, the macrophage cell engager is chosen from one or more of a CD2 agonist; a CD40L; an OX40L; an antibody molecule that binds to OX40, CD40 or CD70; a Toll-like receptor agonist or a fragment thereof (e.g., a TLR4, e.g., a constitutively active TLR4 (caTLR4)); a CD47 agonist; or a STING agonist.

In other embodiments, the dendritic cell engager is chosen from one or more of a CD2 agonist, an OX40 antibody, an OX40L, 41BB agonist, a Toll-like receptor agonist or a fragment thereof (e.g., a TLR4, e.g., a constitutively active TLR4 (caTLR4)), CD47 agonist, or a STING agonist.

In yet other embodiments, the STING agonist comprises a cyclic dinucleotide, e.g., a cyclic di-GMP (cdGMP), a cyclic di-AMP (cdAMP), or a combination thereof, optionally with 2′,5′ or 3′,5′ phosphate linkages.

### Toll-Like Receptors

Toll-Like Receptors (TLRs) are evolutionarily conserved receptors are homologues of the Drosophila Toll protein, and recognize highly conserved structural motifs known as pathogen-associated microbial patterns (PAMPs), which are exclusively expressed by microbial pathogens, or danger-associated molecular patterns (DAMPs) that are endogenous molecules released from necrotic or dying cells. PAMPs include various bacterial cell wall components such as lipopolysaccharide (LPS), peptidoglycan (PGN) and lipopeptides, as well as flagellin, bacterial DNA and viral double-stranded RNA. DAMPs include intracellular proteins such as heat shock proteins as well as protein fragments from the extracellular matrix. Stimulation of TLRs by the corresponding PAMPs or DAMPs initiates signaling cascades leading to the activation of transcription factors, such as AP-1, NF-κB and interferon regulatory factors (IRFs). Signaling by TLRs results in a variety of cellular responses, including the production of interferons (IFNs), pro-inflammatory cytokines and effector cytokines that direct the adaptive immune response. TLRs are implicated in a number of inflammatory and immune disorders and play a role in cancer (Rakoff-Nahoum S. & Medzhitov R., 2009. Toll-like receptors and cancer. Nat Revs Cancer 9:57-63.)

TLRs are type I transmembrane proteins characterized by an extracellular domain containing leucine-rich repeats (LRRs) and a cytoplasmic tail that contains a conserved region called the Toll/IL-1 receptor (TIR) domain. Ten human and twelve murine TLRs have been characterized, TLR1 to TLR10 in humans, and TLR1 to TLR9, TLR11, TLR12 and TLR13 in mice, the homolog of TLR10 being a pseudogene. TLR2 is essential for the recognition of a variety of PAMPs from Gram-positive bacteria, including bacterial lipoproteins, lipomannans and lipoteichoic acids. TLR3 is implicated in virus-derived double-stranded RNA. TLR4 is predominantly activated by lipopolysaccharide. TLR5 detects bacterial flagellin and TLR9 is required for response to unmethylated CpG DNA. Finally, TLR7 and TLR8 recognize small synthetic antiviral molecules, and single-stranded RNA was reported to be their natural ligand. TLR11 has been reported to recognize uropathogenic E. coli and a profilin-like protein from Toxoplasma gondii. The repertoire of specificities of the TLRs is apparently extended by the ability of TLRs to heterodimerize with one another. For example, dimers of TLR2 and TLR6 are required for responses to diacylated lipoproteins while TLR2 and TLR1 interact to recognize triacylated lipoproteins. Specificities of the TLRs are also influenced by various adapter and accessory molecules, such as MD-2 and CD14 that form a complex with TLR4 in response to LPS.

TLR signaling consists of at least two distinct pathways: a MyD88-dependent pathway that leads to the production of inflammatory cytokines, and a MyD88-independent pathway associated with the stimulation of IFN-β and the maturation of dendritic cells. The MyD88-dependent pathway is common to all TLRs, except TLR3 (Adachi O. et al., 1998. Targeted disruption of the MyD88 gene results in loss of IL-1- and IL-18-mediated function. Immunity. 9(1):143-50). Upon activation by PAMPs or DAMPs, TLRs hetero- or homodimerize inducing the recruitment of adaptor proteins via the cytoplasmic TIR domain. Individual TLRs induce different signaling responses by usage of the different adaptor molecules. TLR4 and TLR2 signaling requires the adaptor TIRAP/Mal, which is involved in the MyD88-dependent pathway. TLR3 triggers the production of IFN-β in response to double-stranded RNA, in a MyD88-independent manner, through the adaptor TRIF/TICAM-1. TRAM/TICAM-2 is another adaptor molecule involved in the MyD88-independent pathway which function is restricted to the TLR4 pathway.

TLR3, TLR7, TLR8 and TLR9 recognize viral nucleic acids and induce type I IFNs. The signaling mechanisms leading to the induction of type I IFNs differ depending on the TLR activated. They involve the interferon regulatory factors, IRFs, a family of transcription factors known to play a critical role in antiviral defense, cell growth and immune regulation. Three IRFs (IRF3, IRF5 and IRF7) function as direct transducers of virus-mediated TLR signaling. TLR3 and TLR4 activate IRF3 and IRF7, while TLR7 and TLR8 activate IRF5 and IRF7 (Doyle S. et al., 2002. IRF3 mediates a TLR3/TLR4-specific antiviral gene program. Immunity. 17(3):251-63). Furthermore, type I IFN production stimulated by TLR9 ligand CpG-A has been shown to be mediated by PI(3)K and mTOR (Costa-Mattioli M. & Sonenberg N. 2008. RAPping production of type I interferon in pDCs through mTOR. Nature Immunol. 9: 1097-1099).

TLR-9

TLR9 recognizes unmethylated CpG sequences in DNA molecules. CpG sites are relatively rare (˜1%) on vertebrate genomes in comparison to bacterial genomes or viral DNA. TLR9 is expressed by numerous cells of the immune system such as B lymphocytes, monocytes, natural killer (NK) cells, and plasmacytoid dendritic cells. TLR9 is expressed intracellularly, within the endosomal compartments and functions to alert the immune system of viral and bacterial infections by binding to DNA rich in CpG motifs. TLR9 signals leads to activation of the cells initiating pro-inflammatory reactions that result in the production of cytokines such as type-I interferon and IL-12.

### TLR Agonists

A TLR agonist can agonize one or more TLR, e.g., one or more of human TLR-1, 2, 3, 4, 5, 6, 7, 8, 9, or 10. In some embodiments, an adjunctive agent described herein is a TLR agonist. In some embodiments, the TLR agonist specifically agonizes human TLR-9. In some embodiments, the TLR-9 agonist is a CpG moiety. As used herein, a CpG moiety, is a linear dinucleotide having the sequence: 5′-C-phosphate-G-3′, that is, cytosine and guanine separated by only one phosphate.

In some embodiments, the CpG moiety comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more CpG dinucleotides. In some embodiments, the CpG moiety consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 CpG dinucleotides. In some embodiments, the CpG moiety has 1-5, 1-10, 1-20, 1-30, 1-40, 1-50, 5-10, 5-20, 5-30, 10-20, 10-30, 10-40, or 10-50 CpG dinucleotides.

In some embodiments, the TLR-9 agonist is a synthetic ODN (oligodeoxynucleotides). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. There are three major classes of CpG ODNs: classes A, B and C, which differ in their immunostimulatory activities. CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

### Exemplary Multispecific Molecules

The disclosure relates, inter alia, to novel multispecific molecules that include a first MPL-targeting moiety; and none, one, two or three of:

(i) a second MPL targeting moiety, e.g., a second MPL targeting moiety whose binding site does not overlap with the binding site of the first MPL targeting moiety;

(ii) an immune cell engager (e.g., chosen from one, two, three, or all of an NK cell engager, a B cell engager, a dendritic cell engager, or a macrophage cell engager);

(iii) a cytokine molecule; or

(iv) a tumor targeting molecule, e.g., a tumor targeting molecule against a target other than MPL.

Without being bound by theory, the multispecific molecules disclosed herein are expected to target (e.g., localize, bridge and/or activate) an immune cell (e.g., an immune effector cell chosen form an NK cell, a B cell, a dendritic cell or a macrophage), at a cancer cell. Increasing the proximity and/or activity of the immune cell using the multispecific molecules described herein is expected to enhance an immune response against the cancer cell, thereby providing a more effective cancer therapy. Accordingly, provided herein are, inter alia, multispecific molecules (e.g., multispecific antibody molecules) that include the aforesaid moieties, nucleic acids encoding the same, methods of producing the aforesaid molecules, and methods of treating a cancer using the aforesaid molecules.

In some embodiments, the multispecific molecule includes a single chain antibody molecule, e.g., a single domain antibody, a scFv, a camelid, or a shark antibody, and a second moiety. In some embodiments, the multispecific molecule comprises a VH to VL from N to C orientation, of the scFv connected, optionally via a linker, to the second moiety; the scFv can form the first binding specificity. In some embodiments, the second moiety is located before the VH region of the scFv from an N- to C-orientation, or after the VL region of the scFv from an N- to C-orientation; the second moiety can form the second binding specificity. In other embodiments, the multispecific molecule comprises a VL to VH from N to C orientation, of the scFv connected, optionally via a linker, to the second moiety; the scFv can form the first binding specificity. In some embodiments, the second moiety is located before the VL region of the scFv from an N- to C-orientation, or after the VH region of the scFv from an N- to C-orientation; the second moiety can form the second binding specificity. In embodiments, the scFv can be a tumor targeting moiety (e.g., binds to a cancer antigen, e.g., a solid tumor, stromal, or hematological antigen), or can be an immune cell engager (e.g., binds to an immune cell antigen). In other embodiments, the second moiety is a tumor targeting moiety (e.g., in embodiments where the scFv is not the tumor targeting moiety), an immune cell engager (e.g., in embodiments where the scFv is not the immune cell engager), or a cytokine molecule (e.g., as described herein). In embodiments, partner A can be an antibody molecule (e.g., a single chain antibody molecule (e.g., a scFv) or a Fab), a receptor molecule, a ligand molecule (e.g., a receptor ligand or a cytokine molecule), e.g., as described herein. In one embodiment, the tumor-targeting moiety is a scFv to a cancer cell antigen, and the second moiety is chosen from a cytokine molecule or an immune cell engager. In some embodiments, the second moiety is a second antibody molecule (e.g., a second scFv or Fab), a receptor molecule, a ligand molecule (e.g., a receptor ligand or a cytokine molecule).

In some embodiments, the multispecific molecule includes a single chain antibody molecule, e.g., a single domain antibody, a scFv, a camelid, or a shark antibody, and a second moiety. In some embodiments, the multispecific molecule comprises a VH to VL from N to C orientation, of the scFv connected, optionally via a linker, to a second moiety and/or a third moiety; the scFv can form the first binding specificity. In some embodiments, the second or third moieties is located before the VH region of the scFv from an N- to C-orientation and the third moiety after the VL region of the scFv from an N- to C-orientation, respectively; the second and third moieties can form the second and third binding specificities. In other embodiments, the multispecific molecule comprises a VL to VH from N to C orientation, of the scFv connected, optionally via a linker, to a second moiety and/or a third moiety. In some embodiments, the second moiety is located before the VL region of the scFv from an N- to C-orientation, and the third moiety after the VH region of the scFv from an N- to C-orientation; the second and third moieties can form the second and third binding specificities. In embodiments, the scFv of any of the aforesaid multispecific molecules can be a tumor targeting moiety (e.g., bind to a cancer antigen, e.g., a solid tumor, stromal or hematological antigen) or can be an immune cell engager (e.g., bind to an immune cell antigen). In embodiments, the second moiety and third moiety is independently chosen from a tumor targeting moiety, an immune cell engager, or a cytokine molecule (e.g., as described herein). In embodiments, partner A and/or partner B can be an antibody molecule (e.g., a single chain antibody molecule (e.g., a scFv or a Fab), a receptor molecule, or a ligand molecule (e.g., a receptor ligand or a cytokine molecule), e.g., as described herein. In one embodiment, the tumor-targeting moiety is a scFv to a cancer cell antigen, and the second moiety and third moiety is independently chosen from a cytokine molecule or an immune cell engager. In some embodiments, the second and third moiety is independently chosen from a second antibody molecule (e.g., a second scFv or Fab), a receptor molecule, or a ligand molecule (e.g., a receptor ligand or a cytokine molecule).

In some embodiments, the multispecific molecule does not consist of a single chain polypeptide of an NK cell engager (i.e., a scFv) that binds to CD16 (Fc□RIII), and a tumor targeting moiety, i.e., a scFv targeting CD33. In other embodiments, the multispecific molecule does not consist of a single chain polypeptide of the scFv that binds to CD16, an IL-15 cytokine, and the scFv targeting CD33.

In embodiments, the multispecific molecule is a bispecific or bifunctional molecule, wherein the first and second polypeptides (i) and (ii) are non-contiguous, e.g., are two separate polypeptide chains.

In embodiments, the second moiety, can be an antibody molecule (e.g., a single chain antibody molecule (e.g., a scFv) or a Fab), a receptor molecule, a ligand molecule (e.g., a receptor ligand or a cytokine molecule), e.g., as described herein. In one embodiment, the multispecific molecule includes a Fab molecule and the second moiety is chosen from a second antibody molecule (e.g., a scFv or a second Fab), a receptor molecule, or a receptor ligand molecule, or a cytokine molecule. In one embodiment, the tumor-targeting moiety is a Fab to a cancer cell antigen, and the second moiety is chosen from a cytokine molecule or an immune cell engager. In some embodiments, the second moiety is a second antibody molecule (e.g., a second scFv or Fab), a receptor molecule, or a receptor ligand molecule, or a cytokine molecule.

In embodiments, the multispecific molecule is a bispecific or bifunctional molecule, wherein the first and second polypeptides (i) and (ii) are non-contiguous, e.g., are two separate polypeptide chains. In embodiments, the second moiety can be an antibody molecule (e.g., a single chain antibody molecule (e.g., a scFv) or a Fab), a receptor molecule, or a ligand molecule (e.g., a receptor ligand or a cytokine molecule), e.g., as described herein. In one embodiment, the multispecific molecule includes a Fab molecule and the second moiety is chosen from a second antibody molecule (e.g., a scFv or a second Fab), a receptor molecule, or a ligand molecule (e.g., a cytokine molecule). In one embodiment, the tumor-targeting moiety is a Fab to a cancer cell antigen, and the second moiety is chosen from a cytokine molecule or an immune cell engager. In some embodiments, the second moiety is a second antibody molecule (e.g., a second scFv or Fab), a receptor molecule, a receptor ligand molecule, or a cytokine molecule.

In one embodiment, the multispecific molecule includes at least two or at least three or at least four non-contiguous polypeptides, wherein:

(i) the first polypeptide includes from N- to C-orientation a first immunoglobulin constant region (e.g., a CH2 connected to a CH3 region) (e.g., a first Fc region); and

(ii) the second polypeptide includes from N- to C-orientation a second immunoglobulin constant region (e.g., a CH2 connected to a CH3 region) (e.g., a second Fc region).

In embodiments, the multispecific molecule is a bispecific or bifunctional molecule, wherein the first and second polypeptides (i) and (ii) are non-contiguous, e.g., are two separate polypeptide chains. In some embodiments, the first and second polypeptides (i) and (ii) include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the first immunoglobulin chain constant region (e.g., the first Fc region) can include an amino acid substitution chosen from: T366S, L368A, or Y407V (e.g., corresponding to a cavity or hole), and the second immunoglobulin chain constant region (e.g., the second Fc region) includes a T366W (e.g., corresponding to a protuberance or knob). In some embodiments, the first and second polypeptides are a first and second member of a heterodimeric first and second Fc region.

In embodiments, the first and second binding specificities is each independently chosen from an antibody molecule (e.g., a single chain antibody molecule (e.g., a scFv) or a Fab), a receptor molecule, a ligand molecule (e.g., a receptor ligand or a cytokine molecule), e.g., as described herein. In embodiments, the first and second binding specificities are connected to either the first or the second polypeptide, or each of the polypeptides (e.g., one or both members of a heterodimeric Fc molecule). In one embodiment, the first binding specificity is connected to the N-terminal end of the first polypeptide (e.g., a —CH2-CH3-region of the first Fc molecule), and the second binding specificity is connected to the N-terminal end of the second polypeptide (e.g., a —CH2-CH3-region of the second Fc molecule). Alternatively, the first binding specificity (e.g., partner A) is connected to the C-terminal end of the first polypeptide (e.g., a —CH2-CH3-region of the first Fc molecule), and the second binding specificity is connected to the C-terminal end of the second polypeptide (e.g., a —CH2-CH3-region of the second Fc molecule). Alternatively, the first binding specificity is connected to the N-terminal end of the first polypeptide (e.g., a —CH2-CH3-region of the first Fc molecule), and the second binding specificity (e.g., partner B) is connected to the C-terminal end of the second polypeptide (e.g., a —CH2-CH3-region of the second Fc molecule). In other embodiments, the second binding specificity is connected to N-terminus of the first polypeptide (e.g., the —CH2-CH3-region of the first Fc molecule), and the first binding specificity is connected to the C-terminal end of the second polypeptide (e.g., a —CH2-CH3-region of the second Fc molecule). In one embodiment, the first —CH2-CH3 region includes a protuberance or knob, and the second —CH2-CH3 region includes a cavity or hole).

In some embodiments, the first and second binding specificities of the bispecific molecule can each be independently chosen from a tumor targeting moiety, a cytokine molecule, a T cell engager, an NK cell engager, a B cell engager, a dendritic cell engager, or a macrophage cell engager. In some embodiments, the first binding specificity is a tumor targeting moiety and the second binding specificity is chosen from a cytokine molecule, an NK cell engager, a T cell engager, a B cell engager, a dendritic cell engager, or a macrophage cell engager.

In some embodiments, the first binding specificity is a tumor targeting moiety and the second binding specificity is chosen from a cytokine molecule, an NK cell engager, a T cell engager, a B cell engager, a dendritic cell engager, or a macrophage cell engager.

In one embodiment, the multispecific molecule is a bispecific molecule that includes two non-contiguous first and second polypeptides. In embodiments, the first and second polypeptides, include, respectively, a first and a second binding sites, which are independently chosen from an antibody molecule (e.g., a single chain antibody molecule (e.g., a scFv) or a Fab), a receptor molecule, a ligand molecule (e.g., a receptor ligand, or a cytokine molecule), e.g., as described herein. In some embodiments, the first and second binding specificities are each independently chosen from MPL, a tumor targeting moiety, a cytokine molecule, an NK cell engager, a T cell engager, a B cell engager, a dendritic cell engager, or a macrophage cell engager, e.g., as described herein.

In another embodiment, the multispecific molecule is a bispecific molecule that includes two or at least three non-contiguous first and second polypeptides, wherein:

(i) the first polypeptide includes from N- to C-orientation a first binding specificity, e.g., a first antibody molecule, connected, optionally via a linker, to a first immunoglobulin constant region (e.g., a CH2 connected to a CH3 region) (e.g., a first Fc region);

(ii) the second polypeptide includes from N- to C-orientation a second immunoglobulin constant region (e.g., a CH2 connected to a CH3 region) (e.g., a second Fc region); and

(optionally) (iii) a third polypeptide comprising a portion of the first antibody molecule or a second antibody molecule.

In embodiments, the first and second polypeptides, include, respectively, a first and a second binding specificities (e.g., sites), which are independently chosen from an antibody molecule (e.g., a single chain antibody molecule (e.g., a scFv) or a Fab), a receptor molecule, a ligand molecule (e.g., a receptor ligand, or a cytokine molecule), e.g., as described herein. In some embodiments, the first and second binding specificities are each independently chosen from a tumor targeting moiety, a cytokine molecule, an NK cell engager, a T cell engager, a B cell engager, a dendritic cell engager, or a macrophage cell engager, e.g., as described herein.

In some embodiments, the first polypeptide has the following configuration from N-to-C:

(a) a first portion of a first antigen domain, e.g., a first VH-CH1 of a Fab molecule, that binds to, e.g., a cancer antigen, e.g., a solid tumor, stromal or hematological antigen (e.g., MPL), connected, optionally, via a linker to, the first immunoglobulin constant region (e.g., the CH2 connected to the CH3 region) (e.g., a first Fc region);

(b) a second binding specificity (e.g., a second binding site), which is chosen from a cytokine molecule, or an immune cell engager, connected, optionally, via a linker to, the second immunoglobulin constant region (e.g., the CH2 connected to the CH3 region) (e.g., the second Fc region); and

(c) the third polypeptide has the following configuration from N-to-C: a second portion of the first antigen domain, e.g., a first VL-CL of the Fab, that binds to, e.g., a cancer antigen, e.g., a solid tumor, stromal or hematological antigen (e.g., the same cancer antigen bound by the first VH-CH1)

In embodiments, the first immunoglobulin constant region (e.g., the first CH2-CH3 region) includes a protuberance or knob, e.g., as described herein.

In embodiments, the second immunoglobulin constant region (e.g., the second CH2-CH3 region) includes a cavity or hole. In embodiments, the first and second immunoglobulin constant region promote heterodimerization of the bispecific molecule.

In other embodiments, the multispecific molecule includes a first, a second and a third non-contiguous polypeptide, wherein:

n MPL-targeting moiety, e.g., an antibody molecule (e.g., a first portion of a first antigen domain, e.g., a first VH-CH1 of a Fab molecule), that binds to, e.g., an MPL antigen, connected, optionally, via a linker to, a first domain that promotes association between the first and the second polypeptide (e.g., a first immunoglobulin constant domain (e.g., a first Fc molecule as described herein);

(ii) the second polypeptide includes, e.g., in the N- to C-orientation, a cytokine molecule or an immune cell engager (e.g., an antibody molecule, e.g., a scFv, that binds to an immune cell antigen), connected, optionally, via a linker to, a second domain that promotes association between the first and the second polypeptide (e.g., a second immunoglobulin constant domain (e.g., a second Fc molecule as described herein); and

(iii) the third polypeptide includes, e.g., in the N- to C-orientation, a second portion of the first antigen domain, e.g., a first VL-CL of the Fab, that binds to, e.g., an MPLantigen (e.g., the same MPL antigen bound by the first VH-CH1).

In some embodiments, the multispecific molecule includes a Fab molecule targeting-MPL connected, optionally, via a linker to, a first Fc molecule; a cytokine or immune cell engager (e.g., a scFv), connected, optionally, via a linker to, a second Fc molecule. In embodiments, the multispecific molecule is a bispecific molecule.

In embodiments, the MPL targeting moiety of the first polypeptide comprises a light chain variable domain of a tumor targeting molecule (e.g., Fab); and the tumor targeting moiety of the second polypeptide comprises a heavy chain variable domain of a MPL targeting molecule (e.g., Fab).

In other embodiments, the first MPL targeting moiety of the first polypeptide comprises a heavy chain variable domain of an MPL targeting molecule (e.g., Fab); and the second MPL targeting moiety of the second polypeptide comprises a light chain variable domain of an MPL targeting molecule (e.g., Fab).

In other embodiments, the first MPL targeting moiety of the first polypeptide comprises a light chain variable domain of an MPL targeting molecule (e.g., Fab); and the second MPL targeting moiety of the second polypeptide comprises a heavy chain variable domain of an MPL targeting molecule (e.g., Fab).

In other embodiments, the MPL targeting moiety of the first polypeptide comprises an MPL targeting scFv; and the MPL targeting moiety of the second polypeptide comprises an MPL targeting scFv.

Linkers

The multispecific molecule disclosed herein can further include a linker, e.g., a linker between one or more of: the targeting moiety and the cytokine molecule, the targeting moiety and the immune cell engager, the cytokine molecule and the immune cell engager, the cytokine molecule and the immunoglobulin chain constant region (e.g., the Fc region), the targeting moiety and the immunoglobulin chain constant region, or the immune cell engager and the immunoglobulin chain constant region. In embodiments, the linker chosen from: a cleavable linker, a non-cleavable linker, a peptide linker, a flexible linker, a rigid linker, a helical linker, or a non-helical linker, or a combination thereof.

In one embodiment, the multispecific molecule can include one, two, three or four linkers, e.g., a peptide linker. In one embodiment, the peptide linker includes Gly and Ser, e.g., a peptide linker chosen from: GGGGS (SEQ ID NO: 117); GGGGSGGGGS (SEQ ID NO: 118); GGGGSGGGGSGGGGS (SEQ ID NO: 119); or DVPSGPGGGGGSGGGGS (SEQ ID NO: 120).

### Antibody Molecules

In one embodiment, the antibody molecule binds to a cancer antigen, e.g., a tumor antigen. In some embodiments, the cancer antigen is, e.g., a mammalian, e.g., a human, cancer antigen. In other embodiments, the antibody molecule binds to an immune cell antigen, e.g., a mammalian, e.g., a human, immune cell antigen. For example, the antibody molecule binds specifically to an epitope, e.g., linear or conformational epitope, on the cancer antigen or the immune cell antigen.

In an embodiment, an antibody molecule is a monospecific antibody molecule and binds a single epitope. E.g., a monospecific antibody molecule having a plurality of immunoglobulin variable domain sequences, each of which binds the same epitope.

In an embodiment an antibody molecule is a multispecific antibody molecule, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a multispecific antibody molecule comprises a third, fourth or fifth immunoglobulin variable domain. In an embodiment, a multispecific antibody molecule is a bispecific antibody molecule, a trispecific antibody molecule, or a tetraspecific antibody molecule.

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., the different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv or a Fab, or fragment thereof, have binding specificity for a first epitope and a scFv or a Fab, or fragment thereof, have binding specificity for a second epitope.

In an embodiment, an antibody molecule comprises a diabody, and a single-chain molecule, as well as an antigen-binding fragment of an antibody (e.g., Fab, F(ab′)2, and Fv). For example, an antibody molecule can include a heavy (H) chain variable domain sequence (abbreviated herein as VH), and a light (L) chain variable domain sequence (abbreviated herein as VL). In an embodiment an antibody molecule comprises or consists of a heavy chain and a light chain (referred to herein as a half antibody. In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The a preparation of antibody molecules can be monoclonal or polyclonal. An antibody molecule can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda. The term “immunoglobulin” (Ig) is used interchangeably with the term “antibody” herein.

Examples of antigen-binding fragments of an antibody molecule include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments are obtained using conventional techniques known to those with skill in the art, and the fragments are screened for utility in the same manner as are intact antibodies.

Antibody molecules include intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function).

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

Each VH and VL typically includes three CDRs and four FRs, arranged from amino-terminus to carboxy-terminus in the following order: FR1, CDR1, FR2, CDR2, FR3, CDR3, FR4.

The antibody molecule can be a polyclonal or a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

The antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. Preferably, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody molecule can be one in which the variable region, or a portion thereof, e.g., the CDRs, are generated in a non-human organism, e.g., a rat or mouse. Chimeric, CDR-grafted, and humanized antibodies are within the invention. Antibody molecules generated in a non-human organism, e.g., a rat or mouse, and then modified, e.g., in the variable framework or constant region, to decrease antigenicity in a human are within the invention.

An “effectively human” protein is a protein that does substantially not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding to the antigen. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody molecule can be humanized by methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibody molecules can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibody molecules in which specific amino acids have been substituted, deleted or added. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

The antibody molecule can be a single chain antibody. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has: effector function; and can fix complement. In other embodiments the antibody does not; recruit effector cells; or fix complement. In another embodiment, the antibody has reduced or no ability to bind an Fc receptor. For example, it is a isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules of the invention are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody molecule is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

### Multispecific Antibody Molecules

Exemplary structures of multispecific and multifunctional molecules defined herein are described throughout. Exemplary structures are further described in: Weidle U et al. (2013) The Intriguing Options of Multispecific Antibody Formats for Treatment of Cancer. Cancer Genomics & Proteomics 10: 1-18 (2013); and Spiess C et al. (2015) Alternative molecular formats and therapeutic applications for bispecific antibodies. Molecular Immunology 67: 95-106; the full contents of each of which is incorporated by reference herein).

In embodiments, multispecific antibody molecules can comprise more than one antigen-binding site, where different sites are specific for different antigens. In embodiments, multispecific antibody molecules can bind more than one (e.g., two or more) epitopes on the same antigen. In embodiments, multispecific antibody molecules comprise an antigen-binding site specific for a target cell (e.g., cancer cell) and a different antigen-binding site specific for an immune effector cell. In one embodiment, the multispecific antibody molecule is a bispecific antibody molecule. Bispecific antibody molecules can be classified into five different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates.

BsIgG is a format that is monovalent for each antigen. Exemplary BsIgG formats include but are not limited to crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, kλ-body, orthogonal Fab. See Spiess et al. Mol. Immunol. 67(2015):95-106. Exemplary BsIgGs include catumaxomab (Fresenius Biotech, Trion Pharma, Neopharm), which contains an anti-CD3 arm and an anti-EpCAM arm; and ertumaxomab (Neovii Biotech, Fresenius Biotech), which targets CD3 and HER2. In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. See Spiess et al. Mol. Immunol. 67(2015):95-106. Strategies that have been used to avoid heavy chain pairing of homodimers in BsIgG include knobs-in-holes, duobody, azymetric, charge pair, HA-TF, SEEDbody, and differential protein A affinity. See Id. BsIgG can be produced by separate expression of the component antibodies in different host cells and subsequent purification/assembly into a BsIgG. BsIgG can also be produced by expression of the component antibodies in a single host cell. BsIgG can be purified using affinity chromatography, e.g., using protein A and sequential pH elution.

IgG appended with an additional antigen-binding moiety is another format of bispecific antibody molecules. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). See Id. Examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. An example of an IgG-scFv is MM-141 (Merrimack Pharmaceuticals), which binds IGF-1R and HERS. Examples of DVD-Ig include ABT-981 (AbbVie), which binds IL-1α and IL-1β; and ABT-122 (AbbVie), which binds TNF and IL-17A.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Exemplary bispecific antibody fragments include but are not limited to nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody. See Id. For example, the BiTE format comprises tandem scFvs, where the component scFvs bind to CD3 on T cells and a surface antigen on cancer cells

Bispecific fusion proteins include antibody fragments linked to other proteins, e.g., to add additional specificity and/or functionality. An example of a bispecific fusion protein is an immTAC, which comprises an anti-CD3 scFv linked to an affinity-matured T-cell receptor that recognizes HLA-presented peptides. In embodiments, the dock-and-lock (DNL) method can be used to generate bispecific antibody molecules with higher valency. Also, fusions to albumin binding proteins or human serum albumin can be extend the serum half-life of antibody fragments. See Id.

In embodiments, chemical conjugation, e.g., chemical conjugation of antibodies and/or antibody fragments, can be used to create BsAb molecules. See Id. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life of the low molecular weight drug. An exemplary CovX-body is CVX-241 (NCT01004822), which comprises an antibody conjugated to two short peptides inhibiting either VEGF or Ang2. See Id.

The antibody molecules can be produced by recombinant expression, e.g., of at least one or more component, in a host system. Exemplary host systems include eukaryotic cells (e.g., mammalian cells, e.g., CHO cells, or insect cells, e.g., SF9 or S2 cells) and prokaryotic cells (e.g., E. coli). Bispecific antibody molecules can be produced by separate expression of the components in different host cells and subsequent purification/assembly. Alternatively, the antibody molecules can be produced by expression of the components in a single host cell. Purification of bispecific antibody molecules can be performed by various methods such as affinity chromatography, e.g., using protein A and sequential pH elution. In other embodiments, affinity tags can be used for purification, e.g., histidine-containing tag, myc tag, or streptavidin tag.

### CDR-Grafted Scaffolds

In embodiments, the antibody molecule is a CDR-grafted scaffold domain. In embodiments, the scaffold domain is based on a fibronectin domain, e.g., fibronectin type III domain. The overall fold of the fibronectin type III (Fn3) domain is closely related to that of the smallest functional antibody fragment, the variable domain of the antibody heavy chain. There are three loops at the end of Fn3; the positions of BC, DE and FG loops approximately correspond to those of CDR1, 2 and 3 of the VH domain of an antibody. Fn3 does not have disulfide bonds; and therefore Fn3 is stable under reducing conditions, unlike antibodies and their fragments (see, e.g., WO 98/56915; WO 01/64942; WO 00/34784). An Fn3 domain can be modified (e.g., using CDRs or hypervariable loops described herein) or varied, e.g., to select domains that bind to an antigen/marker/cell described herein.

In embodiments, a scaffold domain, e.g., a folded domain, is based on an antibody, e.g., a “minibody” scaffold created by deleting three beta strands from a heavy chain variable domain of a monoclonal antibody (see, e.g., Tramontano et al., 1994, J Mol. Recognit. 7:9; and Martin et al., 1994, EMBO J. 13:5303-5309). The “minibody” can be used to present two hypervariable loops. In embodiments, the scaffold domain is a V-like domain (see, e.g., Coia et al. WO 99/45110) or a domain derived from tendamistatin, which is a 74 residue, six-strand beta sheet sandwich held together by two disulfide bonds (see, e.g., McConnell and Hoess, 1995, J Mol. Biol. 250:460). For example, the loops of tendamistatin can be modified (e.g., using CDRs or hypervariable loops) or varied, e.g., to select domains that bind to a marker/antigen/cell described herein. Another exemplary scaffold domain is a beta-sandwich structure derived from the extracellular domain of CTLA-4 (see, e.g., WO 00/60070).

Other exemplary scaffold domains include but are not limited to T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains). See, e.g., US 20040009530 and U.S. Pat. No. 7,501,121, incorporated herein by reference.

In embodiments, a scaffold domain is evaluated and chosen, e.g., by one or more of the following criteria: (1) amino acid sequence, (2) sequences of several homologous domains, (3) 3-dimensional structure, and/or (4) stability data over a range of pH, temperature, salinity, organic solvent, oxidant concentration. In embodiments, the scaffold domain is a small, stable protein domain, e.g., a protein of less than 100, 70, 50, 40 or 30 amino acids. The domain may include one or more disulfide bonds or may chelate a metal, e.g., zinc.

### Antibody-Based Fusions

A variety of formats can be generated which contain additional binding entities attached to the N or C terminus of antibodies. These fusions with single chain or disulfide stabilized Fvs or Fabs result in the generation of tetravalent molecules with bivalent binding specificity for each antigen. Combinations of scFvs and scFabs with IgGs enable the production of molecules which can recognize three or more different antigens.

### Antibody-Fab Fusion

Antibody-Fab fusions are bispecific antibodies comprising a traditional antibody to a first target and a Fab to a second target fused to the C terminus of the antibody heavy chain. Commonly the antibody and the Fab will have a common light chain. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Antibody-scFv Fusion

Antibody-scFv Fusions are bispecific antibodies comprising a traditional antibody and a scFv of unique specificity fused to the C terminus of the antibody heavy chain. The scFv can be fused to the C terminus through the Heavy Chain of the scFv either directly or through a linker peptide. Antibody fusions can be produced by (1) engineering the DNA sequence of the target fusion, and (2) transfecting the target DNA into a suitable host cell to express the fusion protein. It seems like the antibody-scFv fusion may be linked by a (Gly)-Ser linker between the C-terminus of the CH3 domain and the N-terminus of the scFv, as described by Coloma, J. et al. (1997) Nature Biotech 15:159.

### Variable Domain Immunoglobulin DVD

A related format is the dual variable domain immunoglobulin (DVD), which are composed of VH and VL domains of a second specificity place upon the N termini of the V domains by shorter linker sequences.

Other exemplary multispecific antibody formats include, e.g., those described in the following US20160114057A1, US20130243775A1, US20140051833, US20130022601, US20150017187A1, US20120201746A1, US20150133638A1, US20130266568A1, US20160145340A1, WO2015127158A1, US20150203591A1, US20140322221A1, US20130303396A1, US20110293613, US20130017200A1, US20160102135A1, WO2015197598A2, WO2015197582A1, U.S. Pat. No. 9,359,437, US20150018529, WO2016115274A1, WO2016087416A1, US20080069820A1, U.S. Pat. Nos. 9,145,588B, 7,919,257, and US20150232560A1. Exemplary multispecific molecules utilizing a full antibody-Fab/scFab format include those described in the following, U.S. Pat. No. 9,382,323B2, US20140072581A1, US20140308285A1, US20130165638A1, US20130267686A1, US20140377269A1, U.S. Pat. No. 7,741,446B2, and WO1995009917A1. Exemplary multispecific molecules utilizing a domain exchange format include those described in the following, US20150315296A1, WO2016087650A1, US20160075785A1, WO2016016299A1, US20160130347A1, US20150166670, U.S. Pat. No. 8,703,132B2, US20100316645, U.S. Pat. No. 8,227,577B2, US20130078249.

### Fc-Containing Entities (Mini-Antibodies)

Fc-containing entities, also known as mini-antibodies, can be generated by fusing scFv to the C-termini of constant heavy region domain 3 (CH3-scFv) and/or to the hinge region (scFv-hinge-Fc) of an antibody with a different specificity. Trivalent entities can also be made which have disulfide stabilized variable domains (without peptide linker) fused to the C-terminus of CH3 domains of IgGs.

### Fc-Containing Multispecific Molecules

In some embodiments, the multispecific molecules disclosed herein includes an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1, IgG2, IgG3, or IgG4.

In some embodiments, the immunoglobulin chain constant region (e.g., the Fc region) is altered, e.g., mutated, to increase or decrease one or more of: Fc receptor binding, antibody glycosylation, the number of cysteine residues, effector cell function, or complement function.

In other embodiments, an interface of a first and second immunoglobulin chain constant regions (e.g., a first and a second Fc region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

In some embodiments, the multispecific molecules include a paired amino acid substitution at a position chosen from one or more of 347, 349, 350, 351, 366, 368, 370, 392, 394, 395, 397, 398, 399, 405, 407, or 409, e.g., of the Fc region of human IgG1 For example, the immunoglobulin chain constant region (e.g., Fc region) can include a paired an amino acid substitution chosen from: T366S, L368A, or Y407V (e.g., corresponding to a cavity or hole), and T366W (e.g., corresponding to a protuberance or knob).

In other embodiments, the multifunctional molecule includes a half-life extender, e.g., a human serum albumin or an antibody molecule to human serum albumin.

### Heterodimerized Antibody Molecules & Methods of Making

Various methods of producing multispecific antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined monovalent (and simultaneous) antigen binding are generated by forced heavy chain heterodimerization, combined with technologies that minimize light chain mispairing (e.g., common light chain). Forced heavy chain heterodimerization can be obtained using, e.g., knob-in-hole OR strand exchange engineered domains (SEED).

Knob-in-Hole

Knob-in-Hole as described in U.S. Pat. Nos. 5,731,116, 7,476,724 and Ridgway, J. et al. (1996) Prot. Engineering 9(7): 617-621, broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

Exemplary KiH mutations include S354C, T366W in the “knob” heavy chain and Y349C, T366S, L368A, Y407V in the “hole” heavy chain. Other exemplary KiH mutations are provided in Table 7, with additional optional stabilizing Fc cysteine mutations.

TABLE 7
Exemplary Fc KiH mutations and optional Cysteine mutations

| Position | Knob Mutation | Hole Mutation |
|---|---|---|
| T366 | T366W | T366S |
| L368 | — | L368A |
| Y407 | — | Y407V |

Additional Cysteine Mutations to form
a stabilizing disulfide bridge

| Position | Knob CH3 | Hole CH3 |
|---|---|---|
| S354 | S354C | — |
| Y349 | — | Y349C |

Other Fc mutations are provided by Igawa and Tsunoda who identified 3 negatively charged residues in the CH3 domain of one chain that pair with three positively charged residues in the CH3 domain of the other chain. These specific charged residue pairs are: E356-K439, E357-K370, D399-K409 and vice versa. By introducing at least two of the following three mutations in chain A: E356K, E357K and D399K, as well as K370E, K409D, K439E in chain B, alone or in combination with newly identified disulfide bridges, they were able to favor very efficient heterodimerization while suppressing homodimerization at the same time (Martens T et al. A novel one-armed antic-Met antibody inhibits glioblastoma growth in vivo. Clin Cancer Res 2006; 12:6144-52; PMID:17062691). Xencor defined 41 variant pairs based on combining structural calculations and sequence information that were subsequently screened for maximal heterodimerization, defining the combination of S364H, F405A (HA) on chain A and Y349T, T394F on chain B (TF) (Moore G L et al. A novel bispecific antibody format enables simultaneous bivalent and monovalent co-engagement of distinct target antigens. MAbs 2011; 3:546-57; PMID: 22123055).

Other exemplary Fc mutations to promote heterodimerization of multispecific antibodies include those described in the following references, the contents of each of which is incorporated by reference herein, WO2016071377A1, US20140079689A1, US20160194389A1, US20160257763, WO2016071376A2, WO2015107026A1, WO2015107025A1, WO2015107015A1, US20150353636A1, US20140199294A1, U.S. Pat. No. 7,750,128B2, US20160229915A1, US20150344570A1, U.S. Pat. No. 8,003,774A1, US20150337049A1, US20150175707A1, US20140242075A1, US20130195849A1, US20120149876A1, US20140200331A1, U.S. Pat. No. 9,309,311B2, U.S. Pat. No. 8,586,713, US20140037621A1, US20130178605A1, US20140363426A1, US20140051835A1 and US20110054151A1.

Stabilizing cysteine mutations have also been used in combination with KiH and other Fc heterodimerization promoting variants, see e.g., U.S. Pat. No. 7,183,076. Other exemplary cysteine modifications include, e.g., those disclosed in US20140348839A1, U.S. Pat. No. 7,855,275B2, and U.S. Pat. No. 9,000,130B2.

Strand Exchange Engineered Domains (SEED)

Heterodimeric Fc platform that support the design of bispecific and asymmetric fusion proteins by devising strand-exchange engineered domain (SEED) C(H)3 heterodimers are known. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. SEEDbodies: fusion proteins based on strand exchange engineered domain (SEED) CH3 heterodimers in an Fc analogue platform for asymmetric binders or immunofusions and bispecific antibodies. Protein Eng Des Sel 2010; 23:195-202; PMID:20299542 and U.S. Pat. No. 8,871,912. The contents of each of which are incorporated by reference herein).

Duobody

“Duobody” technology to produce bispecific antibodies with correct heavy chain pairing are known. The DuoBody technology involves three basic steps to generate stable bispecific human IgGlantibodies in a post-production exchange reaction. In a first step, two IgG1s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

Electrostatic Interactions

Methods of making multispecific antibodies using CH3 amino acid changes with charged amino acids such that homodimer formation is electrostatically unfavorable are disclosed. EP1870459 and WO 2009089004 describe other strategies for favoring heterodimer formation upon co-expression of different antibody domains in a host cell. In these methods, one or more residues that make up the heavy chain constant domain 3 (CH3), CH3-CH3 interfaces in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. No. 8,592,562B2, U.S. Pat. No. 9,200,060B2, US20140154254A1, and U.S. Pat. No. 9,358,286A1.

Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076B2, US20110177073A1, EP2847231A1, WO2016079081A1, and EP3055329A1, the contents of each of which is incorporated by reference herein.

CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (KiH) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

Amino Acid Modifications

Alternative compositions and methods of producing multispecific antibodies with correct light chain pairing include various amino acid modifications. For example, Zymeworks describes heterodimers with one or more amino acid modifications in the CH1 and/or CL domains, one or more amino acid modifications in the VH and/or VL domains, or a combination thereof, which are part of the interface between the light chain and heavy chain and create preferential pairing between each heavy chain and a desired light chain such that when the two heavy chains and two light chains of the heterodimer pair are co-expressed in a cell, the heavy chain of the first heterodimer preferentially pairs with one of the light chains rather than the other (see e.g., WO2015181805). Other exemplary methods are described in WO2016026943 (Argen-X), US20150211001, US20140072581A1, US20160039947A1, and US20150368352.

Lambda/Kappa Formats

Multispecific molecules (e.g., multispecific antibody molecules) that include the lambda light chain polypeptide and a kappa light chain polypeptides, can be used to allow for heterodimerization. Methods for generating bispecific antibody molecules comprising the lambda light chain polypeptide and a kappa light chain polypeptides are disclosed in U.S. Ser. No. 62/399,319 filed on Sep. 23, 2016, incorporated herein by reference in its entirety.

In embodiments, the multispecific molecules includes a multispecific antibody molecule, e.g., an antibody molecule comprising two binding specificities, e.g., a bispecific antibody molecule. The multispecific antibody molecule includes:

a lambda light chain polypeptide 1 (LLCP1) specific for a first epitope;

a heavy chain polypeptide 1 (HCP1) specific for the first epitope;

a kappa light chain polypeptide 2 (KLCP2) specific for a second epitope; and

a heavy chain polypeptide 2 (HCP2) specific for the second epitope.

“Lambda light chain polypeptide 1 (LLCP1)”, as that term is used herein, refers to a polypeptide comprising sufficient light chain (LC) sequence, such that when combined with a cognate heavy chain variable region, can mediate specific binding to its epitope and complex with an HCP1. In an embodiment it comprises all or a fragment of a CH1 region. In an embodiment, an LLCP1 comprises LC-CDR1, LC-CDR2, LC-CDR3, FR1, FR2, FR3, FR4, and CH1, or sufficient sequence therefrom to mediate specific binding of its epitope and complex with an HCP1. LLCP1, together with its HCP1, provide specificity for a first epitope (while KLCP2, together with its HCP2, provide specificity for a second epitope). As described elsewhere herein, LLCP1 has a higher affinity for HCP1 than for HCP2.

“Kappa light chain polypeptide 2 (KLCP2)”, as that term is used herein, refers to a polypeptide comprising sufficient light chain (LC) sequence, such that when combined with a cognate heavy chain variable region, can mediate specific binding to its epitope and complex with an HCP2. In an embodiments it comprises all or a fragment of a CH1 region. In an embodiment, a KLCP2 comprises LC-CDR1, LC-CDR2, LC-CDR3, FR1, FR2, FR3, FR4, and CH1, or sufficient sequence therefrom to mediate specific binding of its epitope and complex with an HCP2. KLCP2, together with its HCP2, provide specificity for a second epitope (while LLCP1, together with its HCP1, provide specificity for a first epitope).

“Heavy chain polypeptide 1 (HCP1)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1region. In an embodiment, it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an LLCP1, (ii) to complex preferentially, as described herein to LLCP1 as opposed to KLCP2; and (iii) to complex preferentially, as described herein, to an HCP2, as opposed to another molecule of HCP1. HCP1, together with its LLCP1, provide specificity for a first epitope (while KLCP2, together with its HCP2, provide specificity for a second epitope).

“Heavy chain polypeptide 2 (HCP2)”, as that term is used herein, refers to a polypeptide comprising sufficient heavy chain (HC) sequence, e.g., HC variable region sequence, such that when combined with a cognate LLCP1, can mediate specific binding to its epitope and complex with an HCP1. In an embodiments it comprises all or a fragment of a CH1region. In an embodiments it comprises all or a fragment of a CH2 and/or CH3 region. In an embodiment an HCP1 comprises HC-CDR1, HC-CDR2, HC-CDR3, FR1, FR2, FR3, FR4, CH1, CH2, and CH3, or sufficient sequence therefrom to: (i) mediate specific binding of its epitope and complex with an KLCP2, (ii) to complex preferentially, as described herein to KLCP2 as opposed to LLCP1; and (iii) to complex preferentially, as described herein, to an HCP1, as opposed to another molecule of HCP2. HCP2, together with its KLCP2, provide specificity for a second epitope (while LLCP1, together with its HCP1, provide specificity for a first epitope).

In some embodiments of the multispecific antibody molecule disclosed herein:

LLCP1 has a higher affinity for HCP1 than for HCP2; and/or

KLCP2 has a higher affinity for HCP2 than for HCP1.

In embodiments, the affinity of LLCP1 for HCP1 is sufficiently greater than its affinity for HCP2, such that under preselected conditions, e.g., in aqueous buffer, e.g., at pH 7, in saline, e.g., at pH 7, or under physiological conditions, at least 75%, 80, 90, 95, 98, 99, 99.5, or 99.9% of the multispecific antibody molecule molecules have a LLCP1complexed, or interfaced with, a HCP.

In some embodiments of the multispecific antibody molecule disclosed herein:

the HCP1 has a greater affinity for HCP2, than for a second molecule of HCP1; and/or

the HCP2 has a greater affinity for HCP1, than for a second molecule of HCP2.

In embodiments, the affinity of HCP1 for HCP2 is sufficiently greater than its affinity for a second molecule of HCP1, such that under preselected conditions, e.g., in aqueous buffer, e.g., at pH 7, in saline, e.g., at pH 7, or under physiological conditions, at least 75%, 80, 90, 95, 98, 99 99.5 or 99.9% of the multispecific antibody molecule molecules have a HCP1complexed, or interfaced with, a HCP2.

In another aspect, disclosed herein is a method for making, or producing, a multispecific antibody molecule. The method includes:

(i) providing a first heavy chain polypeptide (e.g., a heavy chain polypeptide comprising one, two, three or all of a first heavy chain variable region (first VH), a first CH1, a first heavy chain constant region (e.g., a first CH2, a first CH3, or both));

(ii) providing a second heavy chain polypeptide (e.g., a heavy chain polypeptide comprising one, two, three or all of a second heavy chain variable region (second VH), a second CH1, a second heavy chain constant region (e.g., a second CH2, a second CH3, or both));

(iii) providing a lambda chain polypeptide (e.g., a lambda light variable region (VLX), a lambda light constant chain (VLX), or both) that preferentially associates with the first heavy chain polypeptide (e.g., the first VH); and

(iv) providing a kappa chain polypeptide (e.g., a lambda light variable region (VLK), a lambda light constant chain (VLK), or both) that preferentially associates with the second heavy chain polypeptide (e.g., the second VH),

under conditions where (i)-(iv) associate.

In embodiments, the first and second heavy chain polypeptides form an Fc interface that enhances heterodimerization.

In embodiments, (i)-(iv) (e.g., nucleic acid encoding (i)-(iv)) are introduced in a single cell, e.g., a single mammalian cell, e.g., a CHO cell. In embodiments, (i)-(iv) are expressed in the cell.

In embodiments, (i)-(iv) (e.g., nucleic acid encoding (i)-(iv)) are introduced in different cells, e.g., different mammalian cells, e.g., two or more CHO cell. In embodiments, (i)-(iv) are expressed in the cells.

In one embodiments, the method further comprises purifying a cell-expressed antibody molecule, e.g., using a lambda- and/or-kappa-specific purification, e.g., affinity chromatography.

In embodiments, the method further comprises evaluating the cell-expressed multispecific antibody molecule. For example, the purified cell-expressed multispecific antibody molecule can be analyzed by techniques known in the art, include mass spectrometry. In one embodiment, the purified cell-expressed antibody molecule is cleaved, e.g., digested with papain to yield the Fab moieties and evaluated using mass spectrometry.

In embodiments, the method produces correctly paired kappa/lambda multispecific, e.g., bispecific, antibody molecules in a high yield, e.g., at least 75%, 80, 90, 95, 98, 99 99.5 or 99.9%.

In other embodiments, the multispecific, e.g., a bispecific, antibody molecule that includes:

(i) a first heavy chain polypeptide (HCP1) (e.g., a heavy chain polypeptide comprising one, two, three or all of a first heavy chain variable region (first VH), a first CH1, a first heavy chain constant region (e.g., a first CH2, a first CH3, or both)), e.g., wherein the HCP1 binds to a first epitope;

(ii) a second heavy chain polypeptide (HCP2) (e.g., a heavy chain polypeptide comprising one, two, three or all of a second heavy chain variable region (second VH), a second CH1, a second heavy chain constant region (e.g., a second CH2, a second CH3, or both)), e.g., wherein the HCP2 binds to a second epitope;

(iii) a lambda light chain polypeptide (LLCP1) (e.g., a lambda light variable region (VL1), a lambda light constant chain (VL1), or both) that preferentially associates with the first heavy chain polypeptide (e.g., the first VH), e.g., wherein the LLCP1 binds to a first epitope; and

(iv) a kappa light chain polypeptide (KLCP2) (e.g., a lambda light variable region (VLk), a lambda light constant chain (VLk), or both) that preferentially associates with the second heavy chain polypeptide (e.g., the second VH), e.g., wherein the KLCP2 binds to a second epitope.

In embodiments, the first and second heavy chain polypeptides form an Fc interface that enhances heterodimerization. In embodiments, the multispecific antibody molecule has a first binding specificity that includes a hybrid VL1-CL1 heterodimerized to a first heavy chain variable region connected to the Fc constant, CH2-CH3 domain (having a knob modification) and a second binding specificity that includes a hybrid VLk-CLk heterodimerized to a second heavy chain variable region connected to the Fc constant, CH2-CH3 domain (having a hole modification).

### Nucleic Acids

The invention also features nucleic acids comprising nucleotide sequences that encode heavy and light chain variable regions and CDRs or hypervariable loops of the antibody molecules, as described herein. For example, the invention features a first and second nucleic acid encoding heavy and light chain variable regions, respectively, of an antibody molecule chosen from one or more of the antibody molecules disclosed herein. The nucleic acid can comprise a nucleotide sequence as set forth in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein.

In certain embodiments, the nucleic acid can comprise a nucleotide sequence encoding at least one, two, or three CDRs or hypervariable loops from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In other embodiments, the nucleic acid can comprise a nucleotide sequence encoding at least one, two, or three CDRs or hypervariable loops from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In yet another embodiment, the nucleic acid can comprise a nucleotide sequence encoding at least one, two, three, four, five, or six CDRs or hypervariable loops from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs or hypervariable loops from a heavy chain variable region having the nucleotide sequence as set forth in the tables herein, a sequence substantially homologous thereto (e.g., a sequence at least about 85%, 90%, 95%, 99% or more identical thereto, and/or capable of hybridizing under the stringency conditions described herein). In another embodiment, the nucleic acid can comprise a nucleotide sequence encoding at least one, two, or three CDRs or hypervariable loops from a light chain variable region having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein). In yet another embodiment, the nucleic acid can comprise a nucleotide sequence encoding at least one, two, three, four, five, or six CDRs or hypervariable loops from heavy and light chain variable regions having the nucleotide sequence as set forth in the tables herein, or a sequence substantially homologous thereto (e.g., a sequence at least about 85%, 90%, 95%, 99% or more identical thereto, and/or capable of hybridizing under the stringency conditions described herein).

In another aspect, the application features host cells and vectors containing the nucleic acids described herein. The nucleic acids may be present in a single vector or separate vectors present in the same host cell or separate host cell, as described in more detail hereinbelow.

### Vectors

Further provided herein are vectors comprising the nucleotide sequences encoding an antibody molecule described herein. In one embodiment, the vectors comprise nucleotides encoding an antibody molecule described herein. In one embodiment, the vectors comprise the nucleotide sequences described herein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

In another aspect, the application features host cells and vectors containing the nucleic acids described herein. The nucleic acids may be present in a single vector or separate vectors present in the same host cell or separate host cell. The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. The invention also provides host cells comprising a nucleic acid encoding an antibody molecule as described herein.

In one embodiment, the host cells are genetically engineered to comprise nucleic acids encoding the antibody molecule.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The invention also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### Uses and Combination Therapies

Methods described herein include treating a cancer in a subject by using a multispecific molecule described herein, e.g., using a pharmaceutical composition described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, myelofibrosis, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the multispecific molecules (or pharmaceutical composition) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or multispecific molecules) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the multispecific molecules or pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation. In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The multispecific molecules disclosed herein can be used in combination with a second therapeutic agent or procedure.

In embodiments, the multispecific molecule and the second therapeutic agent or procedure are administered/performed after a subject has been diagnosed with a cancer, e.g., before the cancer has been eliminated from the subject. In embodiments, the multispecific molecule and the second therapeutic agent or procedure are administered/performed simultaneously or concurrently. For example, the delivery of one treatment is still occurring when the delivery of the second commences, e.g., there is an overlap in administration of the treatments. In other embodiments, the multispecific molecule and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the multispecific molecule is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the multispecific molecule and the therapy, e.g., the cancer therapy, can be sequential (with or without overlap) or simultaneous. Administration of the multispecific molecule can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J. Clin. 61:250-281).

### Anti-Cancer Therapies

In other embodiments, the multispecific molecule is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, the multispecific molecule is administered in conjunction with a biologic. Biologics useful in the treatment of cancers are known in the art and a binding molecule of the invention may be administered, for example, in conjunction with such known biologics. For example, the FDA has approved the following biologics for the treatment of breast cancer: HERCEPTIN® (trastuzumab, Genentech Inc., South San Francisco, Calif.; a humanized monoclonal antibody that has anti-tumor activity in HER2-positive breast cancer); FASLODEX® (fulvestrant, AstraZeneca Pharmaceuticals, LP, Wilmington, Del.; an estrogen-receptor antagonist used to treat breast cancer); ARIMIDEX® (anastrozole, AstraZeneca Pharmaceuticals, LP; a nonsteroidal aromatase inhibitor which blocks aromatase, an enzyme needed to make estrogen); Aromasin® (exemestane, Pfizer Inc., New York, N.Y.; an irreversible, steroidal aromatase inactivator used in the treatment of breast cancer); FEMARA® (letrozole, Novartis Pharmaceuticals, East Hanover, N.J.; a nonsteroidal aromatase inhibitor approved by the FDA to treat breast cancer); and NOLVADEX® (tamoxifen, AstraZeneca Pharmaceuticals, LP; a nonsteroidal antiestrogen approved by the FDA to treat breast cancer). Other biologics with which the binding molecules of the invention may be combined include: AVASTIN® (bevacizumab, Genentech Inc.; the first FDA-approved therapy designed to inhibit angiogenesis); and ZEVALIN® (ibritumomab tiuxetan, Biogen Idec, Cambridge, Mass.; a radiolabeled monoclonal antibody currently approved for the treatment of B-cell lymphomas).

In addition, the FDA has approved the following biologics for the treatment of colorectal cancer: AVASTIN®; ERBITUX® (cetuximab, ImClone Systems Inc., New York, N.Y., and Bristol-Myers Squibb, New York, N.Y.; is a monoclonal antibody directed against the epidermal growth factor receptor (EGFR)); GLEEVEC® (imatinib mesylate; a protein kinase inhibitor); and ERGAMISOL® (levamisole hydrochloride, Janssen Pharmaceutica Products, LP, Titusville, N.J.; an immunomodulator approved by the FDA in 1990 as an adjuvant treatment in combination with 5-fluorouracil after surgical resection in patients with Dukes' Stage C colon cancer).

For the treatment of lung cancer, exemplary biologics include TARCEVA® (erlotinib HCL, OSI Pharmaceuticals Inc., Melville, N.Y.; a small molecule designed to target the human epidermal growth factor receptor 1 (HER1) pathway).

For the treatment of multiple myeloma, exemplary biologics include VELCADE® Velcade (bortezomib, Millennium Pharmaceuticals, Cambridge Mass.; a proteasome inhibitor). Additional biologics include THALIDOMID® (thalidomide, Clegene Corporation, Warren, N.J.; an immunomodulatory agent and appears to have multiple actions, including the ability to inhibit the growth and survival of myeloma cells and anti-angiogenesis).

Additional exemplary cancer therapeutic antibodies include, but are not limited to, 3F8, abagovomab, adecatumumab, afutuzumab, alacizumab pegol, alemtuzumab (CAMPATH®, MABCAMPATH®), altumomab pentetate (HYBRI-CEAKER®), anatumomab mafenatox, anrukinzumab (IMA-638), apolizumab, arcitumomab (CEA-SCAN®), bavituximab, bectumomab (LYMPHOSCAN®), belimumab (BENLYSTA®, LYMPHOSTAT-B®), besilesomab (SCINTIMUN®), bevacizumab (AVASTIN®), bivatuzumab mertansine, blinatumomab, brentuximab vedotin, cantuzumab mertansine, capromab pendetide (PROSTASCINT®), catumaxomab (REMOVAB®), CC49, cetuximab (C225, ERBITUX®), citatuzumab bogatox, cixutumumab, clivatuzumab tetraxetan, conatumumab, dacetuzumab, denosumab (PROLIA®), detumomab, ecromeximab, edrecolomab (PANOREX®), elotuzumab, epitumomab cituxetan, epratuzumab, ertumaxomab (REXOMUN®), etaracizumab, farletuzumab, figitumumab, fresolimumab, galiximab, gemtuzumab ozogamicin (MYLOTARG®), girentuximab, glembatumumab vedotin, ibritumomab (ibritumomab tiuxetan, ZEVALIN®), igovomab (INDIMACIS-125®), intetumumab, inotuzumab ozogamicin, ipilimumab, iratumumab, labetuzumab (CEA-CIDE®), lexatumumab, lintuzumab, lucatumumab, lumiliximab, mapatumumab, matuzumab, milatuzumab, minretumomab, mitumomab, nacolomab tafenatox, naptumomab estafenatox, necitumumab, nimotuzumab (THERACIM®, THERALOC®), nofetumomab merpentan (VERLUMA®), ofatumumab (ARZERRA®), olaratumab, oportuzumab monatox, oregovomab (OVAREX®), panitumumab (VECTIBIX®), pemtumomab (THERAGYN®), pertuzumab (OMNITARG®), pintumomab, pritumumab, ramucirumab, ranibizumab (LUCENTIS®), rilotumumab, rituximab (MABTHERA®, RITUXAN®), robatumumab, satumomab pendetide, sibrotuzumab, siltuximab, sontuzumab, tacatuzumab tetraxetan (AFP-CIDE®), taplitumomab paptox, tenatumomab, TGN1412, ticilimumab (tremelimumab), tigatuzumab, TNX-650, tositumomab (BEXXAR®), trastuzumab (HERCEPTIN®), tremelimumab, tucotuzumab celmoleukin, veltuzumab, volociximab, votumumab (HUMASPECT®), zalutumumab (HUMAX-EGFR®), and zanolimumab (HUMAX-CD4®).

In other embodiments, the multispecific molecule is administered in combination with a viral cancer therapeutic agent. Exemplary viral cancer therapeutic agents include, but not limited to, vaccinia virus (vvDD-CDSR), carcinoembryonic antigen-expressing measles virus, recombinant vaccinia virus (TK-deletion plus GM-CSF), Seneca Valley virus-001, Newcastle virus, coxsackie virus A21, GL-ONC1, EBNA1 C-terminal/LMP2 chimeric protein-expressing recombinant modified vaccinia Ankara vaccine, carcinoembryonic antigen-expressing measles virus, G207 oncolytic virus, modified vaccinia virus Ankara vaccine expressing p53, OncoVEX GM-CSF modified herpes-simplex 1 virus, fowlpox virus vaccine vector, recombinant vaccinia prostate-specific antigen vaccine, human papillomavirus 16/18 L1 virus-like particle/AS04 vaccine, MVA-EBNA1/LMP2 Inj. vaccine, quadrivalent HPV vaccine, quadrivalent human papillomavirus (types 6, 11, 16, 18) recombinant vaccine (GARDASIL®), recombinant fowlpox-CEA(6D)/TRICOM vaccine; recombinant vaccinia-CEA(6D)-TRICOM vaccine, recombinant modified vaccinia Ankara-5T4 vaccine, recombinant fowlpox-TRICOM vaccine, oncolytic herpes virus NV1020, HPV L1 VLP vaccine V504, human papillomavirus bivalent (types 16 and 18) vaccine (CERVARIX®), herpes simplex virus HF10, Ad5CMV-p53 gene, recombinant vaccinia DF3/MUC1 vaccine, recombinant vaccinia-MUC-1 vaccine, recombinant vaccinia-TRICOM vaccine, ALVAC MART-1 vaccine, replication-defective herpes simplex virus type I (HSV-1) vector expressing human Preproenkephalin (NP2), wild-type reovirus, reovirus type 3 Dearing (REOLYSIN®), oncolytic virus HSV1716, recombinant modified vaccinia Ankara (MVA)-based vaccine encoding Epstein-Barr virus target antigens, recombinant fowlpox-prostate specific antigen vaccine, recombinant vaccinia prostate-specific antigen vaccine, recombinant vaccinia-B7.1 vaccine, rAd-p53 gene, Ad5-delta24RGD, HPV vaccine 580299, JX-594 (thymidine kinase-deleted vaccinia virus plus GM-CSF), HPV-16/18 L1/AS04, fowlpox virus vaccine vector, vaccinia-tyrosinase vaccine, MEDI-517 HPV-16/18 VLP AS04 vaccine, adenoviral vector containing the thymidine kinase of herpes simplex virus TK99UN, HspE7, FP253/Fludarabine, ALVAC(2) melanoma multi-antigen therapeutic vaccine, ALVAC-hB7.1, canarypox-hIL-12 melanoma vaccine, Ad-REIC/Dkk-3, rAd-IFN SCH 721015, TIL-Ad-INFg, Ad-ISF35, and coxsackievirus A21 (CVA21, CAVATAK®).

In other embodiments, the multispecific molecule is administered in combination with a nanopharmaceutical. Exemplary cancer nanopharmaceuticals include, but not limited to, ABRAXANE® (paclitaxel bound albumin nanoparticles), CRLX101 (CPT conjugated to a linear cyclodextrin-based polymer), CRLX288 (conjugating docetaxel to the biodegradable polymer poly (lactic-co-glycolic acid)), cytarabine liposomal (liposomal Ara-C, DEPOCYT™), daunorubicin liposomal (DAUNOXOME®), doxorubicin liposomal (DOXIL®, CAELYX®), encapsulated-daunorubicin citrate liposome (DAUNOXOME®), and PEG anti-VEGF aptamer (MACUGEN®).

In some embodiments, the multispecific molecule is administered in combination with paclitaxel or a paclitaxel formulation, e.g., TAXOL®, protein-bound paclitaxel (e.g., ABRAXANE®). Exemplary paclitaxel formulations include, but are not limited to, nanoparticle albumin-bound paclitaxel (ABRAXANE®, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., Bioorganic & Medicinal Chemistry Letters (2007) 17:617-620).

Exemplary RNAi and antisense RNA agents for treating cancer include, but not limited to, CALAA-01, siG12D LODER (Local Drug EluteR), and ALN-VSP02.

Other cancer therapeutic agents include, but not limited to, cytokines (e.g., aldesleukin (IL-2, Interleukin-2, PROLEUKIN®), alpha Interferon (IFN-alpha, Interferon alfa, INTRON® A (Interferon alfa-2b), ROFERON-A® (Interferon alfa-2a)), Epoetin alfa (PROCRIT®), filgrastim (G-CSF, Granulocyte—Colony Stimulating Factor, NEUPOGEN®), GM-CSF (Granulocyte Macrophage Colony Stimulating Factor, sargramostim, LEUKINE™), IL-11 (Interleukin-11, oprelvekin, NEUMEGA®), Interferon alfa-2b (PEG conjugate) (PEG interferon, PEG-INTRON™), and pegfilgrastim (NEULASTA™)), hormone therapy agents (e.g., aminoglutethimide (CYTADREN®), anastrozole (ARIMIDEX®), bicalutamide (CASODEX®), exemestane (AROMASIN®), fluoxymesterone (HALOTESTIN®), flutamide (EULEXIN®), fulvestrant (FASLODEX®), goserelin (ZOLADEX®), letrozole (FEMARA®), leuprolide (ELIGARD™, LUPRON®, LUPRON DEPOT®, VIADUR™), megestrol (megestrol acetate, MEGACE®), nilutamide (ANANDRON®, NILANDRON®), octreotide (octreotide acetate, SANDOSTATIN®, SANDOSTATIN LAR®), raloxifene (EVISTA®), romiplostim (NPLATE®), tamoxifen (NOVALDEX®), and toremifene (FARESTON®)), phospholipase A2 inhibitors (e.g., anagrelide (AGRYLIN®)), biologic response modifiers (e.g., BCG (THERACYS®, TICE®), and Darbepoetin alfa (ARANESP®)), target therapy agents (e.g., bortezomib (VELCADE®), dasatinib (SPRYCEL™), denileukin diftitox (ONTAK®), erlotinib (TARCEVA®), everolimus (AFINITOR®), gefitinib (IRESSA®), imatinib mesylate (STI-571, GLEEVEC™), lapatinib (TYKERB®), sorafenib (NEXAVAR®), and SU11248 (sunitinib, SUTENT®)), immunomodulatory and antiangiogenic agents (e.g., CC-5013 (lenalidomide, REVLIMID®), and thalidomide (THALOMID®)), glucocorticosteroids (e.g., cortisone (hydrocortisone, hydrocortisone sodium phosphate, hydrocortisone sodium succinate, ALA-CORT®, HYDROCORT ACETATE®, hydrocortone phosphate LANACORT®, SOLU-CORTEF®), decadron (dexamethasone, dexamethasone acetate, dexamethasone sodium phosphate, DEXASONE®, DIODEX®, HEXADROL®, MAXIDEX®), methylprednisolone (6-methylprednisolone, methylprednisolone acetate, methylprednisolone sodium succinate, DURALONE®, MEDRALONE®, MEDROL®, M-PREDNISOL®, SOLU-MEDROL®), prednisolone (DELTA-CORTEF®, ORAPRED®, PEDIAPRED®, PRELONE®), and prednisone (DELTASONE®, LIQUID PRED®, METICORTEN®, ORASONE®)), and bisphosphonates (e.g., pamidronate (AREDIA®), and zoledronic acid (ZOMETA®))

In some embodiments, the multispecific molecule is used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., an antibody against VEGF, a VEGF trap, a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the AHCM agent is selected from the group consisting of: axitinib (AG013736), bosutinib (SKI-606), cediranib (RECENTIN™, AZD2171), dasatinib (SPRYCEL®, BMS-354825), erlotinib (TARCEVA®), gefitinib (IRESSA®)), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, Pazopanib Hydrochloride, PD173074,nSorafenib Tosylate(Bay 43-9006), SU 5402, TSU-68(SU6668), vatalanib, XL880 (GSK1363089, EXEL-2880). Selected tyrosine kinase inhibitors are chosen from sunitinib, erlotinib, gefitinib, or sorafenib. In one embodiment, the tyrosine kinase inhibitor is sunitinib.

In one embodiment, the multispecific molecule is administered in combination with one of more of: an anti-angiogenic agent, or a vascular targeting agent or a vascular disrupting agent. Exemplary anti-angiogenic agents include, but are not limited to, VEGF inhibitors (e.g., anti-VEGF antibodies (e.g., bevacizumab); VEGF receptor inhibitors (e.g., itraconazole); inhibitors of cell proliferatin and/or migration of endothelial cells (e.g., carboxyamidotriazole, TNP-470); inhibitors of angiogenesis stimulators (e.g., suramin), among others. A vascular-targeting agent (VTA) or vascular disrupting agent (VDA) is designed to damage the vasculature (blood vessels) of cancer tumors causing central necrosis (reviewed in, e.g., Thorpe, P. E. (2004) Clin. Cancer Res. Vol. 10:415-427). VTAs can be small-molecule. Exemplary small-molecule VTAs include, but are not limited to, microtubule destabilizing drugs (e.g., combretastatin A-4 disodium phosphate (CA4P), ZD6126, AVE8062, Oxi 4503); and vadimezan (ASA404).

### Immune Checkpoint Inhibitors

In other embodiments, methods described herein comprise use of an immune checkpoint inhibitor in combination with the multispecific molecule. The methods can be used in a therapeutic protocol in vivo.

In embodiments, an immune checkpoint inhibitor inhibits a checkpoint molecule. Exemplary checkpoint molecules include but are not limited to CTLA4, PD1, PD-L1, PD-L2, TIM3, LAG3, CD160, 2B4, CD80, CD86, B7-H3 (CD276), B7-H4 (VTCN1), HVEM (TNFRSF14 or CD270), BTLA, KIR, MHC class I, MHC class II, GALS, VISTA, BTLA, TIGIT, LAIR1, and A2aR. See, e.g., Pardoll. Nat. Rev. Cancer 12.4(2012):252-64, incorporated herein by reference.

In embodiments, the immune checkpoint inhibitor is a PD-1 inhibitor, e.g., an anti-PD-1 antibody such as Nivolumab, Pembrolizumab or Pidilizumab. Nivolumab (also called MDX-1106, MDX-1106-04, ONO-4538, or BMS-936558) is a fully human IgG4 monoclonal antibody that specifically inhibits PD1. See, e.g., U.S. Pat. No. 8,008,449 and WO2006/121168. Pembrolizumab (also called Lambrolizumab, MK-3475, MK03475, SCH-900475 or KEYTRUDA®; Merck) is a humanized IgG4 monoclonal antibody that binds to PD-1. See, e.g., Hamid, O. et al. (2013) New England Journal of Medicine 369 (2): 134-44, U.S. Pat. No. 8,354,509 and WO2009/114335. Pidilizumab (also called CT-011 or Cure Tech) is a humanized IgG1k monoclonal antibody that binds to PD1. See, e.g., WO2009/101611. In one embodiment, the inhibitor of PD-1 is an antibody molecule having a sequence substantially identical or similar thereto, e.g., a sequence at least 85%, 90%, 95% identical or higher to the sequence of Nivolumab, Pembrolizumab or Pidilizumab. Additional anti-PD1 antibodies, e.g., AMP 514 (Amplimmune), are described, e.g., in U.S. Pat. No. 8,609,089, US 2010028330, and/or US 20120114649.

In some embodiments, the PD-1 inhibitor is an immunoadhesin, e.g., an immunoadhesin comprising an extracellular/PD-1 binding portion of a PD-1 ligand (e.g., PD-L1 or PD-L2) that is fused to a constant region (e.g., an Fc region of an immunoglobulin). In embodiments, the PD-1 inhibitor is AMP-224 (B7-DCIg, e.g., described in WO2011/066342 and WO2010/027827), a PD-L2 Fc fusion soluble receptor that blocks the interaction between B7-H1 and PD-1.

In embodiments, the immune checkpoint inhibitor is a PD-L1 inhibitor, e.g., an antibody molecule. In some embodiments, the PD-L1 inhibitor is YW243.55.570, MPDL3280A, MEDI-4736, MSB-0010718C, or MDX-1105. In some embodiments, the anti-PD-L1 antibody is MSB0010718C (also called A09-246-2; Merck Serono), which is a monoclonal antibody that binds to PD-L1. Exemplary humanized anti-PD-L1 antibodies are described, e.g., in WO2013/079174. In one embodiment, the PD-L1 inhibitor is an anti-PD-L1 antibody, e.g., YW243.55.S70. The YW243.55.S70 antibody is described, e.g., in WO 2010/077634. In one embodiment, the PD-L1 inhibitor is MDX-1105 (also called BMS-936559), which is described, e.g., in WO2007/005874. In one embodiment, the PD-L1 inhibitor is MDPL3280A (Genentech/Roche), which is a human Fc-optimized IgG1 monoclonal antibody against PD-L1. See, e.g., U.S. Pat. No. 7,943,743 and U.S. Publication No.: 20120039906. In one embodiment, the inhibitor of PD-L1 is an antibody molecule having a sequence substantially identical or similar thereto, e.g., a sequence at least 85%, 90%, 95% identical or higher to the sequence of YW243.55.570, MPDL3280A, MEDI-4736, MSB-0010718C, or MDX-1105.

In embodiments, the immune checkpoint inhibitor is a PD-L2 inhibitor, e.g., AMP-224 (which is a PD-L2 Fc fusion soluble receptor that blocks the interaction between PD1 and B7-H1. See, e.g., WO2010/027827 and WO2011/066342.

In one embodiment, the immune checkpoint inhibitor is a LAG-3 inhibitor, e.g., an anti LAG-3 antibody molecule. In embodiments, the anti-LAG-3 antibody is BMS-986016 (also called BMS986016; Bristol-Myers Squibb). BMS-986016 and other humanized anti-LAG-3 antibodies are described, e.g., in US 2011/0150892, WO2010/019570, and WO2014/008218. In embodiments, the immune checkpoint inhibitor is a TIM-3 inhibitor, e.g., anti-TIM3 antibody molecule, e.g., described in U.S. Pat. No. 8,552,156, WO 2011/155607, EP 2581113 and U.S. Publication No.: 2014/044728.

In embodiments, the immune checkpoint inhibitor is a CTLA-4 inhibitor, e.g., anti-CTLA-4 antibody molecule. Exemplary anti-CTLA4 antibodies include Tremelimumab (IgG2 monoclonal antibody from Pfizer, formerly known as ticilimumab, CP-675,206); and Ipilimumab (also called MDX-010, CAS No. 477202-00-9). Other exemplary anti-CTLA-4 antibodies are described, e.g., in U.S. Pat. No. 5,811,097.

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A multispecific molecule comprising a first targeting moiety that binds to MPL and a second targeting moiety that binds to a phosphatase, e.g., a protein tyrosine phosphatase (PTP), e.g., a receptor protein tyrosine phosphatase (RPTP).

2. The multispecific molecule of claim 1, wherein the phosphatase and MPL are expressed in a same cell, e.g., a myelofibrosis cell.

3. The multispecific molecule of claim 1 or 2, wherein the phosphatase can dephosphorylate MPL or a molecule that interacts directly or indirectly with MPL (e.g., a tyrosine kinase that interacts directly or indirectly with MPL, e.g., JAK2 or Src).

4. The multispecific molecule of any one of claims 1-3, wherein the phosphatase is selected from the group consisting of CD45, RPTPμ, RPTPκ, RPTPρ, RPTPλ, leukocyte antigen-related tyrosine phosphatase (LAR), RPTPσ, RPTPδ, RPTPβ, CD148, SAP1, RPTPO, RPTPQ/PTPS31, RPTPα, RPTPε, RPTPζ, RPTPγ, PC-PTP, IA2, and IA2β.

5. The multispecific molecule of any one of claims 1-3, wherein the phosphatase is CD45, optionally wherein the targeting moiety that binds to a phosphatase binds to one or more of CD45RA, CD45RB, CD45RC, CD45RAB, CD45RAC, CD45RBC, CD45RO, or CD45R (ABC).

6. The multispecific molecule of any one of claims 1-5, wherein the first targeting moiety that binds to MPL comprises:
(i) one, two, or three CDRs from any of the heavy chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 1;
(ii) a heavy chain variable domain sequence chosen from any of the heavy chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);
(iii) one, two, or three CDRs from any of the light chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 1; or
(iv) a light chain variable domain sequence chosen from any of the light chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

7. The multispecific molecule of any one of claim 1-3, 5, or 6, wherein the second targeting moiety that binds to a phosphatase binds to CD45, wherein the second targeting moiety comprises:
(i) one, two, or three CDRs from any of the heavy chain variable domain sequences of Table 3, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 3;
(ii) a heavy chain variable domain sequence chosen from any of the heavy chain variable domain amino acid sequences of Table 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);
(iii) one, two, or three CDRs from any of the light chain variable domain sequences of Table 3, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 3; or
(iv) a light chain variable domain sequence chosen from any of the light chain variable domain amino acid sequences of Table 3, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

8. The multispecific molecule of any one of claim 1-3 or 6, wherein the second targeting moiety that binds to a phosphatase binds to CD148, wherein the second targeting moiety comprises:
(i) one, two, or three CDRs from any of the heavy chain variable domain sequences of Table 4, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 4;
(ii) a heavy chain variable domain sequence chosen from any of the heavy chain variable domain amino acid sequences of Table 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);
(iii) one, two, or three CDRs from any of the light chain variable domain sequences of Table 4, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 4; or
(iv) a light chain variable domain sequence chosen from any of the light chain variable domain amino acid sequences of Table 4, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

9. The multispecific molecule of any one of claim 1-3 or 6, wherein the second targeting moiety that binds to a phosphatase binds to LAR, wherein the second targeting moiety comprises:
(i) one, two, or three CDRs from any of the heavy chain variable domain sequences of Table 5, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 5;
(ii) a heavy chain variable domain sequence chosen from any of the heavy chain variable domain amino acid sequences of Table 5, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions);
(iii) one, two, or three CDRs from any of the light chain variable domain sequences of Table 5, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 5; or
(iv) a light chain variable domain sequence chosen from any of the light chain variable domain amino acid sequences of Table 5, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

10. The multispecific molecule of any one of claims 1-9, wherein the multispecific molecule further comprises an effector moiety, e.g., wherein the effector moiety is chosen from one or more of an immune cell engager, a cytokine molecule, a cytokine antagonist, e.g., a TGF-β antagonist, an enzyme, a toxin, or a labeling agent.

11. The multispecific molecule of claim 10, wherein the effector moiety is an immune cell engager (e.g., an anti-CD3 antibody molecule).

12. The multispecific molecule of claim 10, wherein the effector moiety is a TGF-β antagonist, e.g., a polypeptide comprising a TGFβ receptor, or functional fragment or variant thereof, that is capable of binding TGFβ, e.g., an extracellular domain of TGFβ receptor type I or an extracellular domain of TGFβ receptor type II.

13. The multispecific molecule of claim 12, wherein the TGFβ antagonist comprises any amino acid sequence of Table 6, or an amino acid sequence substantially identical thereto (e.g., 75%, 8-0%, 85%, 90%, 95%, or 99.9% identical thereto), or having at least one amino acid alteration, but not more than five, ten, fifteen, or twenty alterations (e.g., substitutions, deletions, or insertions, e.g., conservative substitutions).

14. A multispecific molecule (e.g., a multispecific or multifunctional antibody molecule), comprising a first MPL-targeting moiety, wherein the first MPL-targeting moiety binds to MPL.

15. The multispecific molecule of claim 14, wherein the multispecific molecule reduces, e.g., inhibits, an MPL activity.

16. The multispecific molecule of claim 14 or 15, comprising a second MPL-targeting moiety that binds to MPL.

17. The multispecific molecule of claim 16, wherein the first and the second MPL-targeting moieties bind the same epitope (e.g., bind overlapping epitopes).

18. The multispecific molecule of claim 16, wherein the first and the second MPL-targeting moieties bind different epitopes on a single MPL protein (e.g., bind non overlapping epitopes).

19. The multispecific molecule of claim 18, which is a biparatopic antibody molecule.

20. An MPL-binding molecule comprising a single MPL-targeting moiety, e.g., a half-arm antibody against MPL (e.g., a Fab or single chain Fv fused to a first immunoglobulin constant domain (e.g., a first Fc constant region (e.g., a first CH2-CH3)), wherein the MPL-binding molecule reduces, e.g., inhibits, an MPL activity.

21. The MPL-binding molecule of claim 20, which is monovalent.

22. The MPL-binding molecule of claim 20 or 21, further comprising a second immunoglobulin constant domain, e.g., a second heavy chain constant region, e.g., a second Fc constant region, e.g., a second CH2-CH3.

23. The multispecific molecule or MPL-binding molecule of any one of claims 14-22, which further comprises one or more of an immune cell engager, a cytokine molecule, or a tumor targeting molecule (e.g., a tumor targeting molecule that targets a tumor target other than MPL).

24. The multispecific molecule or MPL-binding molecule of claim 23, wherein the tumor targeting molecule is an anti-CD41 antibody molecule or an anti-CD177 antibody molecule.

25. The multispecific molecule or MPL-binding molecule of any one of claims 14-24, further comprising an anti-PDL1 antibody molecule, an anti-CD3 antibody molecule, an anti-TGFβ antibody molecule, a TGFβ trap polypeptide (e.g., a polypeptide comprising a portion of TGFβ receptor that is capable of binding TGFβ), an anti-IL1β antibody molecule, an IL1β trap polypeptide (e.g., a polypeptide comprising a portion of IL113 receptor that is capable of binding IL1β), an anti-CXCL10 antibody molecule, an anti-MS4A3 antibody molecule, an anti-OLFM4 antibody molecule, an anti-CD66b antibody molecule, an anti-cKit antibody molecule, an anti-FLT3 antibody molecule, or an anti-CD133 antibody molecule (or any combination thereof).

26. The multispecific molecule or MPL-binding molecule of any one of claims 14-25, which binds to the extracellular domain of MPL.

27. The multispecific molecule or MPL-binding molecule of any one of claims 14-26, which prevents the association of the MPL bound to the molecule with a second MPL protein.

28. The multispecific molecule or MPL-binding molecule of any one of claims 14-27, which reduces (e.g., prevents) one, two or more of MPL protein dimerization, intracellular phosphorylation or activation of the JAK2 kinase pathway.

29. The multispecific molecule or MPL-binding molecule of any one of claims 15-28, wherein the MPL activity is reduced in the presence of an MPL ligand, e.g., TPO.

30. The multispecific molecule or MPL-binding molecule of any one of claim 16-19 or 23-29, wherein the affinity, e.g., the combined affinity, for the MPL of the first and the second MPL-targeting moieties is equal to or greater than the affinity of each targeting moiety (either alone or as part of the multispecific molecule) for its corresponding antigen binding site.

31. The multispecific molecule or MPL-binding molecule of claim 30, wherein the affinity, e.g., the combined affinity, for the MPL of the first and the second MPL-targeting moieties is at least 2, 5, 10, 20, 30, 40, 50, 75 or 100 times greater than the affinity of each targeting moiety (either alone or as part of the multispecific molecule) for its corresponding antigen binding site.

32. The multispecific molecule or MPL-binding molecule of any one of claim 16-19 or 23-29, wherein the affinity, e.g., the combined affinity, of the first and the second MPL-targeting moieties for an MPL protein expressing cell, e.g., a cancer cell or a hematopoietic cell, is equal to or greater than the affinity of a ligand, e.g., a natural ligand of an MPL protein (e.g., TPO), for the MPL protein expressing cell, e.g., a cancer cell or a hematopoietic cell.

33. The multispecific molecule or MPL-binding molecule of claim 32, wherein the affinity, e.g., the combined affinity, of the first and the second MPL-targeting moieties for the MPL protein expressing cell, e.g., a cancer cell or a hematopoietic cell, is at least 2, 5, 10, 20, 30, 40, 50, 75 or 100 times greater than the affinity of the ligand, e.g., a natural ligand of an MPL protein (e.g., TPO), for the MPL protein expressing cell, e.g., a cancer cell or a hematopoietic cell.

34. The multispecific molecule or MPL-binding molecule of any one of claims 14-33, wherein the MPL-targeting moiety is a full-length antibody, or an antigen-binding fragment (e.g., a Fab, F(ab′)2, Fv, a single chain Fv, a single domain antibody, a half-arm antibody, a diabody (dAb), a bivalent antibody, a monovalent antibody, or a bispecific antibody or fragment thereof, a single domain variant thereof, or a camelid antibody).

35. The multispecific molecule of any one of claim 14-19 or 23-34, wherein the immunoglobulin constant region (e.g., an Fc region) is linked, e.g., covalently linked to two MPL-targeting moieties, e.g., MPL-targeting moieties with non-overlapping antigen binding sites.

36. The multispecific molecule or MPL-binding molecule of any one of claims 14-35, wherein the MPL-targeting moiety comprises a light chain constant region chosen from the light chain constant region of kappa or lambda, or a fragment thereof.

37. The multispecific molecule of any one of claim 14-19 or 23-36, which comprises a first MPL-targeting moiety and a second MPL-targeting moiety, wherein the first MPL-targeting moiety comprises a kappa light chain constant region, or a fragment thereof, and the second MPL-targeting moiety comprises a lambda light chain constant region, or a fragment thereof.

38. The multispecific molecule of any one of claim 14-19 or 23-37, which comprises a first MPL-targeting moiety and a second MPL-targeting moiety, wherein the first MPL-targeting moiety and the second MPL-targeting moiety comprise a common light chain variable region.

39. The multispecific molecule or MPL-binding molecule of any one of claims 14-38, which comprises a dimerization domain, e.g., an interface of a first and second immunoglobulin chain constant regions (e.g., Fc region).

40. The multispecific molecule or MPL-binding molecule of claim 39, wherein the dimerization domain is engineered, e.g., mutated, to increase or decrease dimerization, e.g., relative to a non-engineered interface.

41. The multispecific molecule or MPL-binding molecule of claim 40, wherein the dimerization of the immunoglobulin chain constant regions (e.g., Fc regions) is enhanced by providing an Fc interface of a first and a second Fc region with one or more of: a paired cavity-protuberance (“knob-in-a hole”), an electrostatic interaction, or a strand-exchange, such that a greater ratio of heteromultimer:homomultimer forms, e.g., relative to a non-engineered interface.

42. The multispecific molecule or MPL-binding molecule of claim 39 or 40, wherein the immunoglobulin chain constant region (e.g., Fc region) comprises an amino acid substitution at a position chosen from one or more of 347, 349, 350, 351, 366, 368, 370, 392, 394, 395, 397, 398, 399, 405, 407, or 409, e.g., of the Fc region of human IgG1.

43. The multispecific molecule or MPL-binding molecule of any one of claims 39-42, wherein the immunoglobulin chain constant region (e.g., Fc region) comprises an amino acid substitution chosen from: T366S, L368A, or Y407V (e.g., corresponding to a cavity or hole), or T366W (e.g., corresponding to a protuberance or knob), or a combination thereof.

44. A multispecific molecule comprising a first antigen-binding domain and a second antigen-binding domain, wherein:
i) the first and the second antigen-binding domains bind different epitopes on a single MPL protein (e.g., bind non overlapping epitopes); or
ii) the first antigen-binding domain binds to MPL and the second antigen-binding domain binds to an antigen other than MPL, e.g., a tumor antigen other than MPL, and wherein:
the first antigen-binding domain comprises a first polypeptide and a second polypeptide, and the second antigen-binding domain comprises a third polypeptide and a fourth polypeptide, wherein:
a) the first polypeptide comprises, e.g., in the N- to C-orientation, a first heavy chain variable region (VH), a first heavy chain constant region 1 (CH1), and optionally a first region that promotes association of the first and third polypeptides, e.g., a first Fc region (e.g., a first CH2-CH3);
b) the second polypeptide comprises, e.g., in the N- to C-orientation, a first light chain variable region (VL) and a first light chain constant region (CL);
c) the third polypeptide comprises, e.g., in the N- to C-orientation, a second heavy chain variable region (VH), a second heavy chain constant region 1 (CH1), and optionally, a second region that promotes association of the first and third polypeptides, e.g., a second Fc region (e.g., a second CH2-CH3); and
d) the fourth polypeptide comprises, e.g., in the N- to C-orientation, a second light chain variable region (VL) and a second light chain constant region (CL).

45. A multispecific molecule comprising a first antigen-binding domain and a second antigen-binding domain, wherein:
i) the first and the second antigen-binding domains bind different epitopes on a single MPL protein (e.g., bind non overlapping epitopes); or
ii) the first antigen-binding domain binds to MPL and the second antigen-binding domain binds to an antigen other than MPL, e.g., a tumor antigen other than MPL, and wherein:
the first antigen-binding domain comprises a first polypeptide, and the second antigen-binding domain comprises a second polypeptide, wherein:
a) the first polypeptide comprises, e.g., in the N- to C-orientation, a first scFv region comprising a first heavy chain variable region (VH) and a first light chain variable region (VL), and optionally, a first region that promotes association of the first and second polypeptides, e.g., a first Fc region (e.g., a first CH2-CH3);
b) the second polypeptide comprises, e.g., in the N- to C-orientation, a second scFv region comprising a second VH and a second VL, and optionally, a second region that promotes association of the first and second polypeptides, e.g., a second Fc region (e.g., a second CH2-CH3).

46. An MPL-binding molecule comprising:
i) a single MPL-targeting moiety comprising a first polypeptide and a second polypeptide; and
ii) a third polypeptide, wherein:
a) the first polypeptide comprises, e.g., in the N- to C-orientation, a heavy chain variable region (VH) and a heavy chain constant region 1 (CH1), and optionally, a first region that promotes association of the first and third polypeptides, e.g., a first Fc region (e.g., a first CH2-CH3);
b) the second polypeptide comprises, e.g., in the N- to C-orientation, a light chain variable region (VL) and a light chain constant region (CL); and
c) the third polypeptide comprises, e.g., in the N- to C-orientation, a second region that promotes association of the first and third polypeptides, e.g., a second Fc region (e.g., a second CH2-CH3).

47. An MPL-binding molecule comprising a single MPL-targeting moiety comprising an scFv comprising a heavy chain variable region (VH) and a light chain variable region (VL), wherein:
i) the MPL-binding molecule further comprises an immunoglobulin constant domain, e.g., an Fc constant region, e.g., a CH2-CH3; and/or
ii) the MPL-binding molecule reduces, e.g., inhibits, an MPL activity.

48. An MPL-binding molecule comprising one or two MPL-targeting moieties, wherein the MPL-binding molecule reduces an MPL activity, and wherein the one or two MPL-targeting moieties comprise:
(i) one, two, or three CDRs from any of the heavy chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the heavy chain variable domain sequences of Table 1; or
(ii) a heavy chain variable domain sequence chosen from any of the heavy chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

49. The MPL-binding molecule of claim 48, wherein the one or two MPL-targeting moieties further comprise:
(i) one, two, or three CDRs from any of the light chain variable domain sequences of Table 1, or a closely related CDR, e.g., a CDR which has at least one amino acid alteration, but not more than two, three or four alterations (e.g., substitutions, deletions, or insertions, e.g., conservative substitutions) from any of the CDR sequences of any of the light chain variable domain sequences of Table 1; or
(ii) a light chain variable domain sequence chosen from any of the light chain variable domain amino acid sequences of Table 1, or an amino acid sequence substantially identical thereto (e.g., 95% to 99.9% identical thereto), or having at least one amino acid alteration, but not more than five, ten or fifteen alterations (e.g., substitutions, deletions, or insertions, e.g., conservative substitutions).

50. The multispecific molecule or MPL-binding molecule of any one of claims 14-49, which binds preferentially to an MPL associated with a mutated JAK2 (e.g., a JAK2 comprising a V617F mutation) over an MPL associated with a wild-type JAK2.

51. The multispecific molecule or MPL-binding molecule of claim 50, wherein:
i) the multispecific molecule or MPL-binding molecule binds to an MPL associated with a mutated JAK2 (e.g., a JAK2 comprising a V617F mutation) with a greater affinity, e.g., at least 2, 5, 10, 20, 30, 40, 50, 75 or 100 times greater affinity, than when the multispecific molecule or MPL-binding molecule binds to an MPL associated with a wild-type JAK2;
ii) the multispecific molecule or MPL-binding molecule binds to an epitope that is only present in MPL when MPL is associated with a mutated JAK2 (e.g., a JAK2 comprising a V617F mutation), but not when MPL is associated with a wild-type JAK2.

52. An MPL-binding molecule that binds preferentially to an MPL associated with a mutated JAK2 (e.g., a JAK2 comprising a V617F mutation) over an MPL associated with a wild-type JAK2.

53. The MPL-binding molecule of claim 52, wherein:
i) the MPL-binding molecule binds to an MPL associated with a mutated JAK2 (e.g., a JAK2 comprising a V617F mutation) with a greater affinity, e.g., at least 2, 5, 10, 20, 30, 40, 50, 75 or 100 times greater affinity, than when the MPL-binding molecule binds to an MPL associated with a wild-type JAK2;
ii) the MPL-binding molecule binds to an epitope that is only present in MPL when MPL is associated with a mutated JAK2 (e.g., a JAK2 comprising a V617F mutation), but not when MPL is associated with a wild-type JAK2.

54. The multispecific molecule or MPL-binding molecule of any one of claims 14-53, further comprising a targeting moiety that binds to a phosphatase, e.g., a protein tyrosine phosphatase (PTP), e.g., a receptor protein tyrosine phosphatase (RPTP).

55. The multispecific molecule or MPL-binding molecule of claim 54, wherein the phosphatase and MPL are expressed in a same cell, e.g., a myelofibrosis cell.

56. The multispecific molecule or MPL-binding molecule of claim 54 or 55, wherein the phosphatase can dephosphorylate MPL or a molecule that interacts directly or indirectly with MPL (e.g., a tyrosine kinase that interacts directly or indirectly with MPL, e.g., JAK2 or Src).

57. The multispecific molecule or MPL-binding molecule of any one of claims 54-56, wherein the phosphatase is selected from the group consisting of CD45, RPTPμ, RPTPκ, RPTPρ, RPTPλ, leukocyte antigen-related tyrosine phosphatase (LAR), RPTPσ, RPTPδ, RPTPβ, CD148, SAP1, RPTPO, RPTPQ/PTPS31, RPTPα, RPTPε, RPTPζ, RPTPγ, PC-PTP, IA2, and IA2β.

58. The multispecific molecule or MPL-binding molecule of any one of claims 54-56, wherein the phosphatase is CD45, optionally wherein the targeting moiety that binds to a phosphatase binds to one or more of CD45RA, CD45RB, CD45RC, CD45RAB, CD45RAC, CD45RBC, CD45RO, or CD45R (ABC).

59. The multispecific molecule or MPL-binding molecule of any one of claims 54-56, wherein the phosphatase is CD148.

60. The multispecific molecule or MPL-binding molecule of any one of claims 54-56, wherein the phosphatase is LAR.

61. An isolated nucleic acid molecule encoding the multispecific molecule or the MPL-binding molecule of any one of claims 1-60.

62. A vector, e.g., an expression vector, comprising the isolated nucleic acid molecule of claim 61.

63. A host cell comprising the nucleic acid molecule of claim 61 or the vector of claim 62.

64. A method of making, e.g., producing, the multispecific molecule or the MPL-binding molecule of any one of claims 1-60, comprising culturing the host cell of claim 63, under suitable conditions, e.g., conditions suitable for gene expression and/or homo- or heterodimerization.

65. A pharmaceutical composition comprising the multispecific molecule or the MPL-binding molecule of any one of claims 1-60 and a pharmaceutically acceptable carrier, excipient, or stabilizer.

66. A method of treating a cancer, comprising administering to a subject in need thereof the multispecific molecule or the MPL-binding molecule of any one of claims 1-60, wherein the multispecific antibody is administered in an amount effective to treat the cancer.

67. The method of claim 66, wherein the cancer is chosen from a hematological cancer, a B-cell or T cell malignancy, e.g., Hodgkin's lymphoma, Non-Hodgkin's lymphoma (e.g., B cell lymphoma, diffuse large B cell lymphoma, follicular lymphoma, chronic lymphocytic leukemia, mantle cell lymphoma, marginal zone B-cell lymphoma, Burkitt lymphoma, lymphoplasmacytic lymphoma, hairy cell leukemia), myelofibrosis, acute myeloid leukemia (AML), chronic myeloid leukemia (CML), myelodysplastic syndrome, multiple myeloma, or acute lymphocytic leukemia (ALL).

68. The method of claim 66, wherein the cancer is myelofibrosis.
